SEQUENCE LISTING (92 sequences)

SEQ ID NO: 1 - protein, 157 aa, Arabidopsis thaliana
MSDSASSNTKAIDPPLHMLGFEFDELSPTRITGRLPVSPVCCQPFKVLHGGVSALIAESLASMGAHMASGFKRVAGIQLSINHLKSADLGDLVFAEATPVSTGKTIQVWEVKLWKTTQKDKANKILISSSRVTLICNLPIPDNAKDAANMLKMVAKL

SEQ ID NO: 2 - protein, 165 aa, Petunia hybrida
MSKPPPLSSSAAGIKIREIDIPLHEIGFEYVEITPHKISGRLHLTERCCQPFKVLHGGISALIAESLASMGAHIASGFTRVAGVHLSIHHLKSAQLGDLVIAEAAPVNVGQSIQVWEVCLWKIYESNEEEKKTLIASSRVTLKTNMFVPENAKDAAVVLKKYAKL

SEQ ID NO: 3 - protein, 502 aa, Arabidopsis thaliana
MSENGKCNGATTVKLPEIKFTKLFINGQFIDAASGKTFETIDPRNGEVIATIAEGDKEDVDLAVNAARYAFDHGPWPRMTGFERAKLINKFADLIEENIEELAKLDAVDGGKLFQLGKYADIPATAGHFRYNAGAADKIHGETLKMTRQSLFGYTLKEPIGVVGNIIPWNFPSIMFATKVAPAMAAGCTMVVKPAEQTSLSALFYAHLSKEAGIPDGVLNIVTGFGSTAGAAIASHMDVDKVSFTGSTDVGRKIMQAAAASNLKKVSLELGGKSPLLIFNDADIDKAADLALLGCFYNKGEICVASSRVFVQEGIYDKVVEKLVEKAKDWTVGDPFDSTARQGPQVDKRQFEKILSYIEHGKNEGATLLTGGKAIGDKGYFIQPTIFADVTEDMKIYQDEIFGPVMSLMKFKTVEEGIKCANNTKYGLAAGILSQDIDLINTVSRSIKAGIIWVNCYFGFDLDCPYGGYKMSGNCRESGMDALDNYLQTKSVVMPLHNSPWM

SEQ ID NO: 4 - protein, 342 aa, Populus tomentosa
MSPVDISSLPCQGQTVCVTGAGGFIASWIVKLLLEKGYSVKGTVRNPADPKNSHLRELEGAQERLTLCKADILDYESLKMAIQGCDGVFHTACPITDDPDMVMAPAVNGTKNVIMAAAETKVRRVVVTSSIGTVYMDPNRSPDVVVDESCWSDLEFCKSTKNWYCYGRTVAEQVAWDVAKKKGVDLVVVNPVMVLGPLLQPTVNASIGHILKYLTGSAKTYANSVQAYVHVRDVALAHILVFETPSASGRYICAERMLHRGEVVEILAKFFPEYPIPTKCSDEKNPRKQPYKFTNQKIKDLGIEFTPVKQCLYETVKSLQEKGHLPIPKQAQDSSVVGISKY

SEQ ID NO: 5 - protein, 562 aa, Arabidopsis thaliana
MSAPQEQAVSQVMEKQSNNNNSDVIFRSKLPDIYIPNHLSLHDYIFQNISEFATKPCLINGPTGHVYTYSDVHVISRQIAANFHKLGVNQNDVVMLLLPNCPEFVLSFLAASFRGATATAANPFFTPAEIAKQAKASNTKLIITEARYVDKIKPLQNDDGVVIVCIDDNESVPIPEGCLRFTELTQSTTEASEVIDSVEISPDDVVALPYSSGTTGLPKGVMLTHKGLVTSVAQQVDGENPNLYFHSDDVILCVLPMFHIYALNSIMLCGLRVGAAILIMPKFEINLLLELIQRCKVTVAPMVPPIVLAIAKSSETEKYDLSSIRVVKSGAAPLGKELEDAVNAKFPNAKLGQGYGMTEAGPVLAMSLGFAKEPFPVKSGACGTVVRNAEMKIVDPDTGDSLSRNQPGEICIRGHQIMKGYLNNPAATAETIDKDGWLHTGDIGLIDDDDELFIVDRLKELIKYKGFQVAPAELEALLIGHPDITDVAVVAMKEEAAGEVPVAFVVKSKDSELSEDDVKQFVSKQVVFYKRINKVFFTESIPKAPSGKILRKDLRAKLANGL

SEQ ID NO: 6 - protein, 560 aa, Citrus clementina
MISIATKKPELSLDISSPAPPAPSNEKIATHIFKSKLPDIPISNHLPLHTYCFQDRLSDDPCLIVGLTGKTYSYAETHLICRKTAAGLSNLGIKKGDVIMILLQNCAEFVFSFMGASMIGAVTTTANPFYTSAEILKQFRTSGAKLIITMSQYVDRLPKTDKDFTVITIDAPPENCLHFTVLSEADEDQIPEVAIEPDDPVALPFSSGTTGLPKGVVLTHKSLITSVAQQVDGENPNLYLTNGDVVLCVLPLFHIYSLNSVLLCSLRAGAGVLLMQKFEIGALLELIQRHRVSVAAVVPPLVLALAKNPMVADYDLSSIRVVLSGAAPLGKELEDALRSRVPQAILGQGYGMTEAGPVLSMCLGFAKQPFPTKSGSCGTVVRNAELKVIDPEIGASLPHNQPGEICIRGPQIMKGYLNDPEATAATIDVEGWLHTGDIGYVDDDDEVFIVDRVKEIIKFKGFQVPPAEIEALLLSHPSIGDAAVVPQKDEVAGEVPVAFVVRSNGFELTEEAIKEYIAKQVVFYKRLHKIYFVHAIPKSPSGKILRKDLRAKLASSMPLN

SEQ ID NO: 7 - protein, 571 aa, Arabidopsis thaliana
MSVLQQQTHFLTKKIDQEDEEEEPSHDFIFRSKLPDIFIPNHLPLTDYVFQRFSGDGDGDSSTTCIIDGATGRILTYADVQINMRRIATGIHRLGIRHGDVVMLLLPNSPEFALSFLAVAYLGAVSTTANPFYTQPEIAKQAKASAAKMIITKKCLVDKLTNLKNDGVLIVCLDDDGDNGVVSSSDDGCVSFTELTQADETELLKPKISPEDTVAMPYSSGTTGLPKGVMITHKGLVTSIAQKVDGENPNLNFTANDVILCFLPMFHIYALDALMLSAMRTGAALLIVPRFELNLVMELIQRYKVTVVPVAPPVVLAFIKSPETERYDLSSVRIMLSGAATLKKELEDAVRLKFPNAIFGQGYGMTESGTVAKSLAFAKNPFKTKSGACGTVIRNAEMKVVDTETGISLPRNKSGEICVRGHQLMKGYLNDPEATARTIDKDGWLHTGDIGFVDDDDEIFIVDRLKELIKFKGYQVAPAELEALLISHPSIDDAAVVAMKDEVADEVPVAFVARSQGSQLTEDDVKSYVNKQVVHYKRIKMVFFIEVIPKAVSGKILRKDLRAKLETMCSK

SEQ ID NO: 8 - protein, 537 aa, Populus tomentosa
MSNPQEEFIFRSKLPDIYIPKNLPLHSYVLENLSNHSSKPCLINGANGDVYTYADVELTARRVASGLNKIGIQQGDVIMLFLPSSPEFVLAFLGASHRGAIITAANPFSTPAELAKHAKASRAKLLITQACYYEKVKDFARESDVKVMCVDSAPDGCLHFSELTQADENEAPQVDISPDDVVALPYSSGTTGLPKGVMLTHKGLITSVAQQVDGDNPNLYFHSEDVILCVLPMFHIYALNSIMLCGLRVGAPILIMPKFEIGSLLGLIEKYKVSIAPVVPPVMMSIAKSPDLDKHDLSSLRMIKSGGAPLGKELEDTVRAKFPQARLGQGYGMTEAGPVLAMCLAFAKEPFDIKPGACGTVVRNAEMKIVDPETGASLPRNQPGEICIRGDQIMKGYLNDPEATSRTIDKEGWLHTGDIGYIDDDDELFIVDRLKELIKYKGFQVAPAELEALLIAHPEISDAAVVGLKDEDAGEVPVAFVVKSEKSQATEDEIKQYISKQVIFYKRIKRVFFIEAIPKAPSGKILRKNLKEKLAGI

SEQ ID NO: 9 - protein, 571 aa, Arabidopsis thaliana
MSVLQQQTHFLTKKIDQEDEEEEPSHDFIFRSKLPDIFIPNHLPLTDYVFQRFSGDGDGDSSTTCIIDGATGRILTYADVQTNMRRIAAGIHRLGIRHGDVVMLLLPNSPEFALSFLAVAYLGAVSTTANPFYTQPEIAKQAKASAAKMIITKKCLVDKLTNLKNDGVLIVCLDDDGDNGVVSSSDDGCVSFTELTQADETELLKPKISPEDTVAMPYSSGTTGLPKGVMITHKGLVTSIAQKVDGENPNLNFTANDVILCFLPMFHIYALDALMLSAMRTGAALLIVPRFELNLVMELIQRYKVTVVPVAPPVVLAFIKSPETERYDLSSVRIMLSGAATLKKELEDAVRLKFPNAIFGQGYGMTESGTVAKSLAFAKNPFKTKSGACGTVIRNAEMKVVDTETGISLPRNKSGEICVRGHQLMKGYLNDPEATARTIDKDGWLHTGDIGFVDDDDEIFIVDRLKELIKFKGYQVAPAELEALLISHPSIDDAAVVAMKDEVADEVPVAFVARSQGSQLTEDDVKSYVNKQVVHYKRIKMVFFIEVIPKAVSGKILRKDLRAKLETMCSK

SEQ ID NO: 10 - protein, 243 aa, Vitis vinifera
MSATNQEAGRHQEVGHKSLLQSDALYQYILETSVYPREPESMKELRELTAQHPWNIMTTSADEGQFLNMLLKLINAKNTMEIGVYTGYSLLATALALPDDGKILAMDINKENYELGLPVIQKAGVAHKIDFKEGPALPVLDQMIEDGKYHGSFDFIFVDADKDNYLNYHKRLIDLVKVGGIIGYDNTLWNGSVVAPPDAPLRKYVRYYRDFVLELNKALAADPRIEICMLPVGDGITLCRRLS

SEQ ID NO: 11 - protein, 248 aa, Medicago sativa
MSATNEDQKQTESGRHQEVGHKSLLQSDALYQYILETSVFPREHEAMKELREVTAKHPWNIMTTSADEGQFLSMLLKLINAKNTMEIGVYTGYSLLATALAIPEDGKILAMDINKENYELGLPVIKKAGVDHKIDFREGPALPVLDEMIKDEKNHGSYDFIFVDADKDNYLNYHKRLIDLVKVGGVIGYDNTLWNGSVVAPPDAPLRKYVRYYRDFVLELNKALAVDPRIEICMLPVGDGITICRRIK

SEQ ID NO: 12 - protein, 248 aa, Eucalyptus globus
MSAANAEPQQTQPAKHSEVGHKSLLQSDALYQYILETSVYPREPESMKELREITAKHPWNLMTTSADEGQFLNMLLKLINAKNTMEIGVYTGYSLLATALALPDDGKILAMDINRENFEIGLPVIEKAGLAHKIDFREGPALPLLDQLVQDEKNHGTYDFIFVDADKDNYINYHKRLIDLVKVGGLIGYDNTLWNGSVVAPADAPLRKYVRYYRDFVLELNKALAVDPRVEICMLPVGDGITLCRRVS

SEQ ID NO: 13 - protein, 243 aa, Nicotiana tabacum
MSATNGENGRHQEVGHKSLLQSDALYQYILETSVYPREPEPMKELREITAKHPWNLMTTSADEGQFLSMLLKLINAKNTMEIGVFTGYSLLATAMALPDDGKILAMDINRENYEIGLPIIEKAGLAHKIVFREGPALPVLDQMIEDGKYHGSYDFIFVDADKDNYLNYHKRLIDLVKVGGLIGYDNTLWNGSVVAPPDAPLRKYVRYYRDFVLELNKALAADSRIEICQLPVGDGITLCRRIS

SEQ ID NO: 14 - protein, 243 aa, Nicotiana tabacum
MSATNGENGRHQEVGHKSLLQSDALYQYILETSVYPREPEPMKELREITAKHPWNIMTTSADEGQFLSMLLKLINAKNTMEIGVFTGYSLLATAMALPDDGKILAMDINRDNYEIGLPVIEKAGLAHKIEFKEGPALPVLDQMIEDGKYHGSYDFIFVDADKDNYLNYHKRLIDLVKVGGLIGYDNTLWNGSVVAPPDAPLRKYVRYYRDFVLELNKALAADSRIEICQLPVGDGITLCRRIS

SEQ ID NO: 15 - protein, 259 aa, Arabidopsis thaliana
MSATTTTEATKTSSTNGEDQKQSQNLRHQEVGHKSLLQSDDLYQYILETSVYPREPESMKELREVTAKHPWNIMTSADEGQFLNMLIKLVNAKNTMEIGVYTGYSLLATALALPEDGKILAMDVNRENYELGLPIIEKAGVAHKIDFREGPALPVLDEIVADEKNHGTYDFIFVDADKDNYINYHKRLIDLVKIGGVIGYDNTLWNGSVVAPPDAPMRKYVRYYRDFVLELNKALAADPRIEICMLPVGDGITICRRIS

SEQ ID NO: 16 - protein, 248 aa, Populus trichocarpa
MSATNGEEQQSQAGRHQEVGHKSLLQSDALYQYILETSVYPREPECMKELREVTAKHPWNIMTTSADEGQFLNMLLKLVNAKNTMEIGVYTGYSLLATALAIPEDGKILAMDINRENYELGLPVIQKAGVAHKIDFKEGPALPVLDQMIEDGKCHGSFDFIFVDADKDNYINYHKRLIELVKVGGLIGYDNTLWNGSVVAPPDAPMRKYVRYYRDFVLELNKALAADPRIEICMLPVGDGITLCRRIQ

SEQ ID NO: 17 - protein, 521 aa, Pseudomonas aeruginosa
MSKPEDFRASATRPFTGEEYLASLRDDREIYIYGDRVKDVTSHPAFRNAAASMARLYDALHDPQSKEKLCWETDTGNGGYTHKFFRYARSADELRQQRDAIAEWSRLTYGWMGRTPDYKAAFGSALGANPGFYGRFEDNAKTWYKRIQEACLYLNHAIVNPPIDRDKPVDQVKDVFISVDEEVDGGIVVSGAKVVATNSALTHYNFVGQGSAQLLGDNTDFALMFIAPMNTPGMKLICRPSYELVAGIAGSPFDYPLSSRFDENDAILVMDKVFIPWENVLIYRDFERCKQWFPQGGFGRLFPMQGCTRLAVKLDFITGALYKALQCTGSLEFRGVQAQVGEVVAWRNLFWSLTDAMYGNASEWHGGAFLPSAEALQAYRVLAPQAYPEIKKTIEQVVASGLIYLPSGVRDLHNPQLDKYLSTYCRGSGGMGHRERIKILKLLWDAIGSEFGGRHELYEINYAGSQDEIRMQALRQAIGSGAMKGMLGMVEQCMGDYDENGWTVPHLHNPDDINVLDRIRQ

SEQ ID NO: 18 - protein, 171 aa, Salmonella enterica
MSQVDEQRLRFRDAMASLAAAVNIVTTAGHAGRCGITATAVCSVTDTPPSVMVCINANSAMNPVFQGNGRLCINVLNHEQELMARHFAGMTGMAMEERFHQPCWQNGPLGQPVLNGALAGLEGEISEVQTIGTHLVYLVAIKNIILSQDGHGLIYFKRRFHPVRLEMEAPV

SEQ ID NO: 19 - protein, 693 aa, Rhodotorula glutinis
SAPRPTSQSQARTCPTTQVTQVDIVEKMLAAPTDSTLELDGYSLNLGDVVSAARKGRPVRVKDSDEIRSKIDKSVEFLRSQLSMSVYGVTTGFGGSADTRTEDAISLQKALLEHQLCGVLPSSFDSFRLGRGLENSLPLEVVRGAMTIRVNSLTRGHSAVRLVVLEALTNFLNHGITPIVPLRGTISASGDLSPLSYIAAAISGHPDSKVHVVHEGKEKILYAREAMALFNLEPVVLGPKEGLGLVNGTAVSASMATLALHDAHMLSLLSQSLTAMTVEAMVGHAGSFHPFLHDVTRPHPTQIEVAGNIRKLLEGSRFAVHHEEEVKVKDDEGILRQDRYPLRTSPQWLGPLVSDLIHAHAVLTIEAGQSTTDNPLIDVENKTSHHGGNFQAAAVANTMEKTRLGLAQIGKLNFTQLTEMLNAGMNRGLPSCLAAEDPSLSYHCKGLDIAAAAYTSELGHLANPVTTHVQPAEMANQAVNSLALISARRTTESNDVLSLLLATHLYCVLQAIDLRAIEFEFKKQFGPAIVSLIDQHFGSAMTGSNLRDELVEKVNKTLAKRLEQTNSYDLVPRWHDAFSFAAGTVVEVLSSTSLSLAAVNAWKVAAAESAISLTRQVRETFWSAASTSSPALSYLSPRTQILYAFVREELGVKARRGDVFLGKQEVTIGSNVSKIYEAIKSGRINNVLLKMLA

SEQ ID NO: 20 - protein, 718 aa, Arabidopsis thaliana
MSDQIEAMLCGGGEKTKVAVTTKTLADPLNWGLAADQMKGSHLDEVKKMVEEYRRPVVNLGGETLTIGQVAAISTVGGSVKVELAETSRAGVKASSDWVMESMNKGTDSYGVTTGFGATSHRRTKNGTALQTELIRFLNAGIFGNTKETCHTLPQSATRAAMLVRVNTLLQGYSGIRFEILEAITSLLNHNISPSLPLRGTITASGDLVPLSYIAGLLTGRPNSKATGPDGESLTAKEAFEKAGISTGFFDLQPKEGLALVNGTAVGSGMASMVLFEANVQAVLAEVLSAIFAEVMSGKPEFTDHLTHRLKHHPGQIEAAAIMEHILDGSSYMKLAQKVHEMDPLQKPKQDRYALRTSPQWLGPQIEVIRQATKSIEREINSVNDNPLIDVSRNKAIHGGNFQGTPIGVSMDNTRLAIAAIGKLMFAQFSELVNDFYNNGLPSNLTASSNPSLDYGFKGAEIAMASYCSELQYLANPVTSHVQSAEQHNQDVNSLGLISSRKTSEAVDILKLMSTTFLVGICQAVDLRHLEENLRQTVKNTVSQVAKKVLTTGINGELHPSRFCEKDLLKVVDREQVFTYVDDPCSATYPLMQRLRQVIVDHALSNGETEKNAVTSIFQKIGAFEEELKAVLPKEVEAARAAYGNGTAPIPNRIKECRSYPLYRFVREELGTKLLTGEKVVSPGEEFDKVFTAMCEGKLIDPLMDCLKEWNGAPIPIC

SEQ ID NO: 21 - protein, 506 aa, Arabidopsis thaliana
MSDLLLLEKSLIAVFVAVILATVISKLRGKKLKLPPGPIPIPIFGNWLQVGDDLNHRNLVDYAKKFGDLFLLRMGQRNLVVVSSPDLTKEVLLTQGVEFGSRTRNVVFDIFTGKGQDMVFTVYGEHWRKMRRIMTVPFFTNKVVQQNREGWEFEAASVVEDVKKNPDSATKGIVLRKRLQLMMYNNMFRIMFDRRFESEDDPLFLRLKALNGERSRLAQSFEYNYGDFIPILRPFLRGYLKICQDVKDRRIALFKKYFVDERKQIASSKPTGSEGLKCAIDHILEAEQKGEINEDNVLYIVENINVAAIETTLWSIEWGIAELVNHPEIQSKLRNELDTVLGPGVQVTEPDLHKLPYLQAVVKETLRLRMAIPLLVPHMNLHDAKLAGYDIPAESKILVNAWWLANNPNSWKKPEEFRPERFFEEESHVEANGNDFRYVPFGVGRRSCPGIILALPILGITIGRMVQNFELLPPPGQSKVDTSEKGGQFSLHILNHSIIVMKPRNC

SEQ ID NO: 22 - protein, 714 aa, Catharantus roseus
SDSSSEKLSPFELMSAILKGAKLDGSNSSDSGVAVSPAVMAMLLENKELVMILTTSVAVLIGCVVVLIWRRSSGSGKKVVEPPKLIVPKSVVEPEEIDEGKKKFTIFFGTQTGTAEGFAKALAEEAKARYEKAVIKVIDIDDYAADDEEYEEKFRKETLAFFILATYGDGEPTDNAARFYKWFVEGNDRGDWLKNLQYGVFGLGNRQYEHFNKIAKVVDEKVAEQGGKRIVPLVLGDDDQCIEDDFAAWRENVWPELDNLLRDEDDTTVSTTYTAAIPEYRVVFPDKSDSLISEANGHANGYANGNTVYDAQHPCRSNVAVRKELHTPASDRSCTHLDFDIAGTGLSYGTGDHVGVYCDNLSETVEEAERLLNLPPETYFSLHADKEDGTPLAGSSLPPPFPPCTLRTALTRYADLLNTPKKSALLALAAYASDPNEADRLKYLASPAGKDEYAQSLVANQRSLLEVMAEFPSAKPPLGVFFAAIAPRLQPRFYSISSSPRMAPSRIHVTCALVYEKTPGGRIHKGVCSTWMKNAIPLEESRDCSWAPIFVRQSNFKLPADPKVPVIMIGPGTGLAPFRGFLQERLALKEEGAELGTAVFFFGCRNRKMDYIYEDELNHFLEIGALSELLVAFSREGPTKQYVQHKMAEKASDIWRMISDGAYVYVCGDAKGMARDVHRTLHTIAQEQGSMDSTQAEGFVKNLQMTGRYLRDVW

SEQ ID NO: 23 - protein, 370 aa, synthetic construct
  REGION 1..370: note = Mutant ARO4 K229L
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTLHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 24 - protein, 256 aa, synthetic construct
  REGION 1..256: note = Mutant ARO7 G141S
MDFTKPETVLNLQNIRDELVRMEDSIIFKFIERSHFATCPSVYEANHPGLEIPNFKGSFLDWALSNLEIAHSRIRRFESPDETPFFPDKIQKSFLPSINYPQILAPYAPEVNYNDKIKKVYIEKIIPLISKRDGDDKNNFSSVATRDIECLQSLSRRIHFGKFVAEAKFQSDIPLYTKLIKSKDVEGIMKNITNSAVEEKILERLTKKAEVYGVDPTNESGERRITPEYLVKIYKEIVIPITKEVEVEYLLRRLEE

SEQ ID NO: 25 - protein, 513 aa, Saccharothrix espanaensis
MSTITSPAPAGRLNNVRPMTGEEYLESLRDGREVYIYGERVDDVTTHLAFRNSVRSIARLYDVLHDPASEGVLRVPTDTGNGGFTHPFFKTARSSEDLVAAREAIVGWQRLVYGWMGRTPDYKAAFFGTLDANAEFYGPFEANARRWYRDAQERVLYFNHAIVHPPVDRDRPADRTADICVHVEEETDSGLIVSGAKVVATGSAMTNANLIAHYGLPVRDKKFGLVFTVPMNSPGLKLICRTSYELMVATQGSPFDYPLSSRLDENDSIMIFDRVLVPWENVFMYDAGAANSFATGSGFLERFTFHGCTRLAVKLDFIAGCVMKAVEVTGTTHFRGVQAQVGEVLNWRDVFWGLSDAMAKSPNSWVGGSVQPNLNYGLAYRTFMGVGYPRIKEIIQQTLGSGLIYLNSSAADWKNPDVRPYLDRYLRGSRGIQAIDRVKLLKLLWDAVGTEFAGRHELYERNYGGDHEGIRVQTLQAYQANGQAAALKGFAEQCMSEYDLDGWTRPDLINPGT

SEQ ID NO: 26 - protein, 521 aa, Escherichia coli
MSKPEDFRASTQRPFTGEEYLKSLQDGREIYIYGERVKDVTTHPAFRNAAASVAQLYDALHKPEMQDSLCWNTDTGSGGYTHKFFRVAKSADDLRQQRDAIAEWSRLSYGWMGRTPDYKAAFGCALGANPGFYGQFEQNARNWYTRIQETGLYFNHAIVNPPIDRHLPTDKVKDVYIKLEKETDAGIIVSGAKVVATNSALTHYNMIGFGSAQVMGENPDFALMFVAPMDADGVKLISRASYEMVAGATGSPYDYPLSSRFDENDAILVMDNVLIPWENVLIYRDFDRCRRWTMEGGFARMYPLQACVRLAVKLDFITALLKKSLECTGTLEFRGVQADLGEVVAWRNTFWALSDSMCSEATPWVNGAYLPDHAALQTYRVLAPMAYAKIKNIIERNVTSGLIYLPSSARDLNNPQIDQYLAKYVRGSNGMDHVQRIKILKLMWDAIGSEFGGRHELYEINYSGSQDEIRLQCLRQAQNSGNMDKMMAMVDRCLSEYDQDGWTVPHLHNNDDINMLDKLLK

SEQ ID NO: 27 - protein, 171 aa, Escherichia coli
MSQLDEQRLRFRDAMASLSAAVNIITTEGDAGQCGITATAVCSVTDTPPSLMVCINANSAMNPVFQGNGKLCVNVLNHEQELMARHFAGMTGMAMEERFSLSCWQKGPLAQPVLKGSLASLEGEIRDVQAIGTHLVYLVEIKNIILSAEGHGLIYFKRRFHPVMLEMEAAI

SEQ ID NO: 28 - protein, 413 aa, Rhodopseudomonas palustris
MSTTAPSLVPVTTPSQHGAGVPHLGIDPFALDYFADPYPEQETLREAGPVVYLDKWNVYGVARYAEVYAVLNDPLTFCSSRGVGLSDFKKEKPWRPPSLILEADPPAHTRTRAVLSKVLSPATMKRLRDGFAAAADAKIDELLARGGNIDAIADLAEAYPLSVFPDAMGLKQEGRENLLPYAGLVLNAFGPPNELRQSAIERSAPHQAYVAEQCQRPNLAPGGFGACIHAFSDTGEITPEEAPLLVRSLLSAGLDTTVNGIAAAVYCLARFPDEFARLRADPSLARNAFEEAVRFESPVQTFFRTTTRDVELAGATIGEGEKVLMFLGSANRDPRRWDDPDRYDITRKTSGHVGFGSGVHMCVGQLVARLEGEVVLAALARKVAAIEIAGPLKRRFNNTLRGLESLPIQLTPA

SEQ ID NO: 29 - protein, 498 aa, Beta vulgaris
MSDHATLAMILAILFISFHFIKLLFSQQTTKLLPPGPKPLPIIGNILEVGKKPHRSFANLAKIHGPLISLRLGSVTTIVVSSADVAKEMFLKKDHPLSNRTIPNSVTAGDHHKLTMSWLPVSPKWRNFRKITAVHLLSPQRLDACQTFRHAKVQQLYEYVQECAQKGQAVDIGKAAFTTSLNLLSKLFFSVELAHHKSHTSQEFKELIWNIMEDIGKPNYADYFPILGCVDPSGIRRRLACSFDKLIAVFQGIICERLAPDSSTTTTTTTDDVLDVLLQLFKQNELTMGEINHLLVDIFDAGTDTTSSTLEWVMTELIRNPEMMEKAQEEIKQVLGKDKQIQESDIINLPYLQAIIKETLRLHPPTVFLLPRKADTDVELYGYIVPKDAQILVNLWAIGRDPNAWQNADIFSPERFIGCEIDVKGRDFGLLPFGAGRRICPGMNLAIRMLTLMLATLLQFFNWKLEGDISPKDLDMDEKFGIALQKTKPLKLIPIPRY

SEQ ID NO: 30 - protein, 507 aa, Flavobacterium johnsoniae
MSNTINEYLSLEEFEAIIFGNQKVTISDVVVNRVNESFNFLKEFSGNKVIYGVNTGFGPMAQYRIKESDQIQLQYNLIRSHSSGTGKPLSPVCAKAAILARLNTLSLGNSGVHPSVINLMSELINKDITPLIFEHGGVGASGDLVQLSHLALVLIGEGEVFYKGERRPTPEVFEIEGLKPIQVEIREGLALINGTSVMTGIGVVNVYHAKKLLDWSLKSSCAINELVQAYDDHFSAELNQTKRHKGQQEIALKMRQNLSDSTLIRKREDHLYSGENTEEIFKEKVQEYYSLRCVPQILGPVLETINNVASILEDEFNSANDNPIIDVKNQHVYHGGNFHGDYISLEMDKLKIVITKLTMLAERQLNYLLNSKINELLPPFVNLGTLGFNFGMQGVQFTATSTTAESQMLSNPMYVHSIPNNNDNQDIVSMGTNSAVITSKVIENAFEVLAIEMITIVQAIDYLGQKDKISSVSKKWYDEIRNIIPTFKEDQVMYPFVQKVKDHLINN

SEQ ID NO: 31 - protein, 719 aa, Citrus sinensis
MSEIGATTENGHQNGGLEGLCKNNNYNYSSGDALNWGVMAETLKGSHLEEVKRMVAEYRKPVVNLGGETLTVAQVAAIATSSTNVELSESAREGVKASSDWVMESMNKGTDSYGVTTGFGATSHRRTKNGGALQKELIRFLNAGIFGNGTESSHTLPHSATRAAMLVRVNTLLQGYSGIRFEILEAITKLLNHNITPCLPLRGTITASGDLVPLSYIAGLLTGRPNSKATGPNGEIIDAQEASKQAGFGFFELQPKEGLALVNGTAVGSGLASMVLFEANNLALLSEILSAIFAEVMQGKPEFTDHLTHKLKHHPGQIEAAAIMEHILDGSSYVNVAKKLHEIDPLQKPKQDRYALRTSPQWLGPQIEVIRFATKSIEREINSVNDNPLIDVSRNKALHGGNFQGTPIGVSMDNTRLAIAAIGKLMFAQFSELVNDFYNNGLPSNLSGGRNPSLDYGFKGAEIAMASYCSELQFLANPVTNHVQSAEQHNQDVNSLGLISSRKTAEAVDILKLMSSTFLVALCQAIDLRHLEENLKHTVKNTVSQVAKKVLTVGASGELHPSRFCEKDLLKAADREHVFAYIDDPCSATYPLMQKLRQVLVEHALNNGENEKTANSSIFQKIAAFEEELKTVLPKEVENARQTVENGSPTIPNRIKECRSYPLYRFVREGLGSNFLTGEKVTSPGEEFDKVFTAMCQGKIIDPMLECLREWNGAPLPIC

SEQ ID NO: 32 - protein, 715 aa, Citrus sinensis
MSEASHENQSGGNIPSGKLCTNIDPLNWVSASESLKGSHLDEVKRMVSEYRKQVVRLGGETLTIAQVAAVASRDGGVTVELNEEARAGVKASSDWVMESMNKGTDSYGITTGFGATSHRRTKQGAALQKELIRFLNAGIFGKGTESCQMLPHTATRAAMLVRINTLLQGYSGIRFEILEAITKFLNRNITPCLPLRASITASGDLIPFSYIAGLLTGRLNSVAVGPNGESLNAAEAFSQAGIDGGFFELQPKEGLALVNGTGVGAGLASIVLFEANILTVLSEVLSAIFAEAMLGKPEFTDHLTHKLKHHPGQIEAAAIMEHILDGSSYVKAAQKLHEIDPLQKPKQDRYALRTSPQWLGPQAEVIRASTKSIEREINSVNDNPLIDVSRNKALHGGNFQGTPIGVSMDNSRLAIASIGKLMFAQFSELVNDFYSNGLPSNLSGGRNPSLDYGFKGAEIAMAAYCSELQFLANPVTNHVQSAEQHNQDVNSLGLISARKTAEAVDILKLMSSTYLIALCQAIDLRHLEENLKSTVKNTISQVVKKVLTMGVNGELHPSRFCEKDLLKVVDREYVFSYADDPCSATYPLMQKLRQVLVDHALTNNEDLKNANASIFLKIGAFEEELKTLLPKEVESARSAFESGNLEIPNRIKECRSYPLYRFVREELGARYLTGEKAISPGEECDKVFTAICQGKIIDPLLECLKEWDGSPLPIC

SEQ ID NO: 33 - protein, 537 aa, Citrus sinensis
MSANLVTISFFSILLTISLLSFNKSLNLISITLPLVPLIAYVLKSFLKSSKAFYPPTPISIPIFGNWLQVGNDLNHRLLASMAQIYGPVFRLKLGSKNLIVVSEPDLATQVLHTQGVEFGSRPRNVVFDIFTGNGQDMVFTVYGEHWRKMRRIMTLPFFTNKVVHNYSDMWEQEMDLVVHDLKNDYESVSTKGIVIRKRLQLMLYNIMYRMMFDAKFESQEDPLFIEATRFNSERSRLAQSFEYNYGDFIPLLRPFLRGYLNKCRDLQCRRLAFFNNNFVEKRRKIMAANGEKHKISCAIDHIIDAQMKGEITEENVIYIVENINVAAIETTLWSMEWAIAELVNHPEVQQKIRREISTVLKGNPVTESNLHELPYLQAAVKEVLRLHTPIPLLVPHMNLEEAKLGGFTIPKESKIVVNAWWLANNPKWWEKPEEFRPERFLEEECNIDAVAGGGKVDFRYLPFGVGRRSCPGIILALPILGLVIAKLVTSFEMKAPQGIDKIDVSEKGGQFSLHIANHSTVVFDPIMESLSQPMPQ

SEQ ID NO: 34 - protein, 520 aa, Citrus sinensis
MSDLNGWCNSGNQNMCCCQSYVKRGYDRVLSFNGLITVSKLRGKRFKLPPGPLPVPVFGNWLQVGDDLNHRNLSDLAKKYGDVLLLRMGQRNLVVVSSPDHAKEVLHTQGVEFGSRTRNVVFDIFTGKGQDMVFTVYGEHWRKMRRIMTVPFFTNKVVQQQRFNWEDEAARVVEDVKKDPEAATNGIVLRRRLQLMMYNNMYRIMFDRRFESQDDPLFNRLKALNGERSRLAQSFEYNYGDFIPILRPFLRGYLKICKEVKERRLQLFKDYFVEERKKLASTKSMSNESLKCAIDHILDAQTKGEINEDNVLYIVENINVAAIETTLWSIEWGIAELVNHPEIQKKLRNELDTVLGPGHQITEPDTHKLPYLQAVIKETLRLRMAIPLLVPHMNLHDAKLGGYDVPAESKILVNAWWLANNPAQWKKPEEFRPERFLEEESKVEANGNDFRYLPFGVGRRSCPGIILALPILGITIGRLVQNFELLPPPGQSKIDTAEKGGQFSLHILKHSTIVAKPRSF

SEQ ID NO: 35 - protein, 692 aa, Saccharomyces cerevisiae
MSPFGIDNTDFTVLAGLVLAVLLYVKRNSIKELLMSDDGDITAVSSGNRDIAQVVTENNKNYLVLYASQTGTAEDYAKKFSKELVAKFNLNVMCADVENYDFESLNDVPVIVSIFISTYGEGDFPDGAVNFEDFICNAEAGALSNLRYNMFGLGNSTYEFFNGAAKKAEKHLSAAGAIRLGKLGEADDGAGTTDEDYMAWKDSILEVLKDELHLDEQEAKFTSQFQYTVLNEITDSMSLGEPSAHYLPSHQLNRNADGIQLGPFDLSQPYIAPIVKSRELFSSNDRNCIHSEFDLSGSNIKYSTGDHLAVWPSNPLEKVEQFLSIFNLDPETIFDLKPLDPTVKVPFPTPTTIGAAIKHYLEITGPVSRQLFSSLIQFAPNADVKEKLTLLSKDKDQFAVEITSKYFNIADALKYLSDGAKWDTVPMQFLVESVPQMTPRYYSISSSSLSEKQTVHVTSIVENFPNPELPDAPPVVGVTTNLLRNIQLAQNNVNIAETNLPVHYDLNGPRKLFANYKLPVHVRRSNFRLPSNPSTPVIMIGPGTGVAPFRGFIRERVAFLESQKKGGNNVSLGKHILFYGSRNTDDFLYQDEWPEYAKKLDGSFEMVVAHSRLPNTKKVYVQDKLKDYEDQVFEMINNGAFIYVCGDAKGMAKGVSTALVGILSRGKSITTDEATELIKMLKTSGRYQEDVW

SEQ ID NO: 36 - protein, 693 aa, Arabidopsis thaliana
MSTSALYASDLFKQLKSIMGTDSLSDDVVLVIATTSLALVAGFVVLLWKKTTADRSGELKPLMIPKSLMAKDEDDDLDLGSGKTRVSIFFGTQTGTAEGFAKALSEEIKARYEKAAVKVIDLDDYAADDDQYEEKLKKETLAFFCVATYGDGEPTDNAARFYKWFTEENERDIKLQQLAYGVFALGNRQYEHFNKIGIVLDEELCKKGAKRLIEVGLGDDDQSIEDDFNAWKESLWSELDKLLKDEDDKSVATPYTAVIPEYRVVTHDPRFTTQKSMESNVANGNTTIDIHHPCRVDVAVQKELHTHESDRSCIHLEFDISRTGITYETGDHVGVYAENHVEIVEEAGKLLGHSLDLVFSIHADKEDGSPLESAVPPPFPGPCTLGTGLARYADLLNPPRKSALVALAAYATEPSEAEKLKHLTSPDGKDEYSQWIVASQRSLLEVMAAFPSAKPPLGVFFAAIAPRLQPRYYSISSSPRLAPSRVHVTSALVYGPTPTGRIHKGVCSTWMKNAVPAEKSHECSGAPIFIRASNFKLPSNPSTPIVMVGPGTGLAPFRGFLQERMALKEDGEELGSSLLFFGCRNRQMDFIYEDELNNFVDQGVISELIMAFSREGAQKEYVQHKMMEKAAQVWDLIKEEGYLYVCGDAKGMARDVHRTLHTIVQEQEGVSSSEAEAIVKKLQTEGRYLRDVW

SEQ ID NO: 37 - protein, 712 aa, Arabidopsis thaliana
MSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKVDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 38 - protein, 658 aa, synthetic construct
  REGION 1..658: note = CPR chim rique
MSPFGIDNTDFTVLAGLVLAVLLYVKRNSIKELLMSDDGDITAVSSGNRDIAQVVTENNKNYLVLYASQTGTAEDYAKKFSKELVAKFNLNVMCADVENYDFESLNDVPVIVSIFISTYGEGDFPDGAVNFEDFICNAEAGALSNLRYNMFGLGNSTYEFFNGAAKKAEKHLSAAGAIRLGKLGEADDGAGTTDEDYMAWKDSILEVLKDELGVEATGEESSIRQYELVVHTDIDAAKVYMGEMGRLKSYENQKPPFDAKNPFLAAVTTNRKLNQGTERHLMHLELDISDSKIRYESGDHVAVYPANDSALVNQLGKILGADLDVVMSLNNLDEESNKKHPFPCPTSYRTALTYYLDITNPPRTNVLYELAQYASEPSEQELLRKMASSSGEGKELYLSWVVEARRHILAILQDCPSLRPPIDHLCELLPRLQARYYSIASSSKVHPNSVHICAVVVEYETKAGRINKGVATNWLRAKEPAGENGGRALVPMFVRKSQFRLPFKATTPVIMVGPGTGVAPFIGFIQERAWLRQQGKEVGETLLYYGCRRSDEDYLYREELAQFHRDGALTQLNVAFSREQSHKVYVQHLLKQDREHLWKLIEGGAHIYVCGDARNMARDVQNTFYDIVAELGAMEHAQAVDYIKKLMTKGRYSLDVWS

SEQ ID NO: 39 - protein, 171 aa, Oryza meyeriana var. granulata
MSDATSSSSSQPGSGGGAATAKTKELDAALYALGFEIEEVSPAEVTGRLLVTPTCCQPFKVLHGGVSALIAEGLASMGAHMASGYSRVAGVQLSINHFRSAALGDTVLVRAAPVHVGRSTQVWEVKLWKMDPSTKEKGAQISESRVTLLCNLPVPDSVKDAGEALKKYSKL

SEQ ID NO: 40 - protein, 247 aa, Panicum virgatum
MSAAGGDTIARVHAGIDSSNKTLLKSEALYKYVLDTSVLPHEPECMRELRLVTDKHEWGLMQSSPDEAQLLRMLIKLMGARNTIEVGVFTGYSLLATALALPADGRVIAIDVDREYYEIGRPFIEKAGVAGKVDFREGPALDHLDALLADERNLGAFDFAFVDADKPNYVRYHEQLLRLVRVGGTIVYDNTLWAGTVALPPDTPLSDLDRRFSAAIRDLNARLSADDRVEVCQLAIADGVTICRRLV

SEQ ID NO: 41 - protein, 363 aa, Rauvolfia serpentina
MSGSAAPEQSNSMAGDEEACLFAMQLASASVLPMVLKSAIELDLLELIKKAGQDAYVSPSELAAQLPTENPDAPVMLDRILRLLASYSVLNCTLRNLPDGGVERLYGLAPVCKFLTKNEDGVSMAALLLMNQDKILMESWYHLKDAVLEGGIPFNKAYGMTAFEYHGTDPRFNKVFNQGMSNHSTIIMKKILEIYNGFEGLKTVVDVGGGTGATLSMIVSKYPAIKGINFDLPHVTGDAPTYPGIEHVGGDMFFSVPKGDAIFMKWICHDWSDEHCLKFLKNCYQALPENGKVILAECLLPEAPDATLSTQNTVHVDVIMLAHNPGGKERTEKEFEALAKEAGFKGFRKICCAYNSWIMELLK

SEQ ID NO: 42 - protein, 510 aa, Vigna angularis
MSALLLILPLSLITVWVAYTLYQRLSFKLPPGPRPWPVVGNLYDIKPVRFRCFAEWAQSYGPIISVWFGSTLNVIVSNSELAKEVLKEQDQHLADRHRSRSAAKFSRDGKDLIWADYGPHYVKVRKVCTLELFSPKRLEALRPIREDEVTAMVESIFHHCTTTENLGKGILVRKFLGEVAFNNITRLAFGKRFVNSEGVLEEQGVEFKGVVENGLKLGASLAMAEHIPWLRWMFPLEEEAFAKHGARRDRLTRAIMEEHTQARKKSGGAKQHFVDALLTLQDKYDLSEDTIIGLLWDMITAGTDTSAISVEWAMAELIRNPRVQQKAQEELDRVIGLERVMTEADFLSLPYLQCVAKESLRLHPPTPLMLPHRANANVKVGGYDIPKGSNVHVNVWAVARDPAVWKDPLEFRPERFLEEDVDMKGHDFRLLPFGAGRRVCPGAQLGINLVTSMLGHLLHHFSWNPAEGVKPEEIDMGENPGLVTYMRTPLKGVPSPRLPSHLYKRLPADI

SEQ ID NO: 43 - protein, 510 aa, Glycine max
MSALLLIVPISLVTLWLGYTLYQRLRFKLPPGPRPWPVVGNLYDIKPVRFRCFAEWAQSYGPIISVWFGSTLNVIVSNSELAKEVLKEHDQQLADRHRSRSAAKFSRDGKDLIWADYGPHYVKVRKVCTLELFTPKRLESLRPIREDEVTTMVESVYNHCTTTGNLGKAILVRKHLGSVAFNNITRLAFGKRFVNSEGVMDEQGVEFKAIVENGLKLGASLAMAEHIPWLRWMFPLEEGAFAKHGARRDRLTRAIMTEHTEARKKSGGAKQHFVDALLTLQDKYDLSEDTIIGLLWDMITAGMDTTAISVEWAMAELIRNPRVQQKVQEELDRVIGLERVMTEADFSSLPYLQCVIKEAMRLHPPTPLMLPHRANANVKVGGYDIPKGSNVHVNVWAVARDPAVWKDPLEFRPERFLEEDVDMKGHDFRLLPFGAGRRVCPGAQLGINLVTSMLGHLLHHFCWTPPEGMKPEEIDMGENPGLVTYMRTPIQALASPRLPSHLYKRVPAEI

SEQ ID NO: 44 - protein, 509 aa, Jatropha curcas
MSALLLISLSILFIFLAYKLYHHLRFKLPPGPRPWPIVGNLYDIKPVRFRCFAEWAQSYGPIISVWFGSTLNVIVSNTELAREVLKENDQQLADRHRSRSAAKFSRDGKDLIWADYGPHYVKVRKVCTLELFTPKRLEALRPIREDEVTAMVESIFTDCTNPENNGKSLLVKKYLGAVAFNNITRLAFGKRFVNSEGIMDEQGQEFKAIVANGLKLGASLAMAEHIPWLRWMFPLEEEAFAKHGARRDRLTRAIMEEHTLARQKSGGAKQHFVDALLTLQEKYDLSEDTIIGLLWDMITAGMDTTAITVEWAMAELIKNPRVQQKAQEELDRVIGFERVMTEADFSGLPYLQCIAKEGMRLHPPTPLMLPHRANSNVKIGGYDIPKGSNVHVNVWAVARDPAVWKNPEEFRPERFLEEDVDMKGHDFRLLPFGAGRRVCPGAQLGINLVTSMLGHLLHHFRWTPPEGVKPEEIDMSENPGLVTYMRTPLQAVATPRLPSELYKRLAVDM

SEQ ID NO: 45 - protein, 509 aa, Acacia koa
MSALLLIFISVIGVFLCYQLYQRLRFKLPPGPRPWPVVGNLYDIKPVRFRCFAEWAQSYGPIISVWFGSTLNVIVSNSELAREVLKDHDQQLADRHRSRSAAKFSRDGKDLIWADYGPHYVKVRKVCTLELFSPKRLEALRPIREDEVTAMVESIYRDSTNPENEGKSVMVKQYLGTVAFNNITRLAFGKRFVNSEGIMDEQGVEFKAVVTNGLKLGASLAMAEHIPWLRWMFPLEEGAFAKHGARRDRLTRAIMEEHTQARQRSGGAKQHFVDALLTLQDKYDLSEDTIIGLLWDMITAGMDTTAISVEWAMAELIKNPRVQQKAQEELDRVIGFERIITETDFSSLPYLQCVAKEAMRLHPPTPLMLPHRANANVKIGGYDIPKGSNVHVNVWAVARDPAVWKDPSEFRPERFLEEDVDMKGHDFRLLPFGAGRRVCPGAQLGINLVTSMLGHLLHHFCWTPPGGIKPEEIDMSENPGLVTFMRTPLQAVATPRLPSHLYKRVPAEI

SEQ ID NO: 46 - protein, 509 aa, Populus tomentosa
MSNLLLIPISFITILLTYKIYQRLRFKLPPGPRPWPIVGNLYDVKPVRFRCFAEWAQAYGPIISVWFGSTLNVIVSNTELAKEVLKENDQQLADRHRSRSAAKFSRDGKDLIWADYGPHYVKVRKVCTLELFSPKRLEALRPIREDEVTAMVESIFNDCTHPENNGKTLMVKKYLGAVAFNNITRLAFGKRFQNAEGVMDEQGLEFKAIVSTGLKLGASLAMAEHIPWLRWMFPLEEDAFAKHGARRDRLTRAIMDEHTLARQTSGGAKQHFVDALLTLQEKYDLSEDTIIGLLWDMITAGMDTTAISVEWAMAELIKNPRVQQKAQEEVDSVVGFERVMTEADFSGLPYLQCVAKEALRLHPPTPLMLPHRANANVKVGGYDIPKGSNVHVNVWAVARDPAAWKNPLEFRPERFLEEDVDMKGHDFRLLPFGAGRRVCPGAQLGINLVTSMLGHLLHHFCWTPPEGMKPEEIDMSENPGLVTYMRTPLQAVATPRLPSHLYKRVAVDI

SEQ ID NO: 47 - protein, 509 aa, Populus alba x Populus grandidentata
MSNLLLIPISFITILLTYKIYQRLRFKLPPGPRPWPIVGNLYDVKPVRFRCFAEWAQAYGPIISVWFGSTLNVIVSNTELAKEVLKENDQQLADRHRSRSAAKFSRDGKDLIWADYGPHYVKVRKVCTLELFSPKRLEALRPIREDEVTAMVESIFNDCTNPENNGKTLMVKKYLGAVAFNNITRLAFGKRFENAEGVMDEQGLEFKAIVSNGLKLGASLAMAEHIPWLRWMFPLEEDAFAKHGARRDRLTRAIMDEHTLARQTSGGAKQHFVDALLTLQEKYDLSEDTIIGLLWDMITAGMDTTAISVEWAMAELIKNPRVQQKAQEELDSVVGLERVMTEADFSGLPYLLCVAKEALRLHPPTPLMLPHRANANVKVGGYDIPKGSNVHVNVWAVARDPAAWKNPLEFRPERFLEEDVDMKGHDFRLLPFGAGRRVCPGAQLGINLVTSMLGHLLHHFCWTPPEGVKPEEIDMSENPGLVTYMRTPLQAVATPRLPSHLYKRVAVDI

SEQ ID NO: 48 - protein, 513 aa, Triticum turgidum subsp. durum
MSDVASLLPFAIALVAIPISLALLDRLRLGRLPPGPRPWPVLGNLRQIKPVRCRCFQEWAERYGPIISVWFGSSLTVVVSTSELAKEVLKEHDQQLADRPRNRSTQRFSRNGQDLIWADYGPHYIKVRKLCNLELFTQKRLEALRPIREDEVTAMVESVHRAAAGPGNEGKPLVVRNHLAMVSFNNITRLAFGKRFMNANGDIDEEGQEFKIIVNNGIKIGASLSVAEYIWYLRWLCPLNEELYKTHNERRDRLTKKIIEEHAQALQERGAKQHFVDALFTLREKYDLSDDTVFGLLWDMITAGMDTTVISVEWAMAELVRNPRVQKKLQEELDSVVGRDRVMSETDFQNLPYLMAVVKESLRLHPPTPLMLPHKASASVKVGGYSIPKGANVMVNVWAVARDPKVWSSPLEFRPERFLEESIDIKGSDFRVLPFGAGRRVCPGAQLGINLVASMIGHMLHHFEWSLPEGARPEDISMMESPGLVTFMGTPLQAVATPRLENEELYKRVPVEI

SEQ ID NO: 49 - protein, 510 aa, Salvia miltiorrhiza
MSAPLALLLLSLPVFFLLHNLYYRLRFRLPPGPRPWPIVGNLYDVKPLQFRCFADWAHSYGPIISVWFGSTLNVVVSSTELAKEVLKEKDQQLADRYRSRTATRLTKYGQDLIWADYGPHYVKVRKVCTVALFSAKSLESLRPIREDEVSAMVESIYNDCTAPGNSSKSLLLRKYLGAVSFNNITRIAFGKRYVDTEGRIHKQGEEMRSIADNRLRLGASRAIAEHIPWLRWMFPLNEEEFAKHAARRDRLTREIMEDHNIARQKSGGAKQHFCDALLTLKEKHDLTDDTIIGLLWDMIHAGMDTTAISVEWAMAELIRNPRVLQKVQEELDRVIGNERVVTELDFANLPYLRCVAKESLRLHPPTPLMLPHRASTNVKIGGYDIPKGSTVRVNVWAVARDPEVWKNPLEFRPERFLEDDVDIKGHDFRLLPFGAGRRICPGAQLGLDMVTSMLGRLLHHFKWAPPSGVSPEAINIAERPGVVTFMGTPLEAVATPRLPANLYERVAVDI

SEQ ID NO: 50 - protein, 513 aa, Cosmos sulphureus
MSTILPLLLYPSLTALLLYVLLNLRPRHPNRLPPGPSPWPIVGNLPHLGASPHQSLATLAAKYGPLMYLRLGFVDVVVAASASVAAQFLKVHDLNFASRPLSSGGKYIAYNYQDMVFAPYGPRWRMLRKICSVHMFSAKAMDGFRHVRQEEVAILTRTLVSAGKSPVKLGQILNVCTTNALARVVLGRRVFADGSAGGDPKADEFKDMVVELMVLAGEFHIGDFIPALDWLDLQGIKNKMKKLHARFDSFLHGILEEHKSGKFGAPSHGDLLSTLISLKDDADGEGGKLSDVEIKALLLNLFVAGTDTSSSTVEWAIAELIRHPKLLKQAQKEMDNVVGRDRLVTELDLNELNFLQAIVKETFRLHPSTPLSLPRIASESCEVDGYYIPKGSTLLVNVWAIARDPNVWADPLEFRPMRFLPGGEKPNVDVQGNNFEVIPFGAGRRICVGISLGLRMVQLLVATLVQTFDWELANGLNPEKLNMDEAFGLTLQKAEPLMVHPMPRLAPHVYGSH

SEQ ID NO: 51 - protein, 509 aa, Trifolium pratense
MSALFLTIPLSLITIFLFYTLFQRLRFKLPPGPRPWPVVGNLYDIKPVRFRCFAEWAQSYGPIISVWFGSTLNVIISNSELAKEVLKEKDQQLADRHRSRSAAKFSRDGQDLIWADYGPHYVKVRKVCTLELFSPKRIEALRPIREDEVTAMVESIFNDSTNSENLGKGILMRKYIGAVAFNNITRLAFGKRFVNSEGVMDEQGVEFKAIVANGLKLGASLAMAEHIPWLRWMFPLEEEAFAKHGARRDRLTRAIMDEHTQARQKSGGAKQHFVDALLTLQDKYDLSEDTIIGLLWDMITAGMDTTAISVEWAMAELIKNPRVQKKAQEELDKVIGFERVMTETDFSSLPYLQSVAKEALRLHPPTPLMLPHRANANVKIGGYDIPKGSNVHVNVWAVARDPAVWKNATEFRPERFLEEDVDMKGHDFRLLPFGAGRRVCPGAQLGINMVTSMLGHLLHHFCWAAPEGVNPEDIDMTENPGMVTYMRTPLQVVASPRLPSELYKRVPAD

SEQ ID NO: 52 - protein, 507 aa, Lonicera japonica
MSAPLLLLLLFLPILLYFYKTLRFKLPPGPRPWPIVGNLYQIKPVRFRCFSDWAQSYGPIISVWFGSTLNVIVSNTELAKEVLKEKDQQLADRHRSRSAAKFSRDGQDLIWADYGPHYVKVRKVCTLELFTPKRLEALRPIREDEVTAMVESIYNDCTNPDMRGKSLLVKKYLGAVAFNNITRLAFGKRFMNSEGVIDEQGQEFKAVVANGLKLGASLAMAEHIPWLRWFFPLEEEAFAKHGARRDRLTRAIMEEHTLARQKSGGAKQHFVDALLTLKDQYDLSEDTIIGLLWDMITAGMDTTAISVEWAMAELIKNPRVQQKAQEELDRVIGSERVLTEPDFANLPYLQSVAKEALRLHPPTPLMLPHRANANVKIGGYDIPKGSNIHVNVWAVARDPAVWKNTSEFRPERFLEEDVDMKGHDFRLLPFGAGRRVCPGAQLGINLVTSMLGHLLHHFSWAPPNGLKPEEIDMSENPGLVTYMRTPVQAVPNPRLPGHLYKRVAVDM

SEQ ID NO: 53 - protein, 509 aa, Nyssa sinensis
MSALLVLLLIFTFIFLTYKLYQRLRFKLPPGPRPWPIVGNLYDIKPVRFRCFAEWSQSYGPIISVWFGSTLNVIVSNTELAKEVLKEHDQHLADRHRSRSAAKFSREGKDLIWADYGPHYVKVRKVCTLELFSPKRLEALRPIREDEVTAMVESIYKDCTNSDGMGKSLLVKKYLGTVAFNNITRLAFGKRFVNAEGVVDEQGVEFKEIVSNGLKLGASLAMAEHIPWLRPMFPLEEEAFAKHGARRDRLTRAIMEEHTLARHRTGGAKQHFVDALLTLQGKYDLSEDTIIGLLWDMITAGMDTTAISVEWAMAELIKNPRVQQKAQDELDRVIGFERVLTEPDFSNLPYLQCVAKEALRLHPPTPLMLPHRANANVKIGGYDIPKGSNVHVNVWAVARDPAIWKNPLEFRPERFLEEDVDMKGHDFRLLPFGAGRRVCPGAQLGINLVTSMLGHLLHHFSWAPANGLKPEEIDMAENPGLVTYMRTPLQAVPTPRLPAHLYKRVAVDM

SEQ ID NO: 54 - protein, 514 aa, Pyrus ussuriensis x Pyrus communis
MSALSGGLLLPISSIIILISLAYKLYQRLRFKLPPGPRAWPIVGNLYDIKPVRFRCYAEWAQEYGPIISVWTGSTLNVVVSSSELAKEVLKEHDQKLADRHRSRSAAKFSKDGQDLIWADYGPHYVKVRKVCTLELFSPKRIEALRPIREDEVTAMVESIFKHCANPENYGKSLIVKKYLGAVAFNNITRLAFGKRFVNSEDVIDEQGLEFKAIVANGLKLGASLAMAEHIPWLRWMFPLEEEAFAKHGARRDRLTRAIMEEHTQARNKSGGAKQHFVDALLTLQDKYDLSEDTIIGLLWDMITAGMDTTAISVEWAMAELIKNPRVQQKAQEELDGVIGVERVMTEVDFSSLPYLQCVAKEALRLHPPTPLMLPHRANANVNIGGYDIPKGSNVHVNVWAVARDPAVWKDPNEFRPERFLEEDVDMKGHDFRLLPFGAGRRVCPGAQLGVNLVTSMLGHLLHHFNWTPTEGAKPEEIDMSENPGLVTYMRTPLQAVPTPRLPSQLYKRVPADI

SEQ ID NO: 55 - protein, 510 aa, Eucalyptus grandis
MSDSPSWTAYAAAWLATLALLLLSRRLRRRRYNLPPGPKPWPIIGNLNLIGSLPHRSLHELTKTYGPLMQVQFGSFPVVVGSSVEMARAFLKTHDVTFSGRPKTAAGKYTTYNYSDITWSPYGPYWRQARKLCIMELFSAKRLESYEYIRTEELMSLLGSLYVSRGAQLTLKDHLTDLSLNVISRMVLGKKYTEETENSIVKPDEFKKMLDELFLLNGALNIGDSIPWLDFLDLQGYIKRMKALSKKFDRFLEHVLDEHDERRRGVPNYVAKDMVDVLLQFADDPNLDVKLERKGVKGFTQDLIAGGTESSAVTVEWAISELLKRPEIFKKATEELDRVIGRDRWVSEKDIANLPYIDAIAKETMRLHPVAPMLVPRMAREDCNINGYDIPKGTRILVLVWTIGRDPKIWDAPEEFRPERFLEHDFDVKGQSFELLPFGSGRRMCPGYSLGLKVIQASLANLLHGFNWKLPEGMSNEDLDMEEIFGLSTPKKIPLVAVAEPRLPEHMYSI

SEQ ID NO: 56 - protein, 509 aa, Gossypium raimondii
MSELQISLFQVLITFFLVLFMAAITSLRKSKARNLTQGLIPGPRKLPLIGNLHQLAGPGLPHHTLRDLATKYGAIMHLQLGQISTVVVSSAEMAKEIMKTHDIVFANRPVLASAKIITYGCTDIAFSPYGNYWRNLRKICTSELLNATRVASFQSIREEEVLNLVETIKPNEGSAVNLSHKVFSLSYGITARAAFGKKCKDQEAFISVVTEETKVNSGFFVSEFFPSLKFLDTVLGLKHKVEKIHGEADMILGNIVNDHKESRSKGRSKDENKENLVDILLRIQEDGEFPLTDNNVKAVILDIFSAGSETSAGAVEWALSEMIKNPRVMTKAQAEVRQVFQGKGNVDETGIHQLKYLKCVIKETLRLHPVIPLLIPRESMKNCVVNGFEIPAKTRVIVNAWAIGRDPNHWVEPEKFEPERFVNSSVDFIGTNFEFIPFGAGRRVCPGILFALPTVELPLAQLLFHFDWKLPRGMKQEDIDMTEVFGVSVRRKNDLVLVPSLYRASTTVA

SEQ ID NO: 57 - protein, 507 aa, Zostera marina
MSDPTTTSLLLAIPPLLVVGITYLIYFRRRLPPGPRPWPVVGNLFSIKPVRFRCFAEWAAKYGPIISVWIGPTLNVVVSNSELAREVLKEKDQALANRHRTRSAARFSRDGQDLIWADYGPHYVKVRKVCTLELFSPKRLEGLRPIREDEVTAMVESLYKDSGKKMLVLKTYLSAVAFNNITRIAFGKRFMDEEGVVDKQGMELKAITVNGFKLGASLAMAENIPWLRCFFPLEEKTFAQHGANRDRFTKLIMDEHTTAREKSGGAKQHFVDALLTLKEKYDLSDDTIIGLLWDMITAGMDTTAISVEWAMAEVIRNPTVQIKAQEELDRVVGFERIMTEEDFPNLHYLQCVVKEALRLHPPTPLMLPHKAADDVSLGGYHIPKGSNVHVNVWAVARDPNEWKDPLRFSPERFLENNVDMKGHDFRLLPFGAGRRVCPGAQLGINMVQSMLGHLLHHFKWAPPEGVCSENLNMSENPGLVTFMQTPLQAIATPRLPAHLYKRIPYEM

SEQ ID NO: 58 - protein, 516 aa, Aquilegia coerulea
MSALLLLLTSLAIFFFIFIFLSHSIIQRLRFELPPGPKPKPIVGNLYDIQPVRFRYFLELSKTYGPIISVWIGSTLNVVVMNWELAKQVLKDKDQQLADRYRNRSSERFSKGGQDLIWADYGPHYVKVRKVCTVELFTPKRLEALKPIREDEVTAMVESIFKDCSSPVLSNADNYGKSIKVRDYLAPVAFNNITRLSFGKRFVNSEGLSDEQGLELKQIVDNGVKIGASLSIAEHIPWLRWMFPVDQAAYDSHAARRDRLTRAIMEEHTLARQQNGAKQHFVDALLTLQEKYDLSDDTIIGLLWDMITAGMDTTVITTEWAMAELVRNPRVQEKAQEELDRVIGIDRVMTEADISNLHYLQCVAKEALRLHPPTPLMLPHKANANVKIGGYDIPKGSNIHVNVWAIGRDPAVWKEPLEFRPERFMEEDIDMKGHDFRLLPFGAGRRVCPGAQLAITLVTSMLGHLLHHFSWTPPPGVKPEEMDMTENPGLVTYMRTPVQAIPTRRLPSQLYKRLPA

SEQ ID NO: 59 - protein, 511 aa, Actinidia chinensis var. chinensis
MSGLPLVLGTFTIIFLGLYYSYQKLRFKLPPGPRAWPVVGNLYDIKPVRFRCYAEWAQRYGPIMSVWFGSTLNVVVSNSELAKQVLKEKDQQLADRHRSRSAAKFSREGQDLIWADYGPHYVKVRKVCTLELFTPKRLESLRPIREDEVTAMVESIYIECTTPENKEKSLVVKKHIGAVAFNNITRLAFGKRFVNADGVMDEQGLEFKAIVANGLKLGASLAMAEHIPWLRWMFPLEEEAFAKHGAHRDRLTREIMDEHTLVRQKSSGAAKHHFVDALLTLQDKYDLSEDTIIGLLWDMITAGMDTTAITVEWGMAELIKNPRVQEKAQEELDRVIGFDRVMTELDFPNLPYLQCVAKETMRLHPPTPLMLPHRANSNVKIGGYDIPKGSNVHVNVWAIARDPAIWKDPLVFRPERFMEEDVDMKGHDFRLLPFGAGRRVCPGAQLGINLVTSMLGHLLHHFSWAPPNGLKPEEIDMTENPGLVTYMRNPLQAFPTPRLPAHLYKRVPVDV

SEQ ID NO: 60 - protein, 510 aa, Medicago truncatula
MSALFLTIPLSFIAIFLFYTLFQRLRFKLPPGPRPWPVVGNLYDIKPVRFRCFAEWAQSYGPIISVWFGSTLNVIVSNSKLAKEVLKENDQQLADRHRSRSAAKFSRDGQDLIWADYGPHYVKVRKVCTLELFSPKRIEALRPIREDEVTAMVESIFNDSTNSENLGKGILMRKYIGAVAFNNITRLAFGKRFVNSEGVMDEQGVEFKAIVANGLKLGASLAMAEHIPWLRWMFPLEEEAFAKHGARRDRLTRAIMEEHTQARQKSGGAKQHFVDALLTLQEKYDLSEDTIIGLLWDMITAGMDTTAISVEWAMAELIKNPRVQQKAQEELDKVIGFERVMTETDFSSLPYLQCVAKEALRLHPPTPLMLPHRANTNVKIGGYDIPKGSNVHVNVWAVARDPAVWKDATEFRPERFLEEDVDMKGHDFRLLPFGAGRRVCPGAQLGINMVTSMLGHLLHHFCWAPPEGVNPAEIDMAENPGMVTYMRTPLQVVASPRLPSELYKRVTADI

SEQ ID NO: 61 - protein, 512 aa, Malus baccata
MSFVLIFFTVVLAFFLYRLFAPGGSRHAQPLPPGPKPWPVVGNLPHLGPVPHHSLAALARQYGPLMHLRLGFVDVVVAASASVASQFLKTHDANFSSRPPNSGAKHLAYNYQDLVFAPYGPRWRMLRKISSVHLFSGKALDDLKHVRQEEVGVLAHGLASAGSKPVNLGQLLNVCTVNALGRVMVGRRLFGDGGGREDQKADEFKSMVVEMMVLAGVFNISDFIPALEWLDLQGVAGKMKKLHKRFDAFLTAIVEDHKRSGEGKHVDMLTTLLSLTDDADGEGAKLTDTEIKALLLNMFTAGTDTSSSTVEWAIAELIRHPKILAQLQQELDQVVGRDRLVTESDLPNLTYLQAVIKEIFRLHPSTPLSLPRMASESCEINGFHIPKGATLLVNVWAISRDPAQWSEPLEFRPERFLPGGEKPSVDVKGNDFEVIPFGAGRRICAGMTLGLRMVSLMTATLVHGFDWTLADGLTPEKLNMDEAYGLTLQRAAPLMVHPRNRLAPHAYNASSP

SEQ ID NO: 62 - protein, 524 aa, Ricinus communis
MSESAAHQSYFHMFLAMEQQILSFPVLLSFLLFIFMVLKVWKKNKDNPNSPPGPRKLPIIGNMHQLAGSDLPHHPVTELSKTYGPIMSIQLGQISAIVISSVEGAKEVLKTQGELFAERPLLLAAEAVLYNRMDIIFGAYGDHWRQLRKLCTLEVLSAKRIQSFSSLRQEELSHFVRFVHSKAGSPINLSKVLFALTNSIIARIATGKKCKNQDALLDLIEDVIEVSGGFSIADLFPSLKFIHVITGMKSRLEKLHRITDQVLEDIVNEHKATRAASKNGGGDDDKKEAKNLLDVLLDLQEDGSLLQVPLTDDSIKAAILEMLGGGSDTSAKTTEWAMSEMMRYPETMKKAQEEVRQAFGNAGKIDEARIHELKYLRAVFKETLRLHPPLAMIPRECRQKTKINGYDIYPKTKTLINVYAIGRDPNVWSEPEKFYPERHLDSPIDFRGSNFELIPFGAGKRICPGMTLAITTVELFLAHLLYYFDWKFVDGMTADTLDMTESFGASIKRKIDLALVPIPVSPLP

SEQ ID NO: 63 - protein, 518 aa, Sorghum bicolor
MSHVPLLLGSLAVSVVVWCLLLRRGGDGKGKGNGKRPLPPGPRGWPVLGNLPQVGSHPHHTMYALAKEYGPLFRLRFGSADVVVAASARVAVQFLRAHDANFSNRPPNSGAEHMAYNYQDMVFAPYGSRWRALRKLCALHLFSAKALDDLRGVREGEVALMVRQLALHQHQHAGVPLGQVANVCATNTLARATVGRRVFAVDGGEEAREFKDMVVELMQLAGVFNVGDFVPALAWLDLQGVVGKMKRLHRRYDDMMNSIIRKRKAAEEGKDLLSVLLARMREQQSLADGEDSRINETGIKALLLDLFTAGTDTTSSTVEWALAELIRHPDVLKKAQEELDAVVGRDRLVSETDLPRLTYLTAVIKETFRLHPSTPLSLPRVAAEECEVDGFRIPAGTTLLVNVWAIARDPEAWPEPLQFRPDRFLPGGSHAGVDVKGSDFELIPFGAGRRICAGLSWGLRMVTLMTATLVHALDWDLADGMTADKLDMEEAYGLTLQRAVPLMVRPTPRLLPSAYAAE

SEQ ID NO: 64 - protein, 513 aa, Populus euphratica
MSEITSTWVSYAAPLLATITLILLGRLIRRRKLHLPPGPKPWPIIGNLDLMGELPHRSLEALSKKYGSLMQVKFGSHPVVVGSSVEMARAILKTHDLSLAGRPKTASGKYTTYNYQNITWAQYGPYWRQARKLCLMELFSPKRLDQFEYIRVEENLKFLNTLFQKRGKPVSVRDHFSDLSFSVISRLVLGRKYMAESEDEKDMLSLKEFKEVLDEMFLLNGVLVIGDFIPWLAFLDLQGYIKRMKAVAKKMDMFMEHALEEHHARRKGLKDYEPRDMLDILLQVADDPNLEVKLDRIGVKAFTQDLINGGTESSAVTTEWALAEIMKKPEIFDKATEELDRVIGRERWVQENDIDNLPFINAIVKETMRLHPVAPLLVPRLAREDIQIAGYDIPKGTRVLVNASAIGRDPSIWDKPKEFCPERFIGKSVDVKGHDFELLPFGAGRRICPGYPLGLKVIQTSVANLLHGFKWKLPDNMTAKDLNMEEIFGLSAPRKVPLVAVLEPRLPSELYSL

SEQ ID NO: 65 - protein, 509 aa, Nicotiana tabacum
MSALYFIFISITLIFLAHKLYHRLRFKLPPGPRPLPVVGNLYDIKPVRFRCFADWAKTYGPIFSVYFGSQLNVVVTTAELAKEVLKENDQNLADRFRTRPANNLSRNGMDLIWADYGPHYVKVRKLCNLELFTPKRLEALRPIREDEVTAMVENIFKDCTKPDNTGKSLLMREYLGSVAFNNITRLTFGKRFMNSKGEIDEQGQEFKGIVSNGIKIGGKLPLAEYVPWLRWFFTMENEALVKHSARRDRLTRMIMDEHTLARKKTGDTKQHFVDALLTLQKQYDLSDDTVIGLLWDMITAGMDTTTITVEWAMAELVKNPRVQQKAQEELDRVIGTDRIMSETDFSKLPYLQCVAKEALRLHPPTPLMLPHKASASVKIGGYDIPKGSIVHVNVWAVARDPAVWKNPLEFRPERFLEEDVDMKGHDYRLLPFGAGRRVCPGAQLAINLVTSMLGHLLHHFTWAPPPGVNPENIDLEESPGTVTYMKNPIQAIPTPRLPAHLYGRVPVDM

SEQ ID NO: 66 - protein, 505 aa, Raphanus sativus
MSWFLITAAIAAVVSYKLIQRLRLNLPPGPLPKPIIGNLCDIKPVRFICYYEWAQTYGPIISVWIGSVLNVVVSSAELAKEVLKDHDQKLADRHRNRSTEAFSRNGQDLIWADYGPHYVKVKKVCMLELFTPKRLESLRPVREDEVNSMVESVFRDCNLPENIAKGIQLRKYLGAVAFNNITRLVFGKRFVNAEGVMDEQGLEFHAIVSNGLKLGASLSIAEHISWLRWMCPADEKAFAEHGARRDLLTRAIMEEHTLARQKSSGPKQHFVDALLTLKDQYDLSEDTIIGLLWDMITAGMDTAAITAEWAMAEMIKNPRVQQKVQEEFDRVIGLDRILTEPDLARLPYLKCIVKESFRLHPPTPLMVPHRSSADVKIGGYNIPKGSNVHVNVWAVGRDPAVWRNPLDFRPERFLEEDVDMKGHDFRLLPFGAGRRVCPGAQLGINLVTSMLSHLLHHFVWTLPQGTKPEEIDMSGNPGIVTFMRTPVQAVATPRLPSDLYKRVAC

SEQ ID NO: 67 - protein, 506 aa, Ipomoea nil
MSMMNTTAWIWAGFAAVLAAAIAVYELLFRNNKRKRLPPGPKGLPILGHLHLLGKNPHQDMQKLSKTYGPMMYLRLGLTDNIIVSSPQAAERFLKTHDLNFASRPPNQAANYIAYEQRDLAFGPYGPFWRNMRKLCTLELLSNLKINSFQSMRREELGLVVDSFKQAAQNGKAVDLSTTISSMSMDMSCRMVFGKTYKNQDFGEMGFKALIHEIVYLAAVPNLGDYFPFLGKLDVQGFTRRMKAVGKLTDQFMERIVEEHEEAASKGITNKTKDFVDTLLEIMKSANTDFQFSRKHVKAMMLDILVTSMDTSSTVIEWTMSELFRHPEIMKKVRDEVAEKVGYDRMVEEADLEGLEYLEMVVKESLRLHPVVPLLLPHIAMDDCVVDDFHVPKNTRVMVNAWAIGRDPNVWSNPEKFNPERFSKNRVDYRGRDFELIPFGSGRRSCPGLQLGITVVRHVVAQLVHCFDWTLPNKMLPEDLDMTESFGLVISRDKPLMAIPTYRLPQ

SEQ ID NO: 68 - protein, 719 aa, Citrus sinensis; identical to SEQ ID NO: 31

SEQ ID NO: 69 - protein, 717 aa, Arabidopsis thaliana
MDQIEAMLCGGGEKTKVAVTTKTLADPLNWGLAADQMKGSHLDEVKKMVEEYRRPVVNLGGETLTIGQVAAISTVGGSVKVELAETSRAGVKASSDWVMESMNKGTDSYGVTTGFGATSHRRTKNGTALQTELIRFLNAGIFGNTKETCHTLPQSATRAAMLVRVNTLLQGYSGIRFEILEAITSLLNHNISPSLPLRGTITASGDLVPLSYIAGLLTGRPNSKATGPDGESLTAKEAFEKAGISTGFFDLQPKEGLALVNGTAVGSGMASMVLFEANVQAVLAEVLSAIFAEVMSGKPEFTDHLTHRLKHHPGQIEAAAIMEHILDGSSYMKLAQKVHEMDPLQKPKQDRYALRTSPQWLGPQIEVIRQATKSIEREINSVNDNPLIDVSRNKAIHGGNFQGTPIGVSMDNTRLAIAAIGKLMFAQFSELVNDFYNNGLPSNLTASSNPSLDYGFKGAEIAMASYCSELQYLANPVTSHVQSAEQHNQDVNSLGLISSRKTSEAVDILKLMSTTFLVGICQAVDLRHLEENLRQTVKNTVSQVAKKVLTTGINGELHPSRFCEKDLLKVVDREQVFTYVDDPCSATYPLMQRLRQVIVDHALSNGETEKNAVTSIFQKIGAFEEELKAVLPKEVEAARAAYGNGTAPIPNRIKECRSYPLYRFVREELGTKLLTGEKVVSPGEEFDKVFTAMCEGKLIDPLMDCLKEWNGAPIPIC

SEQ ID NO: 70 - protein, 502 aa, Panicum virgatum
MSDLVLLEKALLGLFAAAVLAIAVAKLTGKRLRLPPGPAGAPVVGNWLQVGDDLNHRNLMDLAKRFGDIFLLRMGVRNLVVVSTPELAKEVLHTQGVEFGSRTRNVVFDIFTGKGQDMVFTVYGDHWRKMRRIMTVPFFTNKVVVQNRAGWEEEARLVVEDVRRDPRAAAEGVVIRKRLQLMMYNDMFRIMFDRRFESEQDSLFNKLKALNAERSRLSQSFEYNYGDFIPVLRPFLRGYLNRCHDLKTRRMKVLEDNFVQERKKVMAQTGEIRCAMDHILEAERKGEINHDNVLYIVENINVAAIETTLWSIEWGIAELVNHPTIQSKLREEITSVLGAGVAVTEPDLERLPYLQAVVKETLRLRMAIPLLVPHMNLNDGKLHGYDIPAESKILVNAWFLANDPKRWVKPEEFRPERFLEEEKAVEAHGNDFRFVPFGVGRRSCPGIILALPIIGITLGRLVQNFQLLPPPGQDKVDTTEKPGQFSNQIAKHATIVCKLLEA

SEQ ID NO: 71 - protein, 362 aa, Panicum virgatum
MSGSTATDVAAAADEEACMYALQLASSSILPMTLKNAIELGLLEVLQKDPAAALAPEEVVAQLPVAPANPDAAAMVDRMLRLLASYDVVRCQMEEGKDGRYSRRYAAAPVCKWLTPNEDGVSMAALALMNQDKVLMESWYYLKDAVLEGGIPFNKAYGMTAFEYHGTDPRFNRVFNEGMKNHSVIITKKLLEFYAGFEGVGTLVDVGGGVGATLHAITSRYPGIRGVNFDLPHVISEAPPFPGVEHVGGDMFKAVPAGDAILMKWILHDWSDAHCAAILKNCYDALPAGGKVIAVECILPVNPEATPKAQGVFHVDMIMLAHNPGGKERYEREFEELAKGAGFTGFKATYIYANAWAIEFTK

SEQ ID NO: 72 - protein, 364 aa, Arabidopsis thaliana
MSGSTAETQLTPVQVTDDEAALFAMQLASASVLPMALKSALELDLLEIMAKNGSPMSPTEIASKLPTKNPEAPVMLDRILRLLTSYSVLTCSNRKLSGDGVERIYGLGPVCKYLTKNEDGVSIAALCLMNQDKVLMESWYHLKDAILDGGIPFNKAYGMSAFEYHGTDPRFNKVFNNGMSNHSTITMKKILETYKGFEGLTSLVDVGGGIGATLKMIVSKYPNLKGINFDLPHVIEDAPSHPGIEHVGGDMFVSVPKGDAIFMKWICHDWSDEHCVKFLKNCYESLPEDGKVILAECILPETPDSSLSTKQVVHVDCIMLAHNPGGKERTEKEFEALAKASGFKGIKVVCDAFGVNLIELLKKL

SEQ ID NO: 73 - protein, 364 aa, Catharanthus roseus
MSGSANPDNKNSMTKEEEEACLSAMRLASASVLPMVLKSAIELDLLELIKKSGPGAYVSPSELAAQLPTQNPDAPVMLDRILRLLASYSVLNCTLKDLPDGGIERLYSLAPVCKFLTKNEDGVSMAALLLMNQDKVLMESWYHLKDAVLEGGIPFNKAYGMTAFEYHGKDPRFNKVFNQGMSNHSTIIMKKILEIYQGFQGLKTVVDVGGGTGATLNMIVSKYPSIKGINFDLPHVIEDAPSYPGVDHVGGDMFVSVPKGDAIFMKWICHDWSDAHCLKFLKNCHEALPENGKVILAECLLPEAPDSTLSTQNTVHVDVIMLAHNPGGKERTEKEFEALAKGAGFRGFIKVCCAYNSWIMELLK

SEQ ID NO: 74 - protein, 357 aa, Triticum aestivum
MSGSIAAGADEDACMYALQLVSSSILPMTLKNAIELGLLETLMAAGGKFLTPAEVAAKLPSAANPEAPDMVDRMLRLLASYNVVSCRTEDGKDGRLSRRYGAAPVCKYLTPNEDGVSMSALALMNQDKVLMESWYYLKDAVLDGGIPFNKAYGMSAFEYHGTDPRFNRVFNEGMKNHSIIITKKLLESYKGFEGLGTLVDVGGGVGATVAAITAHYPTIKGINFDLPHVISEAPPFPGVTHVGGDMFQKVPSGDAILMKWILHDWSDEHCATLLKNCYDALPAHGKVVLVECILPVNPEATPKAQGVFHVDMIMLAHNPGGRERYEREFEALAKGAGFAAMKTTYIYANAWAIEFTK

SEQ ID NO: 75 - protein, 354 aa, Triticum aestivum
MSGSTADEEACMFALQLGSSSILPMTLKNAIELGLLDTLVAADGKLLSPAELAAKLPSTANPAAPDMVDRMLRLLASYKVVSCTLEEDKDGRLSRRYGAEPVCKFLTPNEEGVSMAPLALMNQDKVLMESWYYLKDAVLDGGIPFNKAYGMSAFEYHGTDPRFNRVFNEGMKNHSIIITKKLLELYKGFNGVGTLVDVGGGIGATVGAITAHYPTIKGINFDLSHVISEAPPFPGVTHVGGDMFQKIPSGDTILMKWILHDWSDEHCATLLKNCYDALPTHGKVMLVECILPVNPEATPKAQGGFHLDMIMLAHNPGGKERYEREFEALAKGAGFGAMKTTYIYANTWVIEFTK

SEQ ID NO: 76 - protein, 362 aa, Panicum virgatum; identical to SEQ ID NO: 71

SEQ ID NO: 77 - protein, 366 aa, Nicotiana tabacum
MSESSTKSQIPTQSEEERNCTYAMQLLSSSVLPFVLHSTIQLEVFEILAKSNDTKLSASQIVSQIPNCKNPDAATMLDRMLYVLASYSLFTCSIVEDEENNGGQKRVYGLSQVGKFFVRDEDGASMGPLLALLQDKVFINSWFELKDAVLEGGVPFDRVHGVVHAFEYPKSDPKFNDVFNKAMINHTTVVMKKILENYKGFENLKTLVDVGGGLGVNLKMITSKYPTIKGTNFDLPHVVQHAPSYPGVEHVGGDMFESVPEGDAIFMKWILHDWSDSHNLKLLKNCYKALPDNGKVIVVEAILPVKPDIDTAVVGVSQCDLIMMAQNPGGKERSEEEFRALATEAGFKGVNLICCVCNFWVMEFCK

SEQ ID NO: 78 - protein, 365 aa, Picea abies
MSGSASENSEMNTKIVNEDEWLLGMELGNFSCLPMGMKAAIELDVLQIIANAGNGVQLSPRQIVAHIPTTNPDAAITLDRILRVLASHSVLSCSVTTDENGKAERLYGLTPLCKYLVKNQDGVSLAPLVLMNQDKVLMDSWYYLKDAVLEGSQPFTKAHGMNAFEYPAKDQRFNRVFNRGMSEHSTMLMNKILDTYQGFKEVQELVDVGGGVGSTLNLIVSRYPHISGINFDMPHVVTDAPHYPAVKHVGGDMFDSVPTGQAIFMKWILHDWSDDHCRKLLKNCHKALPEKGKVIVVDTILPVAAETSPYARQGFHIDLLMLAYNPGGKERTEQEFQDLAKEAGFAGGVKPVCCANGMWVMEFHK

SEQ ID NO: 79 - protein, 365 aa, Zea mays
MSGSTAGDVAAVVDEEACMYAMQLASSSILPMTLKNAIELGLLEVLQKEAGGGKAALAPEEVVARMPAAPGDPAAAAAMVDRMLRLLASYDVVRCQMEDRDGRYERRYSAAPVCKWLTPNEDGVSMAALALMNQDKVLMESWYYLKDAVLDGGIPFNKAYGMTAFEYHGTDSRFNRVFNEGMKNHSVIITKKLLDFYTGFEGVSTLVDVGGGVGATLHAITSRHPHISGVNFDLPHVISEAPPFPGVRHVGGDMFASVPAGDAILMKWILHDWSDAHCATLLKNCYDALPENGKVIVVECVLPVNTEATPKAQGVFHVDMIMLAHNPGGKERYEREFRELAKGAGFSGFKATYIYANAWAIEFIK

SEQ ID NO: 80 - protein, 366 aa, Stylosanthes humilis
MSGSTGETQITPTVVSDEEANLFAMQLASASVLPMVLKSAIELDVLEIIAKAGPGVHLSPADIAANVPTTNPDAAVMLDRMLRLLACYNILNHSLRTLPDGKVERLYGLAPVAKFLVKNEDGVSLSALNLMNHDKVLMESWYYLKETVLEGGIPFNKAYGMTAFEYHGTDPRFNKVFNKGMNDHTTITMKKILEIYTGFEGLKSLVDVGGGVGAVVNMIVTKYPHIKGINFDLPHVIEDAPSYPGVEHVGGDMFVSVPKADAVFMKWICHDWSDEHCIKFLKNCYDAVPDNGKVIVAECILPVAPDSSLATKGVVHIDVIMLAHNPGGKERTEKEFEALAKAAGFQGFRVVCCAFNTYIMEFLKKP

SEQ ID NO: 81 - protein, 363 aa, Saccharum officinarum
MSGSTAEDVAAVADEEACMYAMQLASASILPMTLKNALELGLLEVLQAEAPAGKALAPEEVVARLPVAPTNPDAADMVDRMLRLLASYDVVKCQMEDKDGKYERRYSAAPVGKWLTPNEDGVSMAALTLMNQDKVLMESWYYLKDAVLDGGIPFNKAYGMTAFEYHGTDPRFNRVFNEGMKNHSVIITKKLLEFYTGFEGVSTLVDVGGGIGATLHAITSHHPQIKGINFDLPHVISEAPPFPGVQHVGGDMFKSVPAGDAILMKWILHDWSDAHCATLLKNCYDALPENGKVIIVECVLPVNTEAVPKAQGVFHVDMIMLAHNPGGRERYEREFHDLAKGAGFSGFKATYIYANAWAIEFIK

SEQ ID NO: 82 - protein, 365 aa, Zea mays
MSGSTAGDVAAVVDEEACMYAMQLASSSILPMTLKNAIELGLLEVLQKEAGGGKAALAPEEVVARMPAAPSDPAAAAAMVDRMLRLLASYDVVRCQMEDRDGRYERRYSAAPVCKWLTPNEDGVSMAALALMNQDKVLMESWYYLKDAVLDGGIPFNKAYGMTAFEYHGTDARFNRVFNEGMKNHSVIITKKLLDFYTGFEGVSTLVDVGGGVGATLHAITSRHPHISGVNFDLPHVISEAPPFPGVRHVGGDMFASVPAGDAILMKWILHDWSDAHCATLLKNCYDALPENGKVIVVECVLPVNTEATPKAQGVFHVDMIMLAHNPGGKERYEREFRELAKGAGFSGFKATYIYANAWAIEFIK

SEQ ID NO: 83 - protein, 359 aa, Cucumis sativus
MSLGVSNDEEEAQLFAMQLASASVLPMVLKTAIELDLLEIIGRGGEGALLSPSQIASQLSGLKNPEAHVMLDRMLRLLASYSILTCSLDPLPDGSVQRLYGLAPVSKFLIKNHDGVSIAPLCLMNQDKVLMESWYHLKDAVLEGGIPFNRAYGMSAFEYHGTDPRFNKVFNKGMSDHSTITMKKILETYKGFEGLNSVVDVGGGTGAVLNMIVSKYPSIRGINFDLPHVIQDAPPYPGVEHVGGDMFVSVPKGDAIFMKWICHDWSDHHCLKFLKNCYDALPEHGKVIVAECILPLAPDASLATKGVIHIDLIMLAHNPGGKERTEKEFQALSKAAGFDGFKVHCCAFNTYVMEFLKTP

SEQ ID NO: 84 - protein, 367 aa, Tarenaya hassleriana
MSGSTAETQITPNQVSDDEANLFAMQLASASVLPMVLKSAIELDLLEIMAKNAGFSAQMSPSDIASHLPTKNPDAPVMLDRILRLLASYSILTCSVRTLSSGGVERLYGLGPVCKYLTKNEDGVSIAALCLMNQDKVLMESWYHLKDAVLEGGIPFNKAYGMTAFEYHGTDQRFNKVFNNGMSNHSTITMKKILETYNGFEGLSSVVDVGGGIGATLSMIVSKYPAIKGINFDLPHVIEDAPSYPGIEHVGGDMFVSVPKGDAIFMKWICHDWSDEHCLKFLKNCYEALPDNGKVIVAECILPVAPDPSLLTKQVVHIDCIMLAHNPGGKERTEKEFEALAKGSGFHGFQVVCNAFGTYIMEFLKKI

SEQ ID NO: 85 - protein, 366 aa, Ziziphus jujuba var. spinosa
MSGSTGETQMTPTQVSDEEANLFAMQLASASVLPMVLKSAIELDLLEIIAKAGPGAFVSPSDIASQLPTKNPDASVVLDRILRLLASYSILTYSLRTLPDGKVERLYGLGPVCKFLTKNEDGVSIAPLCLMNQDKVLMESWYYLKDAVLEGGIPFNKAYGMTAFEYHGTDPRFNKIFNKGMADHSTITMKKLLETYKGFEGLKTVVDVGGGTGAVLSMILSKYPSIKGINFDLPHVIEDAPAYPGVQHVGGDMFVSVPKGDAIFMKWICHDWSDEHCLKFLKNCYDALPENGKVILAECILPVAPDSSLAAKGVIHIDVIMLAHNPGGKERTEQEFEALAKGAGFQGFRVACNAFNTYVIEFLKKP

SEQ ID NO: 86 - protein, 375 aa, Cucurbita maxima
MSGSSGKGCEVQITPTTRVSDEEAQLFAMQLASASVLPMVLKAAIELDVLEIIGAAGEGALLSPSQIASHLSFSGAKTPNPEAGVVLDRMLRLLASYSILTYSLNTLPDGSLQRLYGLAPVAKFLVKNQDGVSIAPLCLMNQDKVLMESWYHLKDAVLEGGIPFNKAYGMTAFEYHGTDPRFNRVFNRGMSDHSTITMKKILETYKGFEGLNSVVDVGGGTGAVLNMIVSKYPSIRGINFDLPHVIQDAPAYPGVQHVGGDMFVSVPKGDAIFMKWICHDWSDQHCLKLLKNCHDSLPEHGKVIVAECILPVAPDSSLATKGVIHIDVIMLAHNPGGKERTEQEFHALAKAAGFQGFKVHSCAFNTYIMEFLKTL

SEQ ID NO: 87 - protein, 365 aa, Ipomoea nil
MSGSTGNDSPMTNRVDSDEEASLFAMQLASASVLPMVLKSAIELDLLELIAKAGPGAAVSPSELAAQLPTTNPDAAVMLDRILRLLCTYSVLNCSLRTLPDGRVERLYSLAPVCKYLTKNGDGVSMAPLLLMNQDKVLMESWYHLKDAVLEGGIPFNKAYGMTAFEYHGTDPRFNKVFNRGMSDHSTITMKKILDDYKGFEGLKSLVDVGGGTGATLNMILSKYPNIKGINFDLPHVIEDSPKYPGIEHVGGDMFVSVPKGDAIFMKWICHDWSDAHCLKFLKNCYDALPENGKVIVAECNLPESPDTSLATKNVVHIDVIMLAHNPGGKERTEKEFEALAMGSGFRHFRKACCAVNTWIMEFCK

SEQ ID NO: 88 - protein, 365 aa, Thalictrum tuberosum
MSGSTENNHNLTPAEEEEEEACLHAMQLASASVLPMVLKAAIELDVLEIIAKAGKGAYVAPSEIASQLSTSNSQAPTVLDRMLRLLASYKVLTCNLRNLEDGGVERLYGLAPVCKFLVKNEDGVSMAPLVLMNQDKVLMESWYHLKDAVLDGGIPFNKAYGMTAFEYHGTDPRFNKVFNRGMADHSTITMKKLLELYKGFEGLKSVVDVGGGTGATVNMIVTKHPTIKGINFDLPHVIDDAPAYPGVEHIGGDMFVSVPKGDAIFMKWILHDWSDEHSVKFLKNCYESIPADGKVIIVESVLPVFPETNLAAHTCFQLDNIMLAHNPGGKERTEKDFKALSVKAGFTGFKVVCGAFGSWVMEFCK

SEQ ID NO: 89 - protein, 371 aa, Punica granatum
MSGSTGQSAETQMTPTQVSDEEANLFAMQLASASVLPMVLKAALELDLLEIMAKSCPARGGYISSSDIAVQLPTINPEAPVTLDRVLRLLASYSVLTCKLRDLPDGKVERLYGLGPVCKFLTKNEDGVSIAPLCLMNQDKVLMESWYHLKDAILEGGIPFNKAYGMTAFEYHGTDPRFNKVFNRGMSDHSTITMKKILETYKGFEGLNTIVDVGGGTGAVLSMIVAKYPSIKGINFDLPHVIEDAPSLPGVEHVGGDMFVSVPKGDAIFMKWICHDWSDEHCLKFLKNCYAALPDHGKVIVAECILPLSPDPSLATKGVIHIDCIMLAHNPGGKERTEKEFEALAKGAGFEGFRVACCAFNTYVMEFLKKA

SEQ ID NO: 90 - protein, 365 aa, Brassica cretica
MSGSTEETQITPVQVTDDEAALFAMQLASASVLPMVLKTALDLDLLEIMAKNSSQMSPSEIASQLQTKNPEAPTMLDRILRLLTSYSILTCSSRAIPGGDSVERIYGLGPVCKYLTKNEDGVSIAALCLMNQDKVLMESWYHLKDAILDGGIPFNKAYGMTAFEYHGKDLRFNTVFNNGMSNHSTITMKKILETYKGFEGLTSLVDVGGGIGATLKMIVSKYPNLKGINFDLPHVIGEATSHPGIEHVGGDMFVSVPEGDAIFMKWICHDWSDEHCVKFLKNCYEALPEDGKVILAECILPETPDSSLSTKQVVHVDCIMLAHNPGGKERTEKEFERLAKESGFKGIKVACNAFGVYVIELLKKI

SEQ ID NO: 91 - protein, 362 aa, Lycium chinense
MSGSTSQNNNLTHTEDEDFLFAMQLASASVLPMVLKSVVELDLLEIMAKAGPGATISPSELAAQLPSKNPDAPVMLDRMLRLLATYSVLNCTLRTHPDGRVERLYSLAPVCKFLTKNADGVSVAPLLLMNQDKVLMESWYHLKDTVLDGGIPFNKAYGMTAFEYHGTDPRFNKVFNRGMSDHSTMTMKKILEDYKGFEGLNSIVDVGGGTGATVNMIVSKHPSIKGINFDLPHVIEDAPAYPGVEHVGGDMFVSVPKADAIFMKWICHDWSDDHCQKFLKNCYEALPANGKVIIAECLLPEAPDTSFSTKNTVHVDIIMLAHNPGGKERTEKEFEALAKGAGFSGFRKACCAYNTWVMEFNK

SEQ ID NO: 92 - protein, 366 aa, Acer yangbiense
MSGSSGETQMTPTQISDEDANLFAMQLTSASVLPMVLKSALELDLLEIIAKAGPGAHLSPTQVASQLPTTNPDAPVMLDRILRLLASYSILTCSLKTLPDGKVERLYGLAPVCKFLTKNEDGVSISALALMNQDKVLMESWYHLKDAVLEGGIPFNKAYGMTAFDYHGTDPRFNKVFNNGMSSHSSITMKKILETYKGFEGLKSVVDVGGGTGATVNQIVSTYPSIKGINFDLPHVIEDAPSYPGVEHVGGDMFVSVPKGDAIFMKWICHDWSDEHCLKFLKNSYEALPENGKVIVCECILPVTPDPSLGSKVVFHIDCIMLAHNPGGKERTEDDFKALAKGAGFKGFKVVCCAFNTYIMEFLKNA

(19) United States
(12) Patent Application Publication
(10) Pub. No.: US 2025/0019727 A1
(43) Pub. Date: Jan. 16, 2025

(54) METHOD FOR THE BIOSYNTHESIS OF CAFFEIC ACID AND FERULIC ACID

(71) Applicant: ABOLIS BIOTECHNOLOGIES, EVRY (FR)

(72) Inventors: CÉLIA ROUSSEL, SAINTRY SUR SEINE (FR); SABRINA VARLET, YERRES (FR); CLÉMENTINE FOJCIK, PALAISEAU (FR); CYRILLE PAUTHENIER, JUVISY-SUR-ORGE (FR)

(21) Appl. No.: 18/712,727

(22) PCT Filed: Nov. 23, 2022
(86) PCT No.: PCT/FR2022/052169

§ 371 Date: May 23, 2024

(30) Foreign Application Priority Data
Nov. 23, 2021 (FR) FR2112410

(51) Int. Cl. C12P 7/42; C12N 9/00; C12N 9/02; C12N 9/16

(52) U.S. Cl. CPC C12P 7/42; C12N 9/0071; C12N 9/16; C12N 9/93; C12Y 301/02002; C12Y 602/01012

(57) ABSTRACT

The present invention relates to a recombinant microorganism comprising a heterologous nucleic acid sequence coding for a 4-coumaroyl-CoA ligase (4CL), a heterologous nucleic acid sequence coding for a coumaroyl-CoA 3-hydroxylase (CCoA3H) and a heterologous nucleic acid sequence coding for an acyl-coenzyme A thioesterase. The invention additionally relates to a recombinant microorganism comprising a heterologous nucleic acid sequence coding for a caffeic acid O-methyltransferase. It also relates to the use of these microorganisms for producing ferulic acid and/or caffeic acid and to methods for producing caffeic acid and/or ferulic acid using said microorganisms.

### FIELD OF THE INVENTION

The present invention relates to the use of a recombinant microorganism for producing an organic acid via the hydroxylation and then hydrolysis of a thioester of coenzyme A, and more particularly to a recombinant microorganism capable of producing phenylpropanoid compounds. It also relates to the production methods using such microorganisms.

### TECHNOLOGICAL BACKGROUND

Ferulic acid is a phenylpropanoid derived from cinnamic acid and endowed with antioxidant and free-radical-scavenging properties. Ferulic acid can be used as a precursor in the manufacture of antimicrobial substances for soaps, fragrances and cosmetics, and also for the production of vanillin and sinapic acid. It also finds applications in the medical field, in particular because of its antioxidant properties.

Ferulic acid is obtained by the biodegradation of lignocellulosic biomass. However, the amount of product obtained by this process is relatively low and consequently leads to a particularly high per-kilogramme production cost for these products.

There is therefore a real need for a biosynthesis process that makes it possible to obtain this type of compounds inexpensively.

### SUMMARY OF THE INVENTION

According to a first aspect, the present invention relates to a recombinant microorganism comprising a heterologous nucleic acid sequence coding for a 4-coumaroyl-CoA ligase (4CL), a heterologous nucleic acid sequence coding for a coumaroyl-CoA 3-hydroxylase (CCoA3H) and a heterologous nucleic acid sequence coding for an acyl-coenzyme A thioesterase.

Preferably, said 4CL comprises a sequence chosen from the sequences SEQ ID NOs: 5 to 9 and the polypeptides comprising a sequence having at least 60, 70, 80, 85, 90 or 95%, preferably at least 80, 85, 90 or 95%, sequence identity with one of these sequences and exhibiting 4CL activity.

Preferably, said CCoA3H comprises a sequence chosen from the sequences SEQ ID NOs: 42 to 67 and the polypeptides comprising a sequence having at least 60, 70, 80, 85, 90 or 95%, preferably at least 80, 85, 90 or 95%, sequence identity with one of these sequences and exhibiting CCoA3H activity.

Preferably, said acyl-coenzyme A thioesterase comprises a sequence chosen from the sequences SEQ ID NOs: 1, 2 and 39 and the polypeptides comprising a sequence having at least 60, 70, 80, 85, 90 or 95%, preferably at least 80, 85, 90 or 95%, sequence identity with one of these sequences and exhibiting acyl-coenzyme A thioesterase activity. The microorganism may additionally comprise a heterologous nucleic acid sequence coding for a caffeoyl-CoA O-methyltransferase (CCoAMT), preferably a CCoAMT comprising a sequence chosen from the sequences SEQ ID NOs: 10 to 16, 40 and 41, and the polypeptides comprising a sequence having at least 60, 70, 80, 85, 90 or 95%, preferably at least 80, 85, 90 or 95%, sequence identity with one of these sequences and exhibiting CCoAMT activity.

In the presence of a CCoAMT, the microorganism may additionally comprise a heterologous nucleic acid sequence coding for a CCR and a heterologous nucleic acid sequence coding for an ALDH, preferably said CCR comprising a sequence chosen from the sequence SEQ ID NO: 4 and the polypeptides comprising a sequence having at least 60, 70, 80, 85, 90 or 95%, preferably at least 80, 85, 90 or 95%, sequence identity with the sequence SEQ ID NO: 4 and exhibiting CCR activity; and/or said ALDH comprises a sequence chosen from the sequence SEQ ID NO: 3 and the polypeptides comprising a sequence having at least 60, 70, 80, 85, 90 or 95%, preferably at least 80, 85, 90 or 95%, sequence identity with the sequence SEQ ID NO: 3 and exhibiting ALDH activity.

Alternatively or in addition to a sequence coding for CCoAMT, the microorganism may additionally comprise a heterologous nucleic acid sequence coding for caffeic acid O-methyltransferase (COMT), a COMT comprising a sequence chosen from the sequences SEQ ID NOs: 72 to 92, and the polypeptides comprising a sequence having at least 60, 70, 80, 85, 90 or 95%, preferably at least 80, 85, 90 or 95%, sequence identity with one of these sequences and exhibiting COMT activity, and more particularly preferably a COMT comprising a sequence chosen from the sequences SEQ ID NOs: 71 to 81, and the polypeptides comprising a sequence having at least 60, 70, 80, 85, 90 or 95%, preferably at least 80, 85, 90 or 95%, sequence identity with one of these sequences and exhibiting COMT activity.

Preferably, the microorganism is a bacterium, a yeast or a fungus. In particular, the microorganism may be a bacterium, preferably E. coli. Preferably, the microorganism is a yeast, in particular a yeast of the genus Saccharomyces.

The recombinant microorganism according to the invention may additionally comprise
a heterologous nucleic acid sequence coding for a tyrosine ammonia lyase (TAL), preferably comprising a sequence chosen from SEQ ID NOs: 19, 30 and 68 and the polypeptides comprising a sequence having at least 60, 70, 80, 85, 90 or 95%, preferably at least 80, 85, 90 or 95%, sequence identity with the sequence SEQ ID NO: 19, 30 or 68 and exhibiting a tyrosine ammonia lyase activity, and more particularly preferably a sequence chosen from SEQ ID NO: 19 and the polypeptides comprising a sequence having at least 60, 70, 80, 85, 90 or 95%, preferably at least 80, 85, 90 or 95%, sequence identity with the sequence SEQ ID NO: 19 and exhibiting a tyrosine ammonia lyase activity; and/or
a heterologous nucleic acid sequence coding for a phenylalanine ammonia lyase (PAL) and a heterologous nucleic acid sequence coding for a cinnamate 4-hydroxylase (C4H), preferably a phenylalanine ammonia lyase comprising a sequence chosen from SEQ ID NOs: 20, 69, 31 and 32 and the polypeptides comprising a sequence having at least 60, 70, 80, 85, 90 or 95%, preferably at least 80, 85, 90 or 95%, sequence identity with the sequence SEQ ID NO: 20, 69, 31 or 32 and exhibiting a phenylalanine ammonia lyase activity, in particular a phenylalanine ammonia lyase comprising a sequence chosen from SEQ ID NO: 20 and the polypeptides comprising a sequence having at least 60, 70, 80, 85, 90 or 95%, preferably at least 80, 85, 90 or 95%, sequence identity with the sequence SEQ ID NO: 20 and exhibiting a phenylalanine ammonia lyase activity, and a cinnamate 4-hydroxylase comprising a sequence chosen from SEQ ID NOs: 21, 33, 34 and 70 and the polypeptides comprising a sequence having at least 60, 70, 80, 85, 90 or 95%, preferably at least 80, 85, 90 or 95%, sequence identity with the sequence SEQ ID NO: 21, 33, 34 or 70 and exhibiting a cinnamate 4-hydroxylase activity, in particular a cinnamate 4-hydroxylase comprising a sequence chosen from SEQ ID NO: 21 and the polypeptides comprising a sequence having at least 60, 70, 80, 85, 90 or 95%, preferably at least 80, 85, 90 or 95%, sequence identity with the sequence SEQ ID NO: 21 and exhibiting a cinnamate 4-hydroxylase activity.

The recombinant microorganism according to the invention may additionally comprise a heterologous nucleic acid sequence coding for a phospho-2-dehydro-3-deoxyheptonate aldolase that is resistant to feedback by tyrosine and/or a heterologous nucleic acid sequence coding for a chorismate mutase that is resistant to feedback by tyrosine.

Preferably, in the recombinant microorganism according to the invention, a gene coding for a phenylpyruvate decarboxylase is inactivated and/or a gene coding for a ferulic acid decarboxylase is inactivated.

In a second aspect, the present invention relates to a method for producing a phenylpropanoid chosen from caffeic acid and ferulic acid, preferably ferulic acid, comprising culturing a recombinant microorganism according to the invention and optionally harvesting and/or purifying said phenylpropanoid.

Preferably, the phenylpropanoid is ferulic acid and the recombinant microorganism is a microorganism according to the invention comprising (i) a heterologous nucleic acid sequence coding for a 4-coumaroyl-CoA ligase (4CL), a heterologous nucleic acid sequence coding for a coumaroyl-CoA 3-hydroxylase (CCoA3H), a heterologous nucleic acid sequence coding for an acyl-coenzyme A thioesterase and (ii) a heterologous nucleic acid sequence coding for a caffeoyl-CoA O-methyltransferase (CCoAMT) and/or a heterologous nucleic acid sequence coding for caffeic acid O-methyltransferase (COMT).

In another aspect, the present invention relates to the use of a recombinant microorganism according to the invention for producing a phenylpropanoid chosen from ferulic acid and caffeic acid, preferably ferulic acid.

Preferably, the recombinant microorganism is a microorganism according to the invention which comprises (i) a heterologous nucleic acid sequence coding for a 4-coumaroyl-CoA ligase (4CL), a heterologous nucleic acid sequence coding for a coumaroyl-CoA 3-hydroxylase (CCoA3H), a heterologous nucleic acid sequence coding for an acyl-coenzyme A thioesterase and (ii) a heterologous nucleic acid sequence coding for a caffeoyl-CoA O-methyltransferase (CCoAMT) and/or a heterologous nucleic acid sequence coding for caffeic acid O-methyltransferase (COMT), and the phenylpropanoid is ferulic acid.

In another aspect, the present invention relates to a recombinant microorganism comprising a heterologous nucleic acid sequence coding for a caffeic acid O-methyltransferase (COMT), said COMT comprising a sequence chosen from the sequences SEQ ID NOs: 73 to 92, and the polypeptides comprising a sequence having at least 60, 70, 80, 85, 90 or 95%, preferably at least 80, 85, 90 or 95%, sequence identity with one of these sequences and exhibiting COMT activity, preferably a sequence chosen from the sequences SEQ ID NOs: 74, 76, 79, 80 and 82, and the polypeptides comprising a sequence having at least 80, 85, 90 or 95% sequence identity with one of these sequences and exhibiting COMT activity.

The microorganism comprising a heterologous nucleic acid sequence coding for a COMT may additionally comprise a heterologous nucleic acid sequence coding for a 4-hydroxyphenylacetate 3-monooxygenase oxygenase (HpaB), a heterologous nucleic acid sequence coding for a 4-hydroxyphenylacetate 3-monooxygenase reductase (HpaC), a heterologous nucleic acid sequence coding for a CPR-dependent p-coumarate 3-hydroxylase (C3H), a heterologous nucleic acid sequence coding for a cytochrome P450 reductase (CPR), a heterologous nucleic acid sequence coding for a tyrosine ammonia lyase (TAL), a heterologous nucleic acid sequence coding for a phenylalanine ammonia lyase (PAL) and/or a heterologous nucleic acid sequence coding for a cinnamate 4-hydroxylase (C4H), preferably a heterologous nucleic acid sequence coding for a 4-hydroxyphenylacetate 3-monooxygenase oxygenase (HpaB), a heterologous nucleic acid sequence coding for a 4-hydroxyphenylacetate 3-monooxygenase reductase (HpaC), a heterologous nucleic acid sequence coding for a cytochrome P450 reductase (CPR), a heterologous nucleic acid sequence coding for a tyrosine ammonia lyase (TAL), a heterologous nucleic acid sequence coding for a phenylalanine ammonia lyase (PAL) a heterologous nucleic acid sequence coding for a cinnamate 4-hydroxylase (C4H).

A gene coding for a phenylpyruvate decarboxylase and/or a gene coding for a ferulic acid decarboxylase may be inactivated.

The microorganism may be a bacterium, a yeast or a fungus. In particular, the microorganism may be a bacterium, preferably E. coli. Preferably, the microorganism is a yeast, in particular a yeast of the genus Saccharomyces.

The present invention also relates to a method for producing ferulic acid, comprising culturing a recombinant microorganism according to the invention comprising a heterologous nucleic acid sequence coding for a COMT, and optionally harvesting and/or purifying the ferulic acid produced. It also relates to the use of said recombinant microorganism for producing ferulic acid.

### DESCRIPTION OF THE FIGURES

FIG. 1: Production of ferulic acid from caffeic acid of yeasts expressing the thioesterase of SEQ ID NO: 1 (Thio1), the thioesterase of SEQ ID NO: 2 (Thio3), or the cinnamoyl-CoA reductase of SEQ ID NO: 4 and the aldehyde dehydrogenase of SEQ ID NO: 3 (CCR ALDH). The negative control (ø) does not express any of these enzymes.

FIG. 2: Ferulic acid production of strains 339 to 343, 345, 347, 367 to 371, 373, 375, 423 to 427, 429, 431, 451 to 455, 457 and 459 (CCoAMT1: SEQ ID NO: 10, CCoAMT2: SEQ ID NO: 11, CCoAMT3: SEQ ID NO: 12, CCoAMT4: SEQ ID NO: 13, CCoAMT5: SEQ ID NO: 14, CCoAMT6: SEQ ID NO: 15, CCoAMT7: SEQ ID NO: 16, 4CL1: SEQ ID NO: 5, 4CL5: SEQ ID NO: 6, 4CLA: SEQ ID NO: 8, 4CLB: SEQ ID NO: 9, Thio3: SEQ ID NO: 2) and of negative controls (0: strains 334, 335, 337 and 338), cultured in the presence of caffeic acid.

FIG. 3: Ferulic acid production of strains 595, 596 and 597 cultured in the presence of p-coumaric acid and of the negative control (0: strain 507).

FIG. 4: Ferulic acid production of strains 592, 593 and 594 cultured in the presence of glucose and of the negative control (0: strain 516).

FIG. 5: A pathway for biosynthesis of ferulic acid from glucose.

FIG. 6: Illustration of the enzymatic activities of acyl-coenzyme A thioesterase or of cinnamoyl-CoA reductase and aldehyde deshydrogenase on feruloyl-CoA.

FIG. 7: Production of caffeic acid from glucose proceeding via the CoA pathway, test of different coumaroyl-CoA 3-hydroxylases (CCoA3H). The black line represents the limit of quantification for caffeic acid (LLOQ).

FIG. 8: Production of caffeic acid from glucose proceeding via the CoA pathway, test of CCoA3H14. The black line represents the limit of quantification for caffeic acid (LLOQ).

FIG. 9: Production of caffeic acid from glucose proceeding via the CoA pathway, test of different coumaroyl-CoA 3-hydroxylases (CCoA3H). The black line represents the limit of quantification for caffeic acid (LLOQ).

FIG. 10: Production of caffeic acid from glucose proceeding via the organic acid pathway, test of different coumaroyl-CoA 3-hydroxylases (CCoA3H). The black line represents the limit of quantification for caffeic acid (LLOQ).

FIG. 11: Production of caffeic acid from glucose proceeding via the organic acid pathway, test of different coumaroyl-CoA 3-hydroxylases (CCoA3H). The black line represents the limit of quantification for caffeic acid (LLOQ).

FIG. 12: Production of ferulic acid from glucose of strains VAN1622, VAN1624, YSP226 and YSP228.

FIG. 13: Amounts of ferulic acid produced after 72 h of culture by strains expressing different COMTs (expressed in % relative to the amount produced by the strain expressing the COMT of SEQ ID NO: 72) and amounts of caffeic acid remaining after 72 h of culture (expressed in % relative to the amount of caffeic acid produced by the strain CRTL not expressing COMT).

### DETAILED DESCRIPTION OF THE INVENTION

Synthetic pathways involving compounds associated with coenzyme A (CoA) are hardly used in biotechnology. This is because the difficulty of detecting and measuring the amounts of the synthesis intermediates as well as the impossibility of accumulating them within the cell (operation in flow mode only) make handling thereof delicate. However, the inventors have explored these biosynthetic pathways and have developed a microorganism capable of producing organic acids via the hydrolysis of thioesters of CoA such as ferulic acid or caffeic acid. Besides reduced costs compared to current production techniques by extraction from plant biomass such as rice bran, the production method using this microorganism also has the advantage of proceeding via intermediates associated with CoA and thus incapable of leaving the cells before being converted into the target molecule. This therefore makes it possible to reduce the amount of intermediate compounds that are accumulated in the medium or are degraded, and thus ultimately to have a purer product. The production method according to the invention thus makes it possible to obtain biobased phenylpropanoids via a biosynthetic process that is more environmentally friendly than extraction by hydrolysis from natural biomass such as rice bran, which generates alkaline waste that is difficult to degrade.

The inventors have also identified COMT (caffeic acid O-methyltransferase) enzymes that are particularly efficient for producing ferulic acid from caffeic acid. These enzymes thus enable a significant improvement in the production of ferulic acid obtained from recombinant microorganisms.

### Definitions

As used herein, the term “microorganism” refers to a prokaryotic or eukaryotic microorganism, in particular a yeast, a fungus or a bacterium.

The term “recombinant microorganism” is understood to mean a microorganism which is not found in nature and which contains a genome modified following an insertion, modification or deletion of one or more heterologous genetic elements.

The term “recombinant nucleic acid” is understood to mean a nucleic acid which has been modified and does not exist in nature. For example, this term may denote a coding sequence or a gene which is operatively linked to a promoter which is not the natural promoter. This may also denote a coding sequence in which the introns have been deleted for genes comprising exons and introns.

The term “heterologous” is understood to mean a nucleic acid sequence or a protein which is not naturally present in the host cell and which has been introduced by genetic engineering. The heterologous sequence may be present in the cell in episomal or chromosomal form. The origin of the heterologous sequence may be different from the cell into which it is introduced. However, it may also originate from the same species as the cell into which it is introduced but be considered as heterologous on account of its unnatural environment. For example, the nucleic sequence is heterologous when it is under the control of a promoter other than its natural promoter, or when it is introduced into a location different from that in which it is naturally located. The host cell may contain an endogenous copy of the nucleic sequence prior to the introduction of the heterologous nucleic sequence or it may not contain an endogenous copy. Moreover, the nucleic acid sequence may be heterologous in the sense that the coding sequence has been optimized for expression in the host cell, for example by optimization of codon usage. Preferably, in the present document, the term “heterologous nucleic acid sequence” refers to a nucleic acid sequence which codes for a protein which is heterologous to the host cell, i.e. which is not naturally present in the host cell.

As used herein, the term “endogenous”, relative to the host cell, refers to a genetic element or to a protein that is naturally present in said cell.

The term “gene” or “coding sequence” denotes any nucleic acid coding for a protein. The term “gene” encompasses DNA, such as cDNA (complementary DNA) or gDNA (genomic DNA), and also RNA. The gene may first be prepared via recombinant, enzymatic and/or chemical techniques, and subsequently replicated in a host cell or a system in vitro. The gene typically comprises an open reading frame coding for a desired protein. The gene may contain additional sequences such as a transcription terminator or a signal peptide. As a result of degeneracy of the genetic code, several nucleic acids may code for a particular polypeptide. Thus, the codons in the coding sequence for a given polypeptide may be modified such that optimal expression in a particular cell is obtained, for example by using suitable codon translation tables for this cell. The nucleic acids may also be optimized according to a preferable GC content for said cell and/or to reduce the number of repeat sequences. In certain embodiments, the heterologous nucleic acids were codon-optimized for expression in the host cell concerned. Codon optimization may be performed via routine processes known in the art (see, for example, Welch, M., et al. (2011), Methods in Enzymology 498: 43-66).

The term “operatively linked” denotes a configuration in which a control sequence is placed in a suitable position relative to a coding sequence, such that the control sequence directs the expression of the coding sequence.

The term “control sequences” denotes the nucleic acid sequences required for the expression of a gene. The control sequences may be endogenous or heterologous. Control sequences that are well known and currently used by those skilled in the art will be preferred. Such control sequences comprise, but without being limited thereto, a leader, a polyadenylation sequence, a propeptide sequence, a promoter, a signal peptide sequence and a transcription terminator. Preferably, the control sequences comprise a promoter and a transcription terminator.

The term “expression cassette” denotes a nucleic acid construct comprising a coding region, i.e. a gene, and a regulating region, i.e. a region comprising one or more control sequences, which are operatively linked. Preferably, the control sequences are adapted to the host cell. As used herein, the term “expression vector” denotes a DNA or RNA molecule which comprises an expression cassette. Preferably, the expression vector is a linear or circular, preferably linear, double-stranded DNA molecule. The vector may also comprise an origin of replication, a selection marker, etc.

For the purposes of the present invention, the term “percentage identity” between two nucleic acid sequences or amino acid sequences is understood to denote a percentage of nucleotides or of amino acid residues that are identical between the two sequences to be compared, obtained after the best alignment, this percentage being purely statistical and the differences between the two sequences being distributed randomly and over their entire length. The best alignment or optimal alignment is the alignment for which the percentage identity between the two sequences to be compared, as calculated below, is the highest. Sequence comparisons between two nucleic acid or amino acid sequences are conventionally performed by comparing these sequences after they have been optimally aligned, said comparison being performed by segment or by comparison window to identify and compare the local regions with sequence similarity. The alignment for the purposes of determining the percentage of amino acid sequence identity may be performed in various ways that are well known in the field, for example by using computer software available on the Internet, such as http://www.clustal.org/omega/or http://www.ebi.ac.uk/Tools/emboss/. A person skilled in the art can determine the appropriate parameters for measuring the alignment, including any algorithm necessary to obtain a maximum alignment over the entire length of the sequences compared. For the purposes of the present invention, the values of the percentage of amino acid sequence identity refer to values generated using the EMBOSS Needle pairwise sequence alignment program which creates an optimal global alignment of two sequences by means of the Needleman-Wunsch algorithm, in which all the search parameters are defined by default Notation matrix=BLOSUM62, Gap open=10, Gap extension=0.5, end gap penalty=false, open end gap=10 and extended end gap=0.5. In certain embodiments, all the percentages of identity mentioned in the present patent application may be set at least 60%, at least 70%, at least 80%, at least 85%, preferably at least 90% identity, more preferably at least 95% identity. Alternatively, the percentages of sequence identity mentioned in the present patent application may be set at least 96%, at least 97%, at least 98% or at least 99% sequence identity. In particular, the embodiments in which all the percentages of sequence identity of the enzymes are at least 80% or at least 85%, preferably at least 90% or at least 95% sequence identity are considered as described. The embodiments in which all the percentages of sequence identity of the enzymes are at least 96%, at least 97%, at least 98% or at least 99% sequence identity are also considered as described. In one embodiment, the polypeptides may contain 1, 2, 3, 4, 5, 6, 7, 8, 9 or 10 additions, substitutions or deletions relative to the sequences described in the SEQ ID NOs. In particular, these additions, substitutions or deletions may be introduced at the N-terminal end, the C-terminal end or at both ends. The polypeptides may optionally be in the form of a fusion protein. In this case, the percentage identity is calculated only on the domain of the fusion protein exhibiting the desired activity.

The terms “overexpression” and “increased expression” as used herein are used interchangeably and mean that the expression of a coding sequence or of a protein is increased relative to the unmodified host cell, for example a wild-type cell or a cell not comprising the genetic modifications described herein. The term “wild-type” is understood to mean an unmodified cell existing in nature. The increased expression of a protein is usually obtained by increasing the expression of the gene coding for said protein. In embodiments in which the gene or the protein is not naturally present in the microorganism of the invention, i.e. a heterologous gene or protein, the terms “overexpression” and “expression” may be used interchangeably. To increase the expression of a gene, a person skilled in the art can use any known technique such as increasing the number of copies of the gene in the cell, using a promoter inducing a high level of expression of the gene, i.e. a strong promoter, using elements which stabilize the corresponding messenger RNA or particular RBS (ribosome binding site) sequences. In particular, overexpression may be obtained by increasing the number of copies of the gene in the cell. One or more copies of the gene may be introduced into the genome via recombination processes, known to those skilled in the art, including gene replacement or multi-copy insertion. Preferably, an expression cassette comprising the gene is integrated into the genome. As a variant, the gene may be carried by an expression vector, preferably a plasmid, comprising an expression cassette with the gene of interest preferably placed under the control of a suitable promoter. The expression vector may be present in the host cell in one or more copies, depending on the nature of the origin of replication. Overexpression of a gene may also be obtained by using a promoter which induces a high level of expression of the gene. For example, the promoter of an endogenous gene may be replaced with a stronger promoter, i.e. a promoter which induces a higher level of expression. The endogenous gene under the control of a promoter which is not the natural promoter is termed a heterologous nucleic acid. The promoters that are suitable for use in the present invention are known to those skilled in the art and may be constitutive or inducible, and may be endogenous or heterologous.

The term “comprising” also denotes “consisting of” or “consisting essentially of”. Thus, the embodiments in which the term “comprising” is replaced by the term “consisting of” or “consisting essentially of” are also described in this document. The term “consisting essentially of” is understood to mean that the sequence may contain 1, 2, 3, 4, 5, 6, 7, 8, 9 or 10 additions, substitutions or deletions relative to the sequences described in the SEQ ID NOs.

### Microorganisms

The microorganism according to the present invention may be a eukaryotic or prokaryotic microorganism.

According to a first embodiment, the microorganism is a eukaryotic microorganism, preferably chosen from yeasts and fungi.

According to a preferred embodiment, the microorganism is a yeast, in particular a yeast from the order Saccharomycetales, Sporidiobolales or Schizosaccharomycetales. The yeast may for example be selected from the yeasts of the genus Saccharomyces, Pichia, Kluyveromyces, Schizosaccharomyces, Candida, Lipomyces, Rhodotorula, Rhodosporidium, Yarrowia, Debaryomyces, Komagataella, Scheffersomyces, Torulaspora or Zygosaccharomyces. In particular, the yeast may be chosen from the species Saccharomyces cerevisiae, Saccharomyces carlsbergensis, Saccharomyces diastaticus, Saccharomyces douglasii, Saccharomyces kluyveri, Saccharomyces norbensis, Saccharomyces oviformis, Pichia pastoris, Kluyveromyces lactis, Kluyveromyces marxianus, Schizosaccharomyces pombe, Candida albicans, Candida tropicalis, Rhodotorula glutinis, Rhodosporidium toruloides, Yarrowia lipolytica, Debaryomyces hansenii and Lipomyces starkeyi.

According to a particularly preferred embodiment, the microorganism is a yeast belonging to the genus Saccharomyces, preferably a yeast chosen from the species Saccharomyces cerevisiae, Saccharomyces carlsbergensis, Saccharomyces diastaticus, Saccharomyces douglasii, Saccharomyces kluyveri, Saccharomyces norbensis and Saccharomyces oviformis. Most particularly preferably, the microorganism is a yeast of the species Saccharomyces cerevisiae.

Alternatively, the microorganism may be a fungus, preferably a filamentous fungus, in particular a fungus chosen from the fungi of the genera Aspergillus, Trichoderma, Neurospora, Podospora, Endothia, Mucor, Cochiobolus and Pyricularia. More particularly preferably, the fungus is chosen from Aspergillus nidulans, Aspergillus niger, Aspergillus awomari, Aspergillus oryzae, Aspergillus terreus, Neurospora crassa, Trichoderma reesei and Trichoderma viride.

According to a second embodiment, the microorganism is a prokaryotic microorganism, preferably a bacterium. In particular, the microorganism may be a bacterium chosen from the bacterium of the phylum Acidobacteria, Actinobacteria, Aquificae, Bacterioidetes, Chlamydiae, Chlorobi, Chloroflexi, Chrysiogenetes, Cyanobacteria, Deferribacteres, Deinococcus-Thermus, Dictyoglomi, Fibrobacteres, Firmicutes, Fusobacteria, Gemmatimonadetes, Nitrospirae, Planctomycetes, Proteobacteria, Spirochaetes, Thermodesulfobacteria, Thermomicrobia, Thermotogae, or Verrucomicrobia. Preferably, the bacterium belongs to the genus Acaryochloris, Acetobacter, Actinobacillus, Agrobacterium, Alicyclobacillus, Anabaena, Anacystis, Anaerobiospirillum, Aquifex, Arthrobacter, Arthrospira, Azobacter, Bacillus, Brevibacterium, Burkholderia, Chlorobium, Chromatium, Chlorobaculum, Clostridium, Corynebacterium, Cupriavidus, Cyanothece, Enterobacter, Deinococcus, Erwinia, Escherichia, Geobacter, Gloeobacter, Gluconobacter, Hydrogenobacter, Klebsiella, Lactobacillus, Lactococcus, Mannheimia, Mesorhizobium, Methylobacterium, Microbacterium, Microcystis, Nitrobacter, Nitrosomonas, Nitrospina, Nitrospira, Nostoc, Phormidium, Prochlorococcus, Pseudomonas, Ralstonia, Rhizobium, Rhodobacter, Rhodococcus, Rhodopseudomonas, Rhodospirillum, Salmonella, Scenedesmun, Serratia, Shigella, Staphylococcus, Streptomyces, Synechoccus, Synechocystis, Thermosynechococcus, Trichodesmium or Zymomonas. More preferably still, the bacterium is chosen from the species Agrobacterium tumefaciens, Anaerobiospirillum succiniciproducens, Actinobacillus succinogenes, Aquifex aeolicus, Aquifex pyrophilus, Bacillus subtilis, Bacillus amyloliquefaciens, Brevibacterium ammoniagenes, Brevibacterium immariophilum, Clostridium pasteurianum, Clostridium ljungdahlii, Clostridium acetobutylicum, Clostridium beigerinckii, Corynebacterium glutamicum, Cupriavidus necator, Cupriavidus metallidurans, Enterobacter sakazakii, Escherichia coli, Gluconobacter oxydans, Hydrogenobacter thermophilus, Klebsiella oxytoca, Lactococcus lactis, Lactobacillus plantarum, Mannheimia succiniciproducens, Mesorhizobium loti, Pseudomonas aeruginosa, Pseudomonas mevalonii, Pseudomonas pudica, Pseudomonas putida, Pseudomonas fluorescens, Rhizobium etli, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, Staphylococcus aureus, Streptomyces coelicolor, Zymomonas mobilis, Acaryochloris marina, Anabaena variabilis, Arthrospira platensis, Arthrospira maxima, Chlorobium tepidum, Chlorobaculum sp., Cyanothece sp., Gloeobacter violaceus, Microcystis aeruginosa, Nostoc punctiforme, Prochlorococcus marinus, Synechococcus elongatus, Synechocystis sp., Thermosynechococcus elongatus, Trichodesmium erythraeum and Rhodopseudomonas palustris. According to a particular embodiment, the microorganism is an Escherichia coli bacterium, for example chosen from E. coli BL21, E. coli BL21 (DE3), E. coli MG1655, E. coli W31 10 and derivatives thereof. According to a particular alternative embodiment, the microorganism is a bacterium of the genus Streptomyces, in particular Streptomyces venezuelae.

According to a preferred embodiment, the microorganism is a yeast, a bacterium or a fungus, preferably a yeast or a bacterium. In particular, the microorganism may be chosen from an Escherichia coli bacterium and a Saccharomyces cerevisiae yeast.

According to an embodiment preferred above all, the microorganism is a yeast, preferably a yeast belonging to the genus Saccharomyces, in particular a Saccharomyces cerevisiae yeast.

Production of Caffeic Acid from p-Coumaric Acid

By virtue of the thiol function of the cysteamine, coenzyme A is capable of forming, with the carboxyl functions of certain compounds such as caffeic acid or p-coumaric acid, thioesters referred to as carboxyl-CoA.

As used herein, the term “carboxyl-CoA” refers to a thioester of coenzyme A in which hydrolysis of the thioester bond generates a carboxyl group. Examples of carboxyl-CoA are p-coumaroyl-CoA, caffeoyl-CoA and feruloyl-CoA.

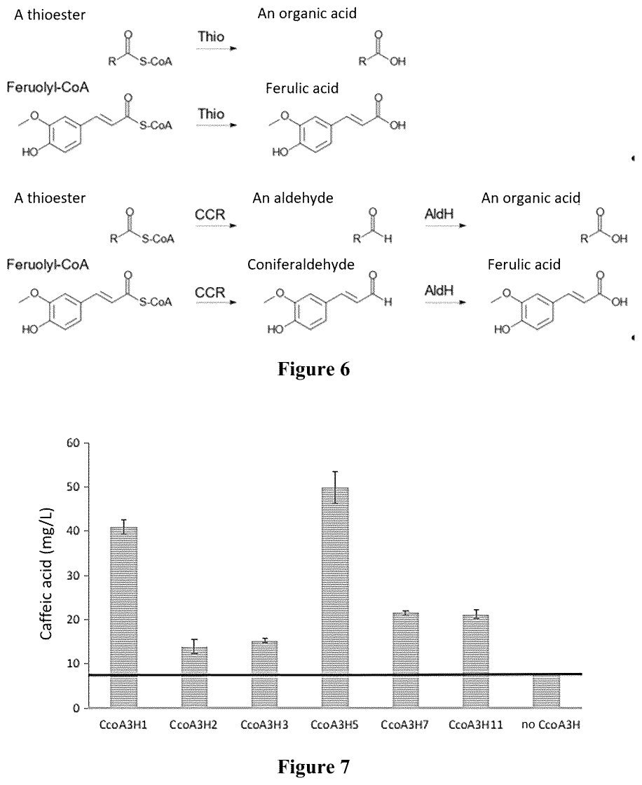

The recombinant microorganism according to the present invention is genetically modified to produce phenylpropanoids, namely ferulic acid and/or caffeic acid, from p-coumaric acid, proceeding via intermediate compounds which are carboxyl-CoAs, namely p-coumaroyl-CoA, caffeoyl-CoA and feruloyl-CoA. (cf. FIGS. 5 and 6). In particular, this microorganism is capable (i) of producing ferulic acid from feruloyl-CoA, caffeoyl-CoA, caffeic acid, p-coumaric acid or p-coumaroyl-CoA and/or (ii) of producing caffeic acid from caffeoyl-CoA, p-coumaric acid or p-coumaroyl-CoA.

In plants, the lignin synthesis pathway involves compounds coupled to CoA which are then hydrolyzed. This type of hydrolysis is carried out in 2 steps. The first step consists in liberating the substrate in its aldehyde form. The second consists in oxidizing the aldehyde compound to its acid form (Fraser and Chapple, Arabidopsis Book. 2011; 9:e0152). Certain plants such as Petunia hybrida or Curcuma longa L. appear to also be capable of directly hydrolyzing the phenylpropanoyl-CoAs via thioesterases which catalyze the hydrolysis of a thioester bond while liberating a carboxylic acid and a thiol group (Adebesin et al., Planti. 2018 January; 93: 905-916; Ramirez-Ahumada et al., Phytochemistry. 2006 September; 67(18): 2017-29) and which are especially described in the pathway for the biosynthesis of fatty acids. In addition, this pathway for synthesizing lignin also involves other enzymes such as 4-coumaroyl-CoA ligase or coumaroyl-CoA 3-hydroxylase that are involved in particular in the production of the hydroxyphenyl and guaiacyl units of lignin (Zhong et al. Plant Physiology, Volume 124, Issue 2, October 2000, Pages 563-578).

The inventors have demonstrated herein, surprisingly, that these mechanisms could be used in a microorganism, and in particular in a yeast, to produce phenylpropanoids such as ferulic acid and/or caffeic acid.

The recombinant microorganism according to the invention comprises a heterologous nucleic acid sequence coding for a 4-coumaroyl-CoA ligase (4CL), a heterologous nucleic acid sequence coding for a coumaroyl-CoA 3-hydroxylase (CCoA3H) and a heterologous nucleic acid sequence coding for an acyl-coenzyme A thioesterase.

As used herein, the term “4-coumaryl-CoA ligase” or “4CL” refers to an enzyme capable of producing caffeoyl-CoA from caffeic acid and CoA and/or p-coumaroyl-CoA from p-coumaric acid and CoA. This enzyme belongs to the class EC 6.2.1.12.

The detection of this activity may be achieved by any method known to those skilled in the art, in vivo or in vitro. In particular, to determine whether there is any 4-coumarate CoA ligase activity, an enzymatic test may be effected consisting of the in vitro incubation of a mixture composed of the enzyme to be tested (putative 4-coumarate CoA ligase), caffeic acid or p-coumaric acid, ATP and CoA under optimal conditions (pH, temperature, ions, etc.). After a certain incubation time, the appearance of caffeoyl-CoA (in the presence of caffeic acid) or of p-coumaroyl-CoA (in the presence of p-coumaric acid) is observed in UV spectrophotometry with a given wavelength.

The 4CL may be a plant enzyme, preferably of a plant of the genus Abies, Arabidopsis, Agastache, Amorpha, Brassica, Citrus, Cathaya, Cedrus, Crocus, Larix, Festuca, Glycine, Juglans, Keteleeria, Lithospermum, Lolium, Lotus, Lycopersicon, Malus, Medicago, Mesembryanthemum, Nicotiana, Nothotsuga, Oryza, Phaseolus, Pelargonium, Petroselinum, Physcomitrella, Picea, Prunus, Pseudolarix, Pseudotsuga, Rosa, Rubus, Ryza, Saccharum, Suaeda, Pinus, Populus, Solanum, Thellungiella, Triticum, Tsuga, Vitis or Zea. Alternatively, this enzyme may be an enzyme produced by a microorganism, for example of the genus Aspergillus, Mycosphaerella, Mycobacterium, Neisseria, Neurospora, Streptomyces, Rhodobacter or Yarrowia.

Preferably, the 4CL is a plant enzyme, in particular an enzyme of a plant of the genus Arabidopsis. Citrus or Populus. More specifically, the 4CL may be a 4CL from Arabidopsis thaliana, in particular a 4CL described in one of the sequences SEQ ID NOs: 5, 7 and 9, a 4CL from Citrus clementina, in particular a 4CL described in SEQ ID NO: 6 or a 4CL from Populus tomentosa, in particular a 4CL described in SEQ ID NO: 8.

According to one embodiment, the 4CL comprises a sequence chosen from the sequences SEQ ID NOs: 5 to 9 and the polypeptides comprising a sequence having at least 60, 70, 80, 85, 90 or 95% sequence identity with one of these sequences and exhibiting 4CL activity. According to a preferred embodiment, the 4CL comprises a sequence chosen from the sequences SEQ ID NOs: 5, 6, 8 and 9 and the polypeptides comprising a sequence having at least 60, 70, 80, 85, 90 or 95% sequence identity with one of these sequences and exhibiting 4CL activity.

According to a very particularly preferred embodiment, the 4CL comprises a sequence chosen from the sequences SEQ ID NOs: 6 and 8 and the polypeptides comprising a sequence having at least 60, 70, 80, 85, 90 or 95% sequence identity with one of these sequences and exhibiting 4CL activity.

As used herein, the term “coumaroyl-CoA 3-hydroxylase” or “p-coumaroyl-CoA 3-hydroxylase” or “CCoA3H” refers to an enzyme capable of producing caffeoyl-CoA from p-coumaroyl-CoA. This enzyme belongs to the class EC 1.14.13.x. The detection of this activity may be achieved by any method known to those skilled in the art, in vivo or in vitro. In particular, to determine whether there is any CCoA3H activity, an enzymatic test may be effected consisting of the in vitro incubation of a mixture composed of the enzyme to be tested (putative CCoA3H), p-coumaric acid, a 4CL enzyme, ATP and CoA under optimal conditions (pH, temperature, ions, etc.). After a certain incubation time, the appearance of caffeoyl-CoA is observed in UV spectrophotometry with a given wavelength. The CCoA3H activity may also be detected in vivo, in particular using the method described in the experimental part hereinafter. In particular, the CCoA3H activity may be detected in vivo using a microorganism expressing the enzyme to be tested. The substrate of said enzyme (p-coumaroyl-CoA) is synthesized by the microorganism on the basis of p-coumaric acid and a 4CL enzyme. The caffeoyl-CoA produced by an enzyme exhibiting CCoA3H activity may then be detected by any method, preferably by an HPLC technique. Particularly preferably, the CCoA3H activity may be detected in vivo using a microorganism expressing the enzyme to be tested and capable of converting caffeoyl-CoA to caffeic acid and not possessing any other pathway enabling the production of caffeic acid. The CCoA3H activity is detected via the production of caffeic acid in the presence of p-coumaric acid, preferably by a UPLC (ultra performance liquid chromatography) technique coupled with a high-resolution mass spectrometer.

Preferably, the CCoA3H is a plant enzyme, in particular an enzyme of a plant of the genus Vigna, Glycine, Jatropha, Acacia, Populus, Triticum, Salvia, Cosmos, Trifolium, Lonicera, Nyssa, Pyrus, Eucalyptus, Gossypium, Zostera, Aquilegia, Actinidia, Medicago, Malus, Ricinus, Sorghum, Nicotiana, Raphanus or Ipomoea.

More specifically, the CCoA3H may be a CCoA3H from Vigna angularis, in particular a CCoA3H described in the sequence SEQ ID NO: 42, a CCoA3H from Glycine max, in particular a CCoA3H described in the sequence SEQ ID NO: 43, a CCoA3H from Jatropha curcas, in particular a CCoA3H described in the sequence SEQ ID NO: 44, a CCoA3H from Acacia koa, in particular a CCoA3H described in the sequence SEQ ID NO: 45, a CCoA3H from Populus tomentosa, in particular a CCoA3H described in the sequence SEQ ID NO: 46, a CCoA3H from Populus alba x Populus grandidentata, in particular a CCoA3H described in the sequence SEQ ID NO: 47, a CCoA3H from Triticum turgidum subsp. durum, in particular a CCoA3H described in the sequence SEQ ID NO: 48, a CCoA3H from Salvia miltiorrhiza, in particular a CCoA3H described in the sequence SEQ ID NO: 49, a CCoA3H from Cosmos sulphureus, in particular a CCoA3H described in the sequence SEQ ID NO: 50, a CCoA3H from Trifolium pratense, in particular a CCoA3H described in the sequence SEQ ID NO: 51, a CCoA3H from Lonicerajaponica, in particular a CCoA3H described in the sequence SEQ ID NO: 52, a CCoA3H from Nyssa sinensis, in particular a CCoA3H described in the sequence SEQ ID NO: 53, a CCoA3H from Pyrus ussuriensis x Pyrus communis, in particular a CCoA3H described in the sequence SEQ ID NO: 54, a CCoA3H from Eucalyptus grandis, in particular a CCoA3H described in the sequence SEQ ID NO: 55, a CCoA3H from Gossypium raimondii, in particular a CCoA3H described in the sequence SEQ ID NO: 56, a CCoA3H from Zostera marina, in particular a CCoA3H described in the sequence SEQ ID NO: 57, a CCoA3H from Aquilegia coerulea, in particular a CCoA3H described in the sequence SEQ ID NO: 58, a CCoA3H from Actinidia chinensis var. chinensis, in particular a CCoA3H described in the sequence SEQ ID NO: 59, a CCoA3H from Medicago truncatula, in particular a CCoA3H described in the sequence SEQ ID NO: 60, a CCoA3H from Malus baccata, in particular a CCoA3H described in the sequence SEQ ID NO: 61, a CCoA3H from Ricinus communis, in particular a CCoA3H described in the sequence SEQ ID NO: 62, a CCoA3H from Sorghum bicolor, in particular a CCoA3H described in the sequence SEQ ID NO: 63, a CCoA3H from Populus euphratica, in particular a CCoA3H described in the sequence SEQ ID NO: 64, a CCoA3H from Nicotiana tabacum, in particular a CCoA3H described in the sequence SEQ ID NO: 65, a CCoA3H from Raphanus sativus, in particular a CCoA3H described in the sequence SEQ ID NO: 66, or a CCoA3H from Ipomoea nil, in particular a CCoA3H described in the sequence SEQ ID NO: 67.

According to one embodiment, the CCoA3H comprises a sequence chosen from the sequences SEQ ID NOs: 42 to 67 and the polypeptides comprising a sequence having at least 60, 70, 80, 85, 90 or 95% sequence identity with one of these sequences and exhibiting CCoA3H activity.

According to a particular embodiment, the CCoA3H comprises a sequence chosen from the sequences SEQ ID NOs: 42, 45, 46, 47, 48, 49, 51, 52, 54, 59 and 65 and the polypeptides comprising a sequence having at least 60, 70, 80, 85, 90 or 95% sequence identity with one of these sequences and exhibiting CCoA3H activity.

According to a preferred embodiment, the CCoA3H comprises a sequence chosen from the sequences SEQ ID NOs: 42, 45 and 49 and the polypeptides comprising a sequence having at least 60, 70, 80, 85, 90 or 95% sequence identity with one of these sequences and exhibiting CCoA3H activity.

According to a very particularly preferred embodiment, the CCoA3H comprises a sequence chosen from the sequence SEQ ID NO: 49 and the polypeptides comprising a sequence having at least 60, 70, 80, 85, 90 or 95% sequence identity with said sequence and exhibiting CCoA3H activity.

As used herein, the term “acyl-coenzyme A thioesterase” or “Thio” refers to an enzyme endowed with an acyl-coenzyme A thioesterase activity, that is to say an enzyme capable of hydrolyzing the ester bond of a carboxyl-CoA and thus of liberating the CoA on the one hand and a carboxylic acid on the other (cf. FIG. 6). This term refers in particular to an enzyme capable of hydrolyzing the thioester bond of feruloyl-CoA to thus produce ferulic acid and/or of hydrolyzing the thioester bond of caffeoyl-CoA to thus produce caffeic acid. The detection of this activity may be achieved by any method known to those skilled in the art, in vivo or in vitro. Acyl-coenzyme A thioesterase activity can be detected in vitro, for example as described in the article by Adebesin et a/. (Plant J. 2018 March; 93(5): 905-916). In particular, in order to determine whether there is any acyl-CoA thioesterase activity, an enzymatic test may be carried out consisting of the in vitro incubation of a mixture composed of the enzyme to be tested (putative acyl-CoA thioesterase), and of a carboxyl-CoA substrate under optimal conditions (pH, temperature, ions, etc.). After a certain incubation time, the appearance of the carboxylic acid obtained by hydrolysis of the ester bond of the carboxyl-CoA substrate can be observed either in UV spectrophotometry at a given wavelength or by HPLC. The acyl-coenzyme A thioesterase activity may also be detected in vivo, in particular using the method described in the experimental part hereinafter. In particular, the acyl-coenzyme A thioesterase activity may be detected in vivo using a microorganism expressing the enzyme to be tested. The carboxyl-CoA substrate of said enzyme is either synthesized by the microorganism or supplied to the culture medium. The carboxylic acid produced by an enzyme exhibiting the desired activity may then be detected by any method, preferably by an HPLC technique. In particular, the acyl-coenzyme A thioesterase activity may be detected via the production of ferulic acid in the presence of feruloyl-CoA and/or the production of caffeic acid in the presence of caffeoyl-CoA. Particularly preferably, the acyl-coenzyme A thioesterase activity may be detected in vivo using a microorganism expressing the enzyme to be tested and capable of converting caffeic acid to feruloyl-CoA and/or of converting p-coumaric acid to caffeoyl-CoA. The acyl-coenzyme A thioesterase activity is then detected via the production of ferulic acid and/or caffeic acid in the presence, respectively, of caffeic acid or p-coumaric acid, preferably by an HPLC technique.

The acyl-coenzyme A thioesterase may be a plant enzyme, preferably an enzyme of plants of the genus Petunia, Oryza, Arabidopsis, Capsella, Camelia, Brassica, Raphanus or Nicotiana, in particular Petunia hybrida, Oryza meyeriana (in particular Oryza meyeriana var. granulata), Arabidopsis thaliana, Capsella rubella, Camelia sativa, Brassica rapa, Raphanus sativus or Nicotiana tabacum. The acyl-coenzyme A thioesterase may also be an enzyme from plants of the genus Petunia, Arabidopsis, Capsella, Camelia, Brassica, Raphanus or Nicotiana, in particular Petunia hybrida, Arabidopsis thaliana, Capsella rubella, Camelia sativa, Brassica rapa, Raphanus sativus or Nicotiana tabacum.

Alternatively, this enzyme may be an enzyme produced by a microorganism, for example by a yeast of the genus Saccharomyces, in particular a Saccharomyces cerevisiae yeast. In certain embodiments, the enzyme corresponds to the endogenous enzyme of the microorganism. In these cases, the enzyme may be overexpressed, for example by replacing the endogenous promoter with a strong heterologous promoter and/or by increasing the number of copies of the gene.

More particularly preferably, the acyl-coenzyme A thioesterase is an enzyme of a plant, in particular of Petunia hybrida, Oryza meyeriana (in particular Oryza meyeriana var. granulata) or Arabidopsis thaliana, more particularly Petunia hybrida or Arabidopsis thaliana. The acyl-coenzyme A thioesterase may be an enzyme of Arabidopsis thaliana, in particular the thioesterase described in SEQ ID NO: 1. Alternatively, the acyl-coenzyme A thioesterase may be an enzyme of Petunia hybrida, in particular the thioesterase described in SEQ ID NO: 2. Alternatively, the acyl-coenzyme A thioesterase may be an enzyme of Oryza meyeriana (in particular Oryza meyeriana var. granulata) in particular the thioesterase described in SEQ ID NO: 39.

According to a particular embodiment, the acyl-coenzyme A thioesterase comprises a sequence chosen from the sequences SEQ ID NOs: 1, 2 and 39, and the polypeptides comprising a sequence having at least 60, 70, 80, 85, 90 or 95% sequence identity with one of these sequences and exhibiting acyl-coenzyme A thioesterase activity. According to a particular embodiment, the acyl-coenzyme A thioesterase comprises a sequence chosen from the sequences SEQ ID NOs: 2 and 39 and the polypeptides comprising a sequence having at least 60, 70, 80, 85, 90 or 95% sequence identity with one of these sequences and exhibiting acyl-coenzyme A thioesterase activity. According to a particular embodiment, the recombinant microorganism according to the invention comprises:
a heterologous nucleic acid sequence coding for a 4CL comprising a sequence chosen from the sequences SEQ ID NOs: 5 to 9 and the polypeptides comprising a sequence having at least 60, 70, 80, 85, 90 or 95% sequence identity with one of these sequences and exhibiting 4CL activity; preferably a 4CL comprising a sequence chosen from the sequences SEQ ID NOs: 5, 6, 8 and 9 and the polypeptides comprising a sequence having at least 60, 70, 80, 85, 90 or 95% sequence identity with one of these sequences and exhibiting 4CL activity, and more particularly preferably a 4CL comprising a sequence chosen from the sequences SEQ ID NOs: 6 and 8 and the polypeptides comprising a sequence having at least 60, 70, 80, 85, 90 or 95% sequence identity with one of these sequences and exhibiting 4CL activity; and
a heterologous nucleic acid sequence coding for a CCoA3H comprising a sequence chosen from the sequences SEQ ID NOs: 42 to 67 and the polypeptides comprising a sequence having at least 60, 70, 80, 85, 90 or 95% sequence identity with one of these sequences and exhibiting CCoA3H activity; preferably a CCoA3H comprising a sequence chosen from the sequences SEQ ID NOs: 42, 45 and 49 and the polypeptides comprising a sequence having at least 60, 70, 80, 85, 90 or 95% sequence identity with one of these sequences and exhibiting CCoA3H activity, and more particularly preferably a CCoA3H comprising a sequence chosen from the sequence SEQ ID NO: 49 and the polypeptides comprising a sequence having at least 60, 70, 80, 85, 90 or 95% sequence identity with said sequence and exhibiting CCoA3H activity; and
a heterologous nucleic acid sequence coding for an acyl-coenzyme A thioesterase comprising a sequence chosen from the sequences SEQ ID NOs: 1, 2 and 39, and the polypeptides comprising a sequence having at least 60, 70, 80, 85, 90 or 95% sequence identity with one of these sequences and exhibiting acyl-coenzyme A thioesterase activity, preferably an acyl-coenzyme A thioesterase comprising a sequence chosen from the sequences SEQ ID NOs: 2 and 39 and the polypeptides comprising a sequence having at least 60, 70, 80, 85, 90 or 95% sequence identity with one of these sequences and exhibiting acyl-coenzyme A thioesterase activity.

The microorganism according to the invention may produce p-coumaric acid, naturally or after genetic modification. Alternatively, this substrate may be supplied to it in the culture medium. According to a particular embodiment, the microorganism is capable of producing p-coumaric acid, from a synthesis intermediate, such as tyrosine, phenylalanine or cinnamic acid, or from glucose via phenylalanine or tyrosine.

Production of Ferulic Acid from p-Coumaric Acid

The production of ferulic acid using a microorganism according to the invention as described above, namely a microorganism expressing a 4CL, a CCoA3H and an acyl-coenzyme A thioesterase, may be obtained:
by direct transformation of caffeic acid into ferulic acid by a caffeic acid O-methyltransferase (COMT), or
by synthesis of feruloyl-CoA from caffeoyl-CoA using a caffeoyl-CoA O-methyltransferase (CCoAMT), ferulic acid then being obtained from the feruloyl-CoA via the activity of an acyl-coenzyme A thioesterase as described above.

Thus, the microorganism according to the invention may also comprise, in addition to 4CL, CCoA3H and acyl-coenzyme A thioesterase, a heterologous nucleic acid sequence coding for a caffeoyl-CoA O-methyltransferase (CCoAMT) and/or a heterologous nucleic acid sequence coding for caffeic acid O-methyltransferase (COMT).

According to one embodiment, the microorganism according to the invention additionally comprises a heterologous nucleic acid sequence coding for a caffeoyl-CoA O-methyltransferase (CCoAMT).

As used herein, the term “caffeoyl-CoA O-methyltransferase” or “CCoAMT” refers to an enzyme belonging to the class EC 2.1.1.104 and which catalyzes the conversion of caffeoyl-CoA to feruloyl-CoA.

The detection of this activity may be achieved by any method known to those skilled in the art, in vivo or in vitro. The CCoAMT activity may be detected in vitro, for example using a commercially available in vitro test (for example the SAM510 test from G-Biosciences, Cat. #786-430). In particular, to determine whether there is any CoA methyltransferase activity, an enzymatic test may be effected consisting of the in vitro incubation of a mixture composed of the enzyme to be tested (putative CCoAMT), S-adenosylmethionine (SAM), and a mixture of enzymes (5-adenosylhomocysteine nucleosidase (EC 3.2.2.9), adenine deaminase (EC 3.5.4.2) and xanthine oxidase (EC 1.17.3.2)) under optimal conditions (pH, temperature, ions, etc.). In the presence of a CCoAMT activity, the products obtained are urate and hydrogen peroxide. After a certain incubation time, the appearance of hydrogen peroxide can be detected by reaction with a colorimetric agent, namely 3,5-dichloro-2-hydroxybenzenesulfonic acid (DHBS) and measured in UV spectrophotometry at 510 nm. The CCoAMT activity may also be detected in vivo, in particular using the method described in the experimental part hereinafter. In particular, the CCoAMT activity may be detected in vivo using a microorganism expressing the enzyme to be tested. The substrate of said enzyme (caffeoyl-CoA) is either synthesized by the microorganism or supplied to the culture medium. The feruloyl-CoA produced by an enzyme exhibiting the desired activity may then be detected by any method, preferably by an HPLC technique. Particularly preferably, the CCoAMT activity may be detected in vivo using a microorganism expressing the enzyme to be tested and capable of converting p-coumaric acid to caffeoyl-CoA, that is to say expressing a 4CL and a CCoA3H as defined above. The CCoAMT activity is detected via the production of feruloyl-CoA in the presence of p-coumaric acid, preferably by an HPLC technique.

The CCoAMT may be a plant enzyme, preferably of a plant of the genus Vitis, Medicago, Eucalyptus. Nicotiana. Arabidopsis. Panicum. Rauvolfia or Populus, and more particularly preferably of the genus Vitis, Medicago, Eucalyptus, Nicotiana, Arabidopsis or Populus. In particular, the CCoAMT may be an enzyme of Vitis vinifera, Medicago sativa, Eucalyptus globus, Nicotiana tabacum, Arabidopsis thaliana, Panicum virgatum, Rauvolfia serpentina or Populus trichocarpa, preferably an enzyme of Vitis vinifera, Medicago sativa, Eucalyptus globus, Nicotiana tabacum, Arabidopsis thaliana or Populus trichocarpa.

The CCoAMT may be an enzyme of Vitis vinifera, in particular the CCoAMT described in SEQ ID NO: 10, an enzyme of Medicago sativa, in particular the CCoAMT described in SEQ ID NO: 11, an enzyme of Eucalyptus globus, in particular the CCoAMT described in SEQ ID NO: 12, an enzyme of Nicotiana tabacum, in particular the CCoAMT described in SEQ ID NO: 13 or 14, an enzyme of Arabidopsis thaliana, in particular the CCoAMT described in SEQ ID NO: an enzyme of Populus trichocarpa, in particular the CCoAMT described in SEQ ID NO: 16, an enzyme of Panicum virgatum, in particular the CCoAMT described in SEQ ID NO: 40 or an enzyme of Rauvolfia serpentina, in particular the CCoAMT described in SEQ ID NO: 41.

According to one embodiment, the CCoAMT comprises a sequence chosen from the sequences SEQ ID NOs: 10 to 16, 40 and 41, and the polypeptides comprising a sequence having at least 60, 70, 80, 85, 90 or 95% sequence identity with one of these sequences and exhibiting CCoAMT activity.

According to a particular embodiment, the CCoAMT comprises a sequence chosen from the sequence SEQ ID NO: 40 and the polypeptides comprising a sequence having at least 60, 70, 80, 85, 90 or 95% sequence identity with said sequence and exhibiting CCoAMT activity.

According to a particular embodiment, the recombinant microorganism according to the invention comprises:
a heterologous nucleic acid sequence coding for a 4CL comprising a sequence chosen from the sequences SEQ ID NOs: 5 to 9 and the polypeptides comprising a sequence having at least 60, 70, 80, 85, 90 or 95% sequence identity with one of these sequences and exhibiting 4CL activity; preferably a 4CL comprising a sequence chosen from the sequences SEQ ID NOs: 5, 6, 8 and 9 and the polypeptides comprising a sequence having at least 60, 70, 80, 85, 90 or 95% sequence identity with one of these sequences and exhibiting 4CL activity, and more particularly preferably a 4CL comprising a sequence chosen from the sequences SEQ ID NOs: 6 and 8 and the polypeptides comprising a sequence having at least 60, 70, 80, 85, 90 or 95% sequence identity with one of these sequences and exhibiting 4CL activity; and
a heterologous nucleic acid sequence coding for a CCoA3H comprising a sequence chosen from the sequences SEQ ID NOs: 42 to 67 and the polypeptides comprising a sequence having at least 60, 70, 80, 85, 90 or 95% sequence identity with one of these sequences and exhibiting CCoA3H activity; preferably a CCoA3H comprising a sequence chosen from the sequences SEQ ID NOs: 42, 45 and 49 and the polypeptides comprising a sequence having at least 60, 70, 80, 85, 90 or 95% sequence identity with one of these sequences and exhibiting CCoA3H activity, and more particularly preferably a CCoA3H comprising a sequence chosen from the sequence SEQ ID NO: 49 and the polypeptides comprising a sequence having at least 60, 70, 80, 85, 90 or 95% sequence identity with said sequence and exhibiting CCoA3H activity; and
a heterologous nucleic acid sequence coding for an acyl-coenzyme A thioesterase comprising a sequence chosen from the sequences SEQ ID NOs: 1, 2 and 39, and the polypeptides comprising a sequence having at least 60, 70, 80, 85, 90 or 95% sequence identity with one of these sequences and exhibiting acyl-coenzyme A thioesterase activity, preferably an acyl-coenzyme A thioesterase comprising a sequence chosen from the sequences SEQ ID NOs: 2 and 39 and the polypeptides comprising a sequence having at least 60, 70, 80, 85, 90 or 95% sequence identity with one of these sequences and exhibiting acyl-coenzyme A thioesterase activity; and
a heterologous nucleic acid sequence coding for a CCoAMT comprising a sequence chosen from the sequences SEQ ID NOs: 10 to 16, 40 and 41 and the polypeptides comprising a sequence having at least 60, 70, 80, 85, 90 or 95% sequence identity with one of these sequences and exhibiting CCoAMT activity, preferably a CCoAMT comprising a sequence chosen from the sequence SEQ ID NO: 40 and the polypeptides comprising a sequence having at least 60, 70, 80, 85, 90 or 95% sequence identity with said sequence and exhibiting CCoAMT activity.

According to a preferred embodiment, the recombinant microorganism comprises
a heterologous nucleic acid sequence coding for a 4CL comprising a sequence chosen from the sequences SEQ ID NOs: 6 and 8 and the polypeptides comprising a sequence having at least 60, 70, 80, 85, 90 or 95% sequence identity with one of these sequences and exhibiting 4CL activity; and
a heterologous nucleic acid sequence coding for a CCoA3H comprising a sequence chosen from the sequences SEQ ID NOs: 42, 45 and 49 and the polypeptides comprising a sequence having at least 60, 70, 80, 85, 90 or 95% sequence identity with one of these sequences and exhibiting CCoA3H activity, and preferably a CCoA3H comprising a sequence chosen from the sequence SEQ ID NO: 49 and the polypeptides comprising a sequence having at least 60, 70, 80, 85, 90 or 95% sequence identity with said sequence and exhibiting CCoA3H activity; and
a heterologous nucleic acid sequence coding for an acyl-coenzyme A thioesterase comprising a sequence chosen from the sequences SEQ ID NOs: 2 and 39 and the polypeptides comprising a sequence having at least 60, 70, 80, 85, 90 or 95% sequence identity with one of these sequences and exhibiting acyl-coenzyme A thioesterase activity; and
a heterologous nucleic acid sequence coding for a CCoAMT comprising a sequence chosen from the sequence SEQ ID NO: 40 and the polypeptides comprising a sequence having at least 60, 70, 80, 85, 90 or 95% sequence identity with said sequence and exhibiting CCoAMT activity.

In the embodiments in which the microorganism according to the invention comprises a heterologous nucleic acid sequence coding for a CCoAMT, said microorganism may additionally comprise a heterologous nucleic acid sequence coding for a cinnamoyl-CoA reductase (CCR) and a heterologous nucleic acid sequence coding for an aldehyde dehydrogenase (ALDH).

CCR catalyzes the reduction of carboxyl-CoAs such as feruloyl-CoA, thus forming aldehydes such as coniferaldehyde. ALDH then catalyzes the oxidation of the aldehydes thus formed to carboxylic acids such as ferulic acid.

As used herein, the term “cinnamoyl-CoA reductase” or “CCR” refers to an enzyme belonging to the class EC 1.2.1.44 and which catalyzes the reduction of a substituted cinnamoyl-CoA, for example feruloyl-CoA, to a corresponding cinnamaldehyde, for example coniferaldehyde. Preferably, this term refers to an enzyme which catalyzes the reduction of feruloyl-CoA to coniferaldehyde.

The detection of this activity may be achieved by any method known to those skilled in the art, in vivo or in vitro. The CCR activity may be detected in vitro, for example as described in the article by Chao et al. (Planta. 2017 January; 245(1): 61-75). In particular, in order to determine whether there is any cinnamoyl-CoA reductase activity, an enzymatic test may be effected consisting of the in vitro incubation of a mixture composed of the enzyme to be tested (putative CCR), a carboxyl-CoA substrate and NADPH under optimal conditions (pH, temperature, ions, etc.). After a certain incubation time, the appearance of the aldehyde form can be observed either in UV spectrophotometry with a given wavelength or by HPLC. The CCR activity may also be detected in vivo, in particular using the method described in the experimental part hereinafter. In particular, the CCR activity may be detected in vivo using a microorganism expressing the enzyme to be tested. The carboxyl-CoA substrate of said enzyme is either synthesized by the microorganism or supplied to the culture medium. The aldehyde produced by an enzyme exhibiting the desired activity may then be detected by any method, preferably by an HPLC technique. Particularly preferably, the CCR activity may be detected in vivo using a microorganism expressing the enzyme to be tested and capable of converting caffeic acid or p-coumaric acid to feruloyl-CoA and capable of converting coniferaldehyde to ferulic acid, that is to say expressing an ALDH enzyme as defined above. The CCR activity is detected via the production of ferulic acid in the presence of caffeic acid or p-coumaric acid, preferably by an HPLC technique.

The CCR may be a plant enzyme, preferably of a plant of the genus Populus, Arabidopsis, Oryza, Zea, Medicago or Sorghum, in particular Populus tomentosa, Arabidopsis thaliana, Oryza sativa, Zea Mays, Medicago truncatula or Sorghum bicolor. Preferably, the CCR is an enzyme of Populus tomentosa, in particular the CCR described in SEQ ID NO: 4. According to a preferred embodiment, the CCR comprises a sequence chosen from the sequence SEQ ID NO: 4 and the polypeptides comprising a sequence having at least 60, 70, 80, 85, 90 or 95% sequence identity with the sequence SEQ ID NO: 4 and exhibiting CCR activity.

As used herein, the term “aldehyde dehydrogenase” or “ALDH” refers to an enzyme belonging to the class EC 1.2.1.3 and which catalyzes the oxidation of an aldehyde, for example coniferaldehyde, to carboxylic acid, for example to ferulic acid. Preferably, this term refers to an enzyme which catalyzes the oxidation of coniferaldehyde to ferulic acid. The detection of this activity may be achieved by any method known to those skilled in the art, in vivo or in vitro. The ALDH activity may be detected in vitro, for example using a commercially available in vitro test kit. In particular, in order to determine whether there is any aldehyde dehydrogenase activity, an enzymatic test may be effected consisting of the in vitro incubation of a mixture composed of the enzyme to be tested (putative ALDH), an aldehyde and NAD under optimal conditions (pH, temperature, ions, etc.). After a certain incubation time, the appearance of the acid form and NADH can be observed either in UV spectrophotometry at 450 nm or by HPLC. The ALDH activity may also be detected in vivo, in particular using the method described in the experimental part hereinafter. In particular, the ALDH activity may be detected in vivo using a microorganism expressing the enzyme to be tested. The aldehyde substrate of said enzyme is either synthesized by the microorganism or supplied to the culture medium. The carboxylic acid produced by an enzyme exhibiting the desired activity may then be detected by any method, preferably by an HPLC technique. Particularly preferably, the ALDH activity may be detected in vivo using a microorganism expressing the enzyme to be tested and capable of converting caffeic acid or p-coumaric acid to feruloyl-CoA and capable of converting feruloyl-CoA to coniferaldehyde, that is to say expressing a CCR enzyme as defined above. The ALDH activity is detected via the production of ferulic acid in the presence of caffeic acid or p-coumaric acid, preferably by an HPLC technique.

The ALDH may be a plant enzyme, preferably of a plant of the genus Arabidopsis, Populus, Oryza, Zea, Medicago or Sorghum, in particular Arabidopsis thaliana, Populus tomentosa, Oryza sativa, Zea Mays, Medicago truncatula or Sorghum bicolor.

Alternatively, this enzyme may be an enzyme produced by a microorganism, for example by a yeast of the genus Saccharomyces, in particular a Saccharomyces cerevisiae yeast. In certain embodiments, the enzyme corresponds to the endogenous enzyme of the microorganism. In these cases, the enzyme may be overexpressed, for example by replacing the endogenous promoter with a strong heterologous promoter and/or by increasing the number of copies of the gene.

Preferably, the ALDH is a plant enzyme, and more particularly preferably an enzyme of Arabidopsis thaliana, in particular the ALDH described in SEQ ID NO: 3.

According to a particular embodiment, the ALDH comprises a sequence chosen from the sequence SEQ ID NO: 3 and the polypeptides comprising a sequence having at least 60, 70, 80, 85, 90 or 95% sequence identity with the sequence SEQ ID NO: 3 and exhibiting ALDH activity.

According to a particular embodiment, the microorganism according to the invention comprises a heterologous nucleic acid sequence coding for a CCR and a heterologous nucleic acid sequence coding for an ALDH and in which
said CCR comprises a sequence chosen from the sequence SEQ ID NO: 4 and the polypeptides comprising a sequence having at least 60, 70, 80, 85, 90 or 95% sequence identity with the sequence SEQ ID NO: 4 and exhibiting CCR activity; and
said ALDH comprises a sequence chosen from the sequence SEQ ID NO: 3 and the polypeptides comprising a sequence having at least 60, 70, 80, 85, 90 or 95% sequence identity with the sequence SEQ ID NO: 3 and exhibiting ALDH activity.

According to particular embodiments, the microorganism according to the invention comprises a heterologous nucleic acid sequence coding for a 4CL as defined above, a heterologous nucleic acid sequence coding for a CCoA3H as defined above, a heterologous nucleic acid sequence coding for an acyl-coenzyme A thioesterase as defined above, a heterologous nucleic acid sequence coding for a CCoAMT as defined above and optionally comprises a heterologous nucleic acid sequence coding for a CCR and a heterologous nucleic acid sequence coding for an ALDH as defined above.

According to a particular embodiment, the recombinant microorganism according to the invention comprises:
a heterologous nucleic acid sequence coding for a 4CL comprising a sequence chosen from the sequences SEQ ID NOs: 5 to 9 and the polypeptides comprising a sequence having at least 60, 70, 80, 85, 90 or 95% sequence identity with one of these sequences and exhibiting 4CL activity; preferably a 4CL comprising a sequence chosen from the sequences SEQ ID NOs: 5, 6, 8 and 9 and the polypeptides comprising a sequence having at least 60, 70, 80, 85, 90 or 95% sequence identity with one of these sequences and exhibiting 4CL activity, and more particularly preferably a 4CL comprising a sequence chosen from the sequences SEQ ID NOs: 6 and 8 and the polypeptides comprising a sequence having at least 60, 70, 80, 85, 90 or 95% sequence identity with one of these sequences and exhibiting 4CL activity; and
a heterologous nucleic acid sequence coding for a CCoA3H comprising a sequence chosen from the sequences SEQ ID NOs: 42 to 67 and the polypeptides comprising a sequence having at least 60, 70, 80, 85, 90 or 95% sequence identity with one of these sequences and exhibiting CCoA3H activity; preferably a CCoA3H comprising a sequence chosen from the sequences SEQ ID NOs: 42, 45 and 49 and the polypeptides comprising a sequence having at least 60, 70, 80, 85, 90 or 95% sequence identity with one of these sequences and exhibiting CCoA3H activity, and more particularly preferably a CCoA3H comprising a sequence chosen from the sequence SEQ ID NO: 49 and the polypeptides comprising a sequence having at least 60, 70, 80, 85, 90 or 95% sequence identity with said sequence and exhibiting CCoA3H activity; and
a heterologous nucleic acid sequence coding for an acyl-coenzyme A thioesterase comprising a sequence chosen from the sequences SEQ ID NOs: 1, 2 and 39, and the polypeptides comprising a sequence having at least 60, 70, 80, 85, 90 or 95% sequence identity with one of these sequences and exhibiting acyl-coenzyme A thioesterase activity, preferably an acyl-coenzyme A thioesterase comprising a sequence chosen from the sequences SEQ ID NOs: 2 and 39 and the polypeptides comprising a sequence having at least 60, 70, 80, 85, 90 or 95% sequence identity with one of these sequences and exhibiting acyl-coenzyme A thioesterase activity; and
a heterologous nucleic acid sequence coding for a CCoAMT comprising a sequence chosen from the sequences SEQ ID NOs: 10 to 16, 40 and 41 and the polypeptides comprising a sequence having at least 60, 70, 80, 85, 90 or 95% sequence identity with one of these sequences and exhibiting CCoAMT activity, preferably a CCoAMT comprising a sequence chosen from the sequence SEQ ID NO: 40 and the polypeptides comprising a sequence having at least 60, 70, 80, 85, 90 or 95% sequence identity with said sequence and exhibiting CCoAMT activity; and
a heterologous nucleic acid sequence coding for a CCR comprising a sequence chosen from the sequence SEQ ID NO: 4 and the polypeptides comprising a sequence having at least 60, 70, 80, 85, 90 or 95% sequence identity with the sequence SEQ ID NO: 4 and exhibiting CCR activity; and
a heterologous nucleic acid sequence coding for an ALDH comprising a sequence chosen from the sequence SEQ ID NO: 3 and the polypeptides comprising a sequence having at least 60, 70, 80, 85, 90 or 95% sequence identity with the sequence SEQ ID NO: 3 and exhibiting ALDH activity.

Alternatively or in addition to the presence of CCoAMT, the microorganism according to the invention may also comprise a heterologous nucleic acid sequence coding for a caffeic acid O-methyltransferase (COMT).

As used herein, the term “caffeic acid O-methyltransferase” or “COMT” refers to an enzyme belonging to the class EC 2.1.1.68 and which catalyzes the conversion of caffeic acid to ferulic acid.

The detection of this activity may be achieved by any method known to those skilled in the art, in vivo or in vitro. The COMT activity may be detected in vitro, for example using a commercially available in vitro test (for example the Methyltransferase activity kit test, Enzo Life Sciences, AFI-907-025). The COMT activity may also be detected in vivo, in particular using the method described in the experimental part hereinafter. In particular, the COMT activity may be detected in vivo using a microorganism expressing the enzyme to be tested. The substrate of said enzyme (caffeic acid) is either synthesized by the microorganism or supplied to the culture medium. The ferulic acid produced by an enzyme exhibiting the desired activity may then be detected by any method, preferably by an HPLC technique. Particularly preferably, the COMT activity may be detected in vivo using a microorganism expressing the enzyme to be tested and capable of converting p-coumaric acid to caffeic acid and not possessing any other pathway enabling the production of ferulic acid. The COMT activity is detected via the production of ferulic acid in the presence of p-coumaric acid, preferably by an HPLC technique.

The COMT may be a plant enzyme, preferably of a plant of the genus Panicum, Arabidopsis, Catharanthus, Triticum, Nicotiana, Picea, Zea, Saccharum, Stylosanthes, Cucumis, Tarenaya, Ziziphus, Cucurbita, Ipomoea, Thalictrum, Punica, Brassica, Lycium or Acer, more particularly preferably of the genus Panicum, Arabidopsis, Catharanthus, Triticum, Nicotiana, Picea, Zea, Picea or Saccharum.

In particular, the COMT may be an enzyme of Panicum virgatum, Arabidopsis thaliana, Catharanthus roseus, Triticum aestivum, Nicotiana tabacum, Picea abies, Zea mays, Stylosanthes humilis, Saccharum officinarum, Cucumis sativus, Tarenaya hassleriana, Ziziphus jujuba var. spinosa, Cucurbita maxima, Ipomoea nil, Thalictrum tuberosum, Punica granatum, Brassica cretica, Lycium chinense or Acer yangbiense, preferably an enzyme of Panicum virgatum, Arabidopsis thaliana, Catharanthus roseus, Triticum aestivum, Nicotiana tabacum, Picea abies, Zea mays, Stylosanthes humilis or Saccharum officinarum.

In particular, the COMT may be an enzyme of Panicum virgatum, in particular the COMT described in SEQ ID NO: 71 or 76, an enzyme of Arabidopsis thaliana, in particular the COMT described in SEQ ID NO: 72, an enzyme of Catharanthus roseus, in particular the COMT described in SEQ ID NO: 73, an enzyme of Triticum aestivum, in particular the COMT described in SEQ ID NO: 74 or 75, an enzyme of Nicotiana tabacum, in particular the COMT described in SEQ ID NO: 77, an enzyme of Picea abies, in particular the COMT described in SEQ ID NO: 78, an enzyme of Zea mays, in particular the COMT described in SEQ ID NO: 79 or in SEQ ID NO: 82, an enzyme of Stylosanthes humilis, in particular the COMT described in SEQ ID NO: 80, an enzyme of Saccharum officinarum, in particular the COMT described in SEQ ID NO: 81, an enzyme of Cucumis sativus, in particular the COMT described in SEQ ID NO: 83, an enzyme of Tarenaya hassleriana, in particular the COMT described in SEQ ID NO: 84, an enzyme of Ziziphus jujuba var. spinosa, in particular the COMT described in SEQ ID NO: 85, an enzyme of Cucurbita maxima, in particular the COMT described in SEQ ID NO: 86, an enzyme of/pomoea nil, in particular the COMT described in SEQ ID NO: 87, an enzyme of Thalictrum tuberosum, in particular the COMT described in SEQ ID NO: 88, an enzyme of Punica granatum, in particular the COMT described in SEQ ID NO: 89, an enzyme of Brassica cretica, in particular the COMT described in SEQ ID NO: 90, an enzyme of Lycium chinense, in particular the COMT described in SEQ ID NO: 91 or an enzyme of Acer yangbiense, in particular the COMT described in SEQ ID NO: 92.

More particularly, the COMT may be an enzyme of Panicum virgatum, in particular the COMT described in SEQ ID NO: 71 or 76, an enzyme of Arabidopsis thaliana, in particular the COMT described in SEQ ID NO: 72, an enzyme of Catharanthus roseus, in particular the COMT described in SEQ ID NO: 73, an enzyme of Triticum aestivum, in particular the COMT described in SEQ ID NO: 74 or 75, an enzyme of Nicotiana tabacum, in particular the COMT described in SEQ ID NO: 77, an enzyme of Picea abies, in particular the COMT described in SEQ ID NO: 78, an enzyme of Zea mays, in particular the COMT described in SEQ ID NO: 79, an enzyme of Stylosanthes humilis, in particular the COMT described in SEQ ID NO: 80, or an enzyme of Saccharum officinarum, in particular the COMT described in SEQ ID NO: 81. According to one embodiment, the COMT comprises a sequence chosen from the sequences SEQ ID NOs: 71 to 92, and the polypeptides comprising a sequence having at least 60, 70, 80, 85, 90 or 95% sequence identity with one of these sequences and exhibiting COMT activity.

According to a particular embodiment, the COMT comprises a sequence chosen from the sequences SEQ ID NOs: 71 to 81, and the polypeptides comprising a sequence having at least 60, 70, 80, 85, 90 or 95% sequence identity with one of these sequences and exhibiting COMT activity.

According to a preferred embodiment, the COMT comprises a sequence chosen from the sequences SEQ ID NOs: 72 to 74, 76 and 79 to 92 and the polypeptides comprising a sequence having at least 60, 70, 80, 85, 90 or 95% sequence identity with one of these sequences and exhibiting COMT activity.

According to a further preferred embodiment, the COMT comprises a sequence chosen from the sequences SEQ ID NOs: 72, 74, 76, 79, 80 and 82 and the polypeptides comprising a sequence having at least 60, 70, 80, 85, 90 or 95% sequence identity with one of these sequences and exhibiting COMT activity.

According to a particular embodiment, the COMT comprises a sequence chosen from the sequence SEQ ID NO: 72 and the polypeptides comprising a sequence having at least 60, 70, 80, 85, 90 or 95% sequence identity with said sequence and exhibiting COMT activity. According to a particular embodiment, the recombinant microorganism according to the invention comprises:
a heterologous nucleic acid sequence coding for a 4CL comprising a sequence chosen from the sequences SEQ ID NOs: 5 to 9 and the polypeptides comprising a sequence having at least 60, 70, 80, 85, 90 or 95% sequence identity with one of these sequences and exhibiting 4CL activity; preferably a 4CL comprising a sequence chosen from the sequences SEQ ID NOs: 5, 6, 8 and 9 and the polypeptides comprising a sequence having at least 60, 70, 80, 85, 90 or 95% sequence identity with one of these sequences and exhibiting 4CL activity, and more particularly preferably a 4CL comprising a sequence chosen from the sequences SEQ ID NOs: 6 and 8 and the polypeptides comprising a sequence having at least 60, 70, 80, 85, 90 or 95% sequence identity with one of these sequences and exhibiting 4CL activity; and
a heterologous nucleic acid sequence coding for a CCoA3H comprising a sequence chosen from the sequences SEQ ID NOs: 42 to 67 and the polypeptides comprising a sequence having at least 60, 70, 80, 85, 90 or 95% sequence identity with one of these sequences and exhibiting CCoA3H activity; preferably a CCoA3H comprising a sequence chosen from the sequences SEQ ID NOs: 42, 45 and 49 and the polypeptides comprising a sequence having at least 60, 70, 80, 85, 90 or 95% sequence identity with one of these sequences and exhibiting CCoA3H activity, and more particularly preferably a CCoA3H comprising a sequence chosen from the sequence SEQ ID NO: 49 and the polypeptides comprising a sequence having at least 60, 70, 80, 85, 90 or 95% sequence identity with said sequence and exhibiting CCoA3H activity; and
a heterologous nucleic acid sequence coding for an acyl-coenzyme A thioesterase comprising a sequence chosen from the sequences SEQ ID NOs: 1, 2 and 39, and the polypeptides comprising a sequence having at least 60, 70, 80, 85, 90 or 95% sequence identity with one of these sequences and exhibiting acyl-coenzyme A thioesterase activity, preferably an acyl-coenzyme A thioesterase comprising a sequence chosen from the sequences SEQ ID NOs: 2 and 39 and the polypeptides comprising a sequence having at least 60, 70, 80, 85, 90 or 95% sequence identity with one of these sequences and exhibiting acyl-coenzyme A thioesterase activity; and
a heterologous nucleic acid sequence coding for a COMT comprising a sequence chosen from the sequences SEQ ID NOs: 71 to 92, preferably SEQ ID NOs: 71 to 81, and the polypeptides comprising a sequence having at least 60, 70, 80, 85, 90 or 95% sequence identity with one of these sequences, preferably with one of the sequences SEQ ID NOs: 71 to 81, and exhibiting COMT activity, preferably a COMT comprising a sequence chosen from the sequences SEQ ID NOs: 72 to 74, 76 and 79 to 92, preferably the sequences SEQ ID NOs: 72, 74, 76, 79, 80 and 82, and the polypeptides comprising a sequence having at least 60, 70, 80, 85, 90 or 95% sequence identity with one of these sequences, preferably with one of the sequences SEQ ID NOs: 72, 74, 76, 79, 80 and 82, and exhibiting COMT activity, and more particularly preferably a COMT comprising a sequence chosen from the sequence SEQ ID NO: 72 and the polypeptides comprising a sequence having at least 60, 70, 80, 85, 90 or 95% sequence identity with said sequence and exhibiting COMT activity.

Optionally, the microorganism according to the invention may also comprise a heterologous nucleic acid sequence coding for a CCoAMT comprising a sequence chosen from the sequences SEQ ID NOs: 10 to 16, 40 and 41 and the polypeptides comprising a sequence having at least 60, 70, 80, 85, 90 or 95% sequence identity with one of these sequences and exhibiting CCoAMT activity, preferably a CCoAMT comprising a sequence chosen from the sequence SEQ ID NO: 40 and the polypeptides comprising a sequence having at least 60, 70, 80, 85, 90 or 95% sequence identity with said sequence and exhibiting CCoAMT activity. According to a preferred embodiment, the recombinant microorganism comprises
a heterologous nucleic acid sequence coding for a 4CL comprising a sequence chosen from the sequences SEQ ID NOs: 6 and 8 and the polypeptides comprising a sequence having at least 60, 70, 80, 85, 90 or 95% sequence identity with one of these sequences and exhibiting 4CL activity; and
a heterologous nucleic acid sequence coding for a CCoA3H comprising a sequence chosen from the sequences SEQ ID NOs: 42, 45 and 49 and the polypeptides comprising a sequence having at least 60, 70, 80, 85, 90 or 95% sequence identity with one of these sequences and exhibiting CCoA3H activity, and preferably a CCoA3H comprising a sequence chosen from the sequence SEQ ID NO: 49 and the polypeptides comprising a sequence having at least 60, 70, 80, 85, 90 or 95% sequence identity with said sequence and exhibiting CCoA3H activity; and
a heterologous nucleic acid sequence coding for an acyl-coenzyme A thioesterase comprising a sequence chosen from the sequences SEQ ID NOs: 2 and 39 and the polypeptides comprising a sequence having at least 60, 70, 80, 85, 90 or 95% sequence identity with one of these sequences and exhibiting acyl-coenzyme A thioesterase activity; and
a heterologous nucleic acid sequence coding for a COMT comprising a sequence chosen from the sequences SEQ ID NOs: 72, 74, 76, 79, 80 and 82, and the polypeptides comprising a sequence having at least 60, 70, 80, 85, 90 or 95% sequence identity with one of these sequences and exhibiting COMT activity, preferably comprising a sequence chosen from the sequence SEQ ID NO: 72 and the polypeptides comprising a sequence having at least 60, 70, 80, 85, 90 or 95% sequence identity with said sequence and exhibiting COMT activity. Optionally, the microorganism according to the invention may also comprise a heterologous nucleic acid sequence coding for a CCoAMT comprising a sequence chosen from the sequence SEQ ID NO: 40 and the polypeptides comprising a sequence having at least 60, 70, 80, 85, 90 or 95% sequence identity with said sequence and exhibiting CCoAMT activity. The microorganism according to the invention may be capable of producing p-coumaric acid from a synthesis intermediate such as cinnamic acid, tyrosine or phenylalanine, or from glucose via phenylalanine or tyrosine.
   Production of p-Coumaric Acid from Tyrosine

The production of p-coumaric acid from tyrosine involves an enzyme exhibiting a tyrosine ammonia lyase (TAL) activity.

Thus, the microorganism according to the invention may comprise, in addition to the heterologous nucleic acid sequences described above and coding for a 4CL, a CCoA3H, an acyl-coenzyme A thioesterase and optionally for a COMT and/or a CCoAMT (optionally in combination with a CCR and an ALDH), a heterologous nucleic acid sequence coding for an enzyme having a tyrosine ammonia lyase activity.

As used herein, the term “tyrosine ammonia lyase” or “TAL” refers to an enzyme which catalyzes the production of p-coumaric acid from L-tyrosine (EC 4.3.1.23). The detection of a tyrosine ammonia lyase activity may be achieved by any method known to those skilled in the art, in vivo or in vitro. The TAL activity may in particular be detected via an enzymatic test consisting of the in vitro incubation of a mixture composed of the enzyme to be tested and tyrosine under optimal conditions (pH, temperature, ions, etc.). After a certain incubation time, the appearance of p-coumaric acid is observed in UPLC-MS in comparison with the expected standard.

Certain TALs may also exhibit a dihydroxyphenylalanine ammonia lyase (DAL) and/or phenylalanine ammonia lyase (PAL) activity.

The TAL may be an enzyme produced by a bacterium, in particular a bacterium of the genus Rhodobacter, preferably Rhodobacter capsulatus or Rhodobacter sphaeroides, of the genus Ralstonia, preferably Ralstonia metallidurans, or of the genus Flavobacteriaceae, preferably Flavobacteriumjohnsoniae. It may also be an enzyme produced by a plant, for example by Citrus sinensis, Camellia sinensis, Fragaria x ananassa or Zea mays. Preferably, the TAL is an enzyme produced by a yeast, in particular a yeast of the genus Rhodotorula, for example Rhodotorula glutinis or by a plant, in particular of the genus Citrus, for example Citrus sinensis.

In particular, the TAL may be an enzyme of Flavobacterium johnsoniae, preferably the enzyme described in SEQ ID NO: 30, an enzyme of Rhodotorula glutinis, preferably the enzyme described in SEQ ID NO: 19 or an enzyme of Citrus sinensis, preferably the enzyme described in SEQ ID NO: 68.

In a particular embodiment, the TAL comprises a sequence chosen from SEQ ID NOs: 19, 30 and 68 and the polypeptides comprising a sequence having at least 60, 70, 80, 85, 90 or 95% sequence identity with one of these sequences and exhibiting TAL activity.

In a preferred embodiment, the TAL comprises a sequence chosen from SEQ ID NOs: 19 and 68 and the polypeptides comprising a sequence having at least 60, 70, 80, 85, 90 or 95% sequence identity with one of these sequences and exhibiting TAL activity.

In a further preferred embodiment, the TAL comprises a sequence chosen from SEQ ID NO: 19 and the polypeptides comprising a sequence having at least 60, 70, 80, 85, 90 or 95% sequence identity with the sequence SEQ ID NO: 19 and exhibiting TAL activity.

According to a particular embodiment, the recombinant microorganism according to the invention comprises:
a heterologous nucleic acid sequence coding for a 4CL comprising a sequence chosen from the sequences SEQ ID NOs: 5 to 9 and the polypeptides comprising a sequence having at least 60, 70, 80, 85, 90 or 95% sequence identity with one of these sequences and exhibiting 4CL activity; preferably a 4CL comprising a sequence chosen from the sequences SEQ ID NOs: 5, 6, 8 and 9 and the polypeptides comprising a sequence having at least 60, 70, 80, 85, 90 or 95% sequence identity with one of these sequences and exhibiting 4CL activity, and more particularly preferably a 4CL comprising a sequence chosen from the sequences SEQ ID NOs: 6 and 8 and the polypeptides comprising a sequence having at least 60, 70, 80, 85, 90 or 95% sequence identity with one of these sequences and exhibiting 4CL activity; and
a heterologous nucleic acid sequence coding for a CCoA3H comprising a sequence chosen from the sequences SEQ ID NOs: 42 to 67 and the polypeptides comprising a sequence having at least 60, 70, 80, 85, 90 or 95% sequence identity with one of these sequences and exhibiting CCoA3H activity; preferably a CCoA3H comprising a sequence chosen from the sequences SEQ ID NOs: 42, 45 and 49 and the polypeptides comprising a sequence having at least 60, 70, 80, 85, 90 or 95% sequence identity with one of these sequences and exhibiting CCoA3H activity, and more particularly preferably a CCoA3H comprising a sequence chosen from the sequence SEQ ID NO: 49 and the polypeptides comprising a sequence having at least 60, 70, 80, 85, 90 or 95% sequence identity with said sequence and exhibiting CCoA3H activity; and
a heterologous nucleic acid sequence coding for an acyl-coenzyme A thioesterase comprising a sequence chosen from the sequences SEQ ID NOs: 1, 2 and 39, and the polypeptides comprising a sequence having at least 60, 70, 80, 85, 90 or 95% sequence identity with one of these sequences and exhibiting acyl-coenzyme A thioesterase activity, preferably an acyl-coenzyme A thioesterase comprising a sequence chosen from the sequences SEQ ID NOs: 2 and 39 and the polypeptides comprising a sequence having at least 60, 70, 80, 85, 90 or 95% sequence identity with one of these sequences and exhibiting acyl-coenzyme A thioesterase activity; and
a heterologous nucleic acid sequence coding for a TAL comprising a sequence chosen from the sequences SEQ ID NOs: 19, 30 and 68 and the polypeptides comprising a sequence having at least 60, 70, 80, 85, 90 or 95% sequence identity with one of these sequences and exhibiting TAL activity, preferably a TAL comprising a sequence chosen from the sequences SEQ ID NOs: 19 and 68, preferably SEQ ID NO: 19, and the polypeptides comprising a sequence having at least 60, 70, 80, 85, 90 or 95% sequence identity with one of these sequences, preferably with SEQ ID NO: 19, and exhibiting TAL activity.

Optionally, in this embodiment, the microorganism may also comprise
a heterologous nucleic acid sequence coding for a CCoAMT comprising a sequence chosen from the sequences SEQ ID NOs: 10 to 16, 40 and 41 and the polypeptides comprising a sequence having at least 60, 70, 80, 85, 90 or 95% sequence identity with one of these sequences and exhibiting CCoAMT activity, preferably a CCoAMT comprising a sequence chosen from the sequence SEQ ID NO: 40 and the polypeptides comprising a sequence having at least 60, 70, 80, 85, 90 or 95% sequence identity with said sequence and exhibiting CCoAMT activity; and/or
a heterologous nucleic acid sequence coding for a COMT comprising a sequence chosen from the sequences SEQ ID NOs: 71 to 92, preferably SEQ ID NOs: 71 to 81, and the polypeptides comprising a sequence having at least 60, 70, 80, 85, 90 or 95% sequence identity with one of these sequences, preferably with one of the sequences SEQ ID NOs: 71 to 81, and exhibiting COMT activity, preferably a COMT comprising a sequence chosen from the sequences SEQ ID NOs: 72 to 74, 76 and 79 to 92, preferably the sequences SEQ ID NOs: 72, 74, 76, 79, 80 and 82, and the polypeptides comprising a sequence having at least 60, 70, 80, 85, 90 or 95% sequence identity with one of these sequences, preferably with one of the sequences SEQ ID NOs: 72, 74, 76, 79, 80 and 82, and exhibiting COMT activity, and more particularly preferably a COMT comprising a sequence chosen from the sequence SEQ ID NO: 72 and the polypeptides comprising a sequence having at least 60, 70, 80, 85, 90 or 95% sequence identity with said sequence and exhibiting COMT activity.

According to a preferred embodiment, the recombinant microorganism comprises
a heterologous nucleic acid sequence coding for a 4CL comprising a sequence chosen from the sequences SEQ ID NOs: 6 and 8 and the polypeptides comprising a sequence having at least 60, 70, 80, 85, 90 or 95% sequence identity with one of these sequences and exhibiting 4CL activity; and
a heterologous nucleic acid sequence coding for a CCoA3H comprising a sequence chosen from the sequences SEQ ID NOs: 42, 45 and 49 and the polypeptides comprising a sequence having at least 60, 70, 80, 85, 90 or 95% sequence identity with one of these sequences and exhibiting CCoA3H activity, and preferably a CCoA3H comprising a sequence chosen from the sequence SEQ ID NO: 49 and the polypeptides comprising a sequence having at least 60, 70, 80, 85, 90 or 95% sequence identity with said sequence and exhibiting CCoA3H activity; and
a heterologous nucleic acid sequence coding for an acyl-coenzyme A thioesterase comprising a sequence chosen from the sequences SEQ ID NOs: 2 and 39 and the polypeptides comprising a sequence having at least 60, 70, 80, 85, 90 or 95% sequence identity with one of these sequences and exhibiting acyl-coenzyme A thioesterase activity; and
a heterologous nucleic acid sequence coding for a TAL comprising a sequence chosen from the sequences SEQ ID NOs: 19 and 68, preferably SEQ ID NO: 19, and the polypeptides comprising a sequence having at least 60, 70, 80, 85, 90 or 95% sequence identity with one of these sequences, preferably with SEQ ID NO: 19, and exhibiting TAL activity. Optionally, in this embodiment, the microorganism may also comprise
a heterologous nucleic acid sequence coding for a CCoAMT comprising a sequence chosen from the sequence SEQ ID NO: 40 and the polypeptides comprising a sequence having at least 60, 70, 80, 85, 90 or 95% sequence identity with said sequence and exhibiting CCoAMT activity; and/or
a heterologous nucleic acid sequence coding for a COMT comprising a sequence chosen from the sequences SEQ ID NOs: 72, 74, 76, 79, 80 and 82, and the polypeptides comprising a sequence having at least 60, 70, 80, 85, 90 or 95% sequence identity with one of these sequences and exhibiting COMT activity, preferably comprising a sequence chosen from the sequence SEQ ID NO: 72 and the polypeptides comprising a sequence having at least 60, 70, 80, 85, 90 or 95% sequence identity with said sequence and exhibiting COMT activity.
   Production of p-Coumaric Acid from Phenylalanine

The production of p-coumaric acid from phenylalanine involves enzymes specific to this pathway, namely a phenylalanine ammonia lyase (PAL) capable of producing cinnamic acid from phenylalanine and a cinnamate 4-hydroxylase (C4H) capable of producing p-coumaric acid from cinnamic acid (cf. FIG. 6).

Thus, the microorganism according to the invention may comprise, in addition to the heterologous nucleic acid sequences described above and coding for a 4CL, a CCoA3H, an acyl-coenzyme A thioesterase and optionally for a COMT and/or a CCoAMT (optionally in combination with a CCR and an ALDH) and/or a TAL, a heterologous nucleic acid sequence coding for a phenylalanine ammonia lyase (PAL) and/or a heterologous nucleic acid sequence coding for a cinnamate 4-hydroxylase (C4H), preferably a heterologous nucleic acid sequence coding for a phenylalanine ammonia lyase (PAL) and a heterologous nucleic acid sequence coding for a cinnamate 4-hydroxylase (C4H).

As used herein, the term “phenylalanine ammonia lyase” or “PAL” refers to an enzyme which catalyzes the production of cinnamic acid (also referred to as trans-cinnamic acid) from phenylalanine (EC 4.3.1.24).

The detection of a phenylalanine ammonia lyase activity may be achieved by any method known to those skilled in the art, in vivo or in vitro. The PAL activity may in particular be detected via an enzymatic test consisting of the in vitro incubation of a mixture composed of the enzyme to be tested and phenylalanine under optimal conditions (pH, temperature, ions, etc.). After a certain incubation time, the appearance of cinnamic acid is observed in UPLC-MS in comparison with the expected standard.

Certain PALs may also exhibit a TAL activity and/or a DAL activity.

Several PAL enzymes have already been described in the prior art. Preferably, the PAL originates from a plant, for example a plant of the genus Arabidopsis, Agastache, Ananas, Asparagus, Brassica, Bromheadia, Bambusa, Beta, Betula, Citrus, Cucumis, Camellia, Capsicum, Cassia, Catharanthus, Cicer, Citrullus, Coffea, Cucurbita, Cynodon, Daucus, Dendrobium, Dianthus, Digitalis, Dioscorea, Eucalyptus, Gallus, Ginkgo, Glycine, Hordeum, Helianthus, Ipomoea, Lactuca, Lithospermum, Lotus, Lycopersicon, Medicago, Malus, Manihot, Medicago, Mesembryanthemum, Nicotiana, Olea, Oryza, Phaseolus, Pinus, Populus, Pisum, Persea, Petroselinum, Phalaenopsis, Phyllostachys, Physcomitrella, Picea, Pyrus, Prunus, Quercus, Raphanus, Rehmannia, Rubus, Solanum, Sorghum, Sphenostylis, Stellaria, Stylosanthes, Triticum, Trifolium, Vaccinium, Vigna, Vitis, Zea, or Zinnia. In particular, the PAL may be an enzyme of Arabidopsis thaliana, preferably the enzyme described in SEQ ID NO: 20 or 69, or an enzyme of Citrus sinensis, preferably one of the enzymes described in SEQ ID NOs: 31 and 32.

In a particular embodiment, the PAL comprises a sequence chosen from SEQ ID NOs: 20, 69, 31 and 32 and the polypeptides comprising a sequence having at least 60, 70, 80, 85, 90 or 95% sequence identity with one of these sequences and exhibiting PAL activity. In a preferred embodiment, the PAL comprises a sequence chosen from the sequences SEQ ID NOs: 20, 32 and 69 and the polypeptides comprising a sequence having at least 60, 70, 80, 85, 90 or 95% sequence identity with one of these sequences and exhibiting PAL activity. In a further preferred embodiment, the PAL comprises a sequence chosen from the sequence SEQ ID NO: 20 and the polypeptides comprising a sequence having at least 60, 70, 80, 85, 90 or 95% sequence identity with the sequence SEQ ID NO: 20 and exhibiting PAL activity.

As used herein, the term “cinnamate 4-hydroxylase” or “C4H” refers to an enzyme which catalyzes the production of p-coumaric acid from cinnamic acid (EC 1.14.13.11). This enzyme is CPR-dependent.

The detection of a cinnamate 4-hydroxylase activity may be achieved by any method known to those skilled in the art, in vivo or in vitro. The C4H activity may in particular be detected via an enzymatic test consisting of the in vitro incubation of a mixture composed of the enzyme to be tested, cinnamic acid, NADPH, H+ and 02 under optimal conditions (pH, temperature, ions, etc.). After a certain incubation time, the appearance of p-coumaric acid is observed in UPLC-MS in comparison with the expected standard.

Several C4H enzymes have already been described in the prior art. Preferably, the C4H originates from a plant, for example a plant of the genus Arabidopsis, Ammi, Avicennia, Camellia, Camptotheca, Catharanthus, Citrus, Glycine, Helianthus, Lotus, Mesembryanthemuum, Panicum, Physcomitreila, Phaseolus, Pinus, Populus, Ruta, Saccharum, Solanum, Vitis, Vigna or Zea.

In particular, the C4H may be an enzyme of Arabidopsis thaliana, preferably the enzyme described in SEQ ID NO: 21, an enzyme of Citrus sinensis, preferably one of the enzymes described in SEQ ID NOs: 33 and 34 or an enzyme of Panicum virgatum, preferably the enzyme described in SEQ ID NO: 70.

In a particular embodiment, the C4H comprises a sequence chosen from SEQ ID NOs: 21, 33, 34 and 70 and the polypeptides comprising a sequence having at least 60, 70, 80, 85, 90 or 95% sequence identity with one of these sequences and exhibiting C4H activity. In a preferred embodiment, the C4H comprises a sequence chosen from the sequences SEQ ID NOs: 21 and 70 and the polypeptides comprising a sequence having at least 60, 70, 80, 85, 90 or 95% sequence identity with one of these sequences and exhibiting C4H activity. In a further preferred embodiment, the C4H comprises a sequence chosen from the sequence SEQ ID NO: 21 and the polypeptides comprising a sequence having at least 60, 70, 80, 85, 90 or 95% sequence identity with the sequence SEQ ID NO: 21 and exhibiting C4H activity.

According to one embodiment, the microorganism according to the invention comprises
a heterologous nucleic acid sequence coding for a phenylalanine ammonia lyase (PAL), preferably comprising a sequence chosen from SEQ ID NOs: 20, 69, 31 and 32 and the polypeptides comprising a sequence having at least 60, 70, 80, 85, 90 or 95% sequence identity with one of these sequences and exhibiting PAL activity, and more particularly preferably comprising a sequence chosen from the sequences SEQ ID NOs: 20, 32 and 69, preferably SEQ ID NO: 20, and the polypeptides comprising a sequence having at least 60, 70, 80, 85, 90 or 95% sequence identity with one of these sequences, preferably with SEQ ID NO: 20, and exhibiting PAL activity; and
a heterologous nucleic acid sequence coding for a cinnamate 4-hydroxylase (C4H), preferably comprising a sequence chosen from SEQ ID NOs: 21, 70, 33 and 34 and the polypeptides comprising a sequence having at least 60, 70, 80, 85, 90 or 95% sequence identity with one of these sequences and exhibiting C4H activity, and more particularly preferably comprising a sequence chosen from the sequences SEQ ID NOs: 21 and 70, preferably SEQ ID NO: 21, and the polypeptides comprising a sequence having at least 60, 70, 80, 85, 90 or 95% sequence identity with one of these sequences, preferably with SEQ ID NO: 21, and exhibiting C4H activity.

According to a particular embodiment, the recombinant microorganism according to the invention comprises:
a heterologous nucleic acid sequence coding for a 4CL comprising a sequence chosen from the sequences SEQ ID NOs: 5 to 9 and the polypeptides comprising a sequence having at least 60, 70, 80, 85, 90 or 95% sequence identity with one of these sequences and exhibiting 4CL activity; preferably a 4CL comprising a sequence chosen from the sequences SEQ ID NOs: 5, 6, 8 and 9 and the polypeptides comprising a sequence having at least 60, 70, 80, 85, 90 or 95% sequence identity with one of these sequences and exhibiting 4CL activity, and more particularly preferably a 4CL comprising a sequence chosen from the sequences SEQ ID NOs: 6 and 8 and the polypeptides comprising a sequence having at least 60, 70, 80, 85, 90 or 95% sequence identity with one of these sequences and exhibiting 4CL activity; and
a heterologous nucleic acid sequence coding for a CCoA3H comprising a sequence chosen from the sequences SEQ ID NOs: 42 to 67 and the polypeptides comprising a sequence having at least 60, 70, 80, 85, 90 or 95% sequence identity with one of these sequences and exhibiting CCoA3H activity; preferably a CCoA3H comprising a sequence chosen from the sequences SEQ ID NOs: 42, 45 and 49 and the polypeptides comprising a sequence having at least 60, 70, 80, 85, 90 or 95% sequence identity with one of these sequences and exhibiting CCoA3H activity, and more particularly preferably a CCoA3H comprising a sequence chosen from the sequence SEQ ID NO: 49 and the polypeptides comprising a sequence having at least 60, 70, 80, 85, 90 or 95% sequence identity with said sequence and exhibiting CCoA3H activity; and
a heterologous nucleic acid sequence coding for an acyl-coenzyme A thioesterase comprising a sequence chosen from the sequences SEQ ID NOs: 1, 2 and 39, and the polypeptides comprising a sequence having at least 60, 70, 80, 85, 90 or 95% sequence identity with one of these sequences and exhibiting acyl-coenzyme A thioesterase activity, preferably an acyl-coenzyme A thioesterase comprising a sequence chosen from the sequences SEQ ID NOs: 2 and 39 and the polypeptides comprising a sequence having at least 60, 70, 80, 85, 90 or 95% sequence identity with one of these sequences and exhibiting acyl-coenzyme A thioesterase activity; and
a heterologous nucleic acid sequence coding for a phenylalanine ammonia lyase (PAL), comprising a sequence chosen from SEQ ID NOs: 20, 69, 31 and 32 and the polypeptides comprising a sequence having at least 60, 70, 80, 85, 90 or 95% sequence identity with one of these sequences and exhibiting PAL activity, and more particularly preferably comprising a sequence chosen from the sequences SEQ ID NOs: 20, 32 and 69, preferably SEQ ID NO: 20, and the polypeptides comprising a sequence having at least 60, 70, 80, 85, 90 or 95% sequence identity with one of these sequences, preferably with SEQ ID NO: 20, and exhibiting PAL activity; and
a heterologous nucleic acid sequence coding for a cinnamate 4-hydroxylase (C4H), comprising a sequence chosen from SEQ ID NOs: 21, 70, 33 and 34 and the polypeptides comprising a sequence having at least 60, 70, 80, 85, 90 or 95% sequence identity with one of these sequences and exhibiting C4H activity, and more particularly preferably comprising a sequence chosen from the sequences SEQ ID NOs: 21 and 70, preferably SEQ ID NO: 21, and the polypeptides comprising a sequence having at least 60, 70, 80, 85, 90 or 95% sequence identity with one of these sequences, preferably with SEQ ID NO: 21, and exhibiting C4H activity.

Optionally, in this embodiment, the microorganism may also comprise
a heterologous nucleic acid sequence coding for a CCoAMT comprising a sequence chosen from the sequences SEQ ID NOs: 10 to 16, 40 and 41 and the polypeptides comprising a sequence having at least 60, 70, 80, 85, 90 or 95% sequence identity with one of these sequences and exhibiting CCoAMT activity, preferably a CCoAMT comprising a sequence chosen from the sequence SEQ ID NO: 40 and the polypeptides comprising a sequence having at least 60, 70, 80, 85, 90 or 95% sequence identity with said sequence and exhibiting CCoAMT activity; and/or
a heterologous nucleic acid sequence coding for a COMT comprising a sequence chosen from the sequences SEQ ID NOs: 71 to 92, preferably SEQ ID NOs: 71 to 81, and the polypeptides comprising a sequence having at least 60, 70, 80, 85, 90 or 95% sequence identity with one of these sequences, preferably with one of the sequences SEQ ID NOs: 71 to 81, and exhibiting COMT activity, preferably a COMT comprising a sequence chosen from the sequences SEQ ID NOs: 72 to 74, 76 and 79 to 92, preferably the sequences SEQ ID NOs: 72, 74, 76, 79, 80 and 82, and the polypeptides comprising a sequence having at least 60, 70, 80, 85, 90 or 95% sequence identity with one of these sequences, preferably with one of the sequences SEQ ID NOs: 72, 74, 76, 79, 80 and 82, and exhibiting COMT activity, and more particularly preferably a COMT comprising a sequence chosen from the sequence SEQ ID NO: 72 and the polypeptides comprising a sequence having at least 60, 70, 80, 85, 90 or 95% sequence identity with said sequence and exhibiting COMT activity; and/or
a heterologous nucleic acid sequence coding for a TAL comprising a sequence chosen from the sequences SEQ ID NOs: 19, 30 and 68 and the polypeptides comprising a sequence having at least 60, 70, 80, 85, 90 or 95% sequence identity with one of these sequences and exhibiting TAL activity, preferably a TAL comprising a sequence chosen from the sequences SEQ ID NOs: 19 and 68, preferably SEQ ID NO: 19, and the polypeptides comprising a sequence having at least 60, 70, 80, 85, 90 or 95% sequence identity with one of these sequences, preferably with the sequence SEQ ID NO: 19, and exhibiting TAL activity, preferably, (i) a heterologous nucleic acid sequence coding for a CCoAMT and/or a heterologous nucleic acid sequence coding for a COMT and (ii) a heterologous nucleic acid sequence coding for a TAL.

According to a preferred embodiment, the recombinant microorganism comprises
a heterologous nucleic acid sequence coding for a 4CL comprising a sequence chosen from the sequences SEQ ID NOs: 6 and 8 and the polypeptides comprising a sequence having at least 60, 70, 80, 85, 90 or 95% sequence identity with one of these sequences and exhibiting 4CL activity; and
a heterologous nucleic acid sequence coding for a CCoA3H comprising a sequence chosen from the sequences SEQ ID NOs: 42, 45 and 49 and the polypeptides comprising a sequence having at least 60, 70, 80, 85, 90 or 95% sequence identity with one of these sequences and exhibiting CCoA3H activity, and preferably a CCoA3H comprising a sequence chosen from the sequence SEQ ID NO: 49 and the polypeptides comprising a sequence having at least 60, 70, 80, 85, 90 or 95% sequence identity with said sequence and exhibiting CCoA3H activity; and
a heterologous nucleic acid sequence coding for an acyl-coenzyme A thioesterase comprising a sequence chosen from the sequences SEQ ID NOs: 2 and 39 and the polypeptides comprising a sequence having at least 60, 70, 80, 85, 90 or 95% sequence identity with one of these sequences and exhibiting acyl-coenzyme A thioesterase activity; and
a heterologous nucleic acid sequence coding for a phenylalanine ammonia lyase (PAL) comprising a sequence chosen from the sequences SEQ ID NOs: 20, 32 and 69, preferably SEQ ID NO: 20, and the polypeptides comprising a sequence having at least 60, 70, 80, 85, 90 or 95% sequence identity with one of these sequences, preferably with SEQ ID NO: 20, and exhibiting PAL activity; and
a heterologous nucleic acid sequence coding for a cinnamate 4-hydroxylase (C4H) comprising a sequence chosen from the sequences SEQ ID NOs: 21 and 70, preferably SEQ ID NO: 21, and the polypeptides comprising a sequence having at least 60, 70, 80, 85, 90 or 95% sequence identity with one of these sequences, preferably with SEQ ID NO: 21, and exhibiting C4H activity.

Optionally, in this embodiment, the microorganism may also comprise
a heterologous nucleic acid sequence coding for a CCoAMT comprising a sequence chosen from the sequence SEQ ID NO: 40 and the polypeptides comprising a sequence having at least 60, 70, 80, 85, 90 or 95% sequence identity with said sequence and exhibiting CCoAMT activity; and/or
a heterologous nucleic acid sequence coding for a COMT comprising a sequence chosen from the sequences SEQ ID NOs: 72, 74, 76, 79, 80 and 82, and the polypeptides comprising a sequence having at least 60, 70, 80, 85, 90 or 95% sequence identity with one of these sequences and exhibiting COMT activity, preferably comprising a sequence chosen from the sequence SEQ ID NO: 72 and the polypeptides comprising a sequence having at least 60, 70, 80, 85, 90 or 95% sequence identity with said sequence and exhibiting COMT activity; and/or
a heterologous nucleic acid sequence coding for a TAL comprising a sequence chosen from the sequences SEQ ID NOs: 19 and 68, preferably SEQ ID NO: 19, and the polypeptides comprising a sequence having at least 60, 70, 80, 85, 90 or 95% sequence identity with one of these sequences, preferably with SEQ ID NO: 19, and exhibiting TAL activity, preferably, (i) a heterologous nucleic acid sequence coding for a CCoAMT and/or a heterologous nucleic acid sequence coding for a COMT and (ii) a heterologous nucleic acid sequence coding for a TAL.

### Production of Caffeic Acid Via Non-CoA Pathways

The production of caffeic acid from tyrosine and via pathways involving compounds not coupled to CoA can involve two pathways: the first involves the transformation of tyrosine into p-coumaric acid and then p-coumaric acid into caffeic acid, the second involves the transformation of tyrosine into L-Dopa and then L-Dopa into caffeic acid (cf. FIG. 6). The production of caffeic acid from p-coumaric acid or of L-Dopa from tyrosine may be by an enzyme or enzyme complex with p-coumarate 3-hydroxylase activity.

Thus, the microorganism according to the invention may comprise, in addition to the heterologous nucleic acid sequences described above coding for a 4CL, a CCoA3H, an acyl-coenzyme A thioesterase and optionally for a COMT and/or a CCoAMT (optionally in combination with a CCR and an ALDH) and/or a TAL and/or a PAL and/or a C4H, one or more heterologous nucleic acid sequences coding for an enzyme or enzyme complex having p-coumarate 3-hydroxylase activity.

As used here, the term “p-coumarate 3-hydroxylase activity” refers to an enzyme or enzyme complex that catalyzes the transformation of p-coumaric acid into caffeic acid and/or of L-tyrosine into L-Dopa. To determine if there is p-coumarate 3-hydroxylase activity, an enzymatic test can be carried out which consists in the in vitro incubation of the enzyme or enzyme complex, p-coumaric acid or L-tyrosine, and possibly FAD and NADH, under optimal conditions (pH, temperature, ions, etc.). After a certain incubation time, the appearance of caffeic acid or of L-Dopa is observed in HPLC-MS in comparison with the expected standard. In particular, this activity may be the result of an enzyme complex comprising a 4-hydroxyphenylacetate 3-monooxygenase oxygenase (HpaB) and a 4-hydroxyphenylacetate 3-monooxygenase reductase (HpaC).

The term “4-hydroxyphenylacetate 3-monooxygenase oxygenase” refers to an enzyme exhibiting p-coumarate 3-hydroxylase activity when in the presence of a 4-hydroxyphenylacetate 3-monooxygenase reductase. The term “4-hydroxyphenylacetate 3-monooxygenase reductase” refers to an enzyme exhibiting p-coumarate 3-hydroxylase activity when in the presence of a 4-hydroxyphenylacetate 3-monooxygenase oxygenase. Preferably, the HpaB and HpaC enzymes are produced by bacteria, preferably Escherichia coli, bacteria of the genus Pseudomonas, in particular Pseudomonas aeruginosa, or bacteria of the genus Salmonella, in particular Salmonella enterica. The HpaB and HpaC enzymes can originate from the same bacterium or from different bacteria.

In particular, the HpaB may be an enzyme of Pseudomonas aeruginosa, in particular the HpaB described in SEQ ID NO: 17, or an enzyme of Escherichia coli, in particular the HpaB described in SEQ ID NO: 26.

According to one embodiment, the HpaB comprises a sequence chosen from the sequences SEQ ID NOs: 17 and 26 and polypeptides comprising a sequence having at least 60, 70, 80, 85, 90 or 95% sequence identity with any of these sequences and exhibiting 4-hydroxyphenylacetate 3-monooxygenase oxygenase activity.

According to a preferred embodiment, the HpaB comprises a sequence chosen from the sequence SEQ ID NO: 17 and polypeptides comprising a sequence having at least 60, 70, 80, 85, 90 or 95% sequence identity with sequence SEQ ID NO: 17 and exhibiting 4-hydroxyphenylacetate 3-monooxygenase oxygenase activity.

The HpaC may be an enzyme of Salmonella enterica, in particular the HpaC described in SEQ ID NO: 18, or an enzyme of Escherichia coli, in particular the HpaC described in SEQ ID NO: 27.

According to one embodiment, the HpaC comprises a sequence chosen from the sequences SEQ ID NOs: 18 and 27 and polypeptides comprising a sequence having at least 60, 70, 80, 85, 90 or 95% sequence identity with any of these sequences and exhibiting 4-hydroxyphenylacetate 3-monooxygenase reductase activity.

According to a preferred embodiment, the HpaC comprises a sequence chosen from the sequence SEQ ID NO: 18 and polypeptides comprising a sequence having at least 60, 70, 80, 85, 90 or 95% sequence identity with sequence SEQ ID NO: 18 and exhibiting 4-hydroxyphenylacetate 3-monooxygenase reductase activity.

According to a particular embodiment, the microorganism comprises
a heterologous nucleic acid sequence coding for a 4-hydroxyphenylacetate 3-monooxygenase oxygenase, preferably comprising a sequence chosen from the sequences SEQ ID NOs: 17 and 26 and polypeptides comprising a sequence having at least 60, 70, 80, 85, 90 or 95% sequence identity with any of these sequences and exhibiting 4-hydroxyphenylacetate 3-monooxygenase oxygenase activity, and more particularly preferably a sequence chosen from SEQ ID NOs: 17 and polypeptides comprising a sequence having at least 60, 70, 80, 85, 90 or 95% sequence identity with sequence SEQ ID NO: 17 and exhibiting 4-hydroxyphenylacetate 3-monooxygenase oxygenase activity, and
a heterologous nucleic acid sequence coding for a 4-hydroxyphenylacetate 3-monooxygenase reductase, preferably comprising a sequence chosen from sequences SEQ ID NOs: 18 and 27 and polypeptides comprising a sequence having at least 60, 70, 80, 85, 90 or 95% sequence identity with any of these sequences and exhibiting 4-hydroxyphenylacetate 3-monooxygenase reductase activity, and more particularly preferably a sequence chosen from SEQ ID NOs: 18 and polypeptides comprising a sequence having at least 60, 70, 80, 85, 90 or 95% sequence identity with sequence SEQ ID NO: 18 and exhibiting 4-hydroxyphenylacetate 3-monooxygenase reductase activity.

As an alternative to or in combination with HpaB and HpaC, an enzyme capable of converting tyrosine into L-Dopa, namely a 4-methoxybenzoate O-demethylase, and an enzyme capable of converting p-coumaric acid into caffeic acid, namely a p-coumarate 3-hydroxylase, can be used (cf. FIG. 6). These enzymes are members of the cytochrome P450 (CYP) family and are CPR-dependent, i.e. active in the presence of a cytochrome P450 reductase (CPR). The cytochrome P450 reductase can be an endogenous enzyme or a heterologous enzyme.

Thus, the microorganism according to the invention may comprise a heterologous nucleic acid sequence coding for a CPR-dependent p-coumarate 3-hydroxylase (C3H), i.e. an enzyme capable of converting p-coumaric acid into caffeic acid in the presence of a CPR (EC 1.14.13). The p-coumarate 3-hydroxylase activity of this enzyme can be tested as indicated above and in the presence of a CPR.

The C3H can be a bacterial enzyme, in particular of bacteria of the genus Saccharothrix. In particular, the C3H may be an enzyme of Saccharothrix espanaensis, preferably the enzyme described in SEQ ID NO: 25.

In a particular embodiment, the C3H comprises a sequence chosen from sequence SEQ ID NO: 25 and polypeptides comprising a sequence having at least 60, 70, 80, 85, 90 or 95% sequence identity with sequence SEQ ID NO: 25 and exhibiting p-coumarate 3-hydroxylase activity.

The microorganism according to the invention may also comprise a heterologous nucleic acid sequence coding for a 4-methoxybenzoate O-demethylase, i.e. an enzyme capable of converting L-tyrosine into L-Dopa in the presence of a CPR (EC 1.14.99.15). The p-coumarate 3-hydroxylase activity of this enzyme can be tested as indicated above and in the presence of a CPR and tyrosine.

The 4-methoxybenzoate O-demethylase can be an enzyme from bacteria, in particular Rhodopseudomonas palustris, Pseudomonas putida, or Escherichia coli, plants, in particular Beta vulgaris, mammals, in particular Oryctolagus cuniculus, or fungi, in particular Rhodotorula glutinis. In a particular embodiment, the 4-methoxybenzoate O-demethylase is an enzyme of Rhodopseudomonas palustris, in particular the enzyme described in SEQ ID NO: 28, or of Beta vulgaris, in particular the enzyme described in SEQ ID NO: 29.

In a particular embodiment, the 4-methoxybenzoate O-demethylase comprises a sequence chosen from SEQ ID NOs: 28 and 29 and polypeptides comprising a sequence having at least 60, 70, 80, 85, 90 or 95% sequence identity with one of these sequences and exhibiting L-tyrosine hydrolase activity.

The production of caffeic acid from L-Dopa involves an enzyme exhibiting a dihydroxyphenylalanine ammonia lyase (DAL) activity. According to one embodiment, the microorganism according to the invention may comprise a heterologous nucleic acid sequence coding for an enzyme having a dihydroxyphenylalanine ammonia lyase activity.

As used here, the term “dihydroxyphenylalanine ammonia lyase” or “DAL” refers to an enzyme that catalyzes the production of caffeic acid from L-Dopa (EC 4.3.1.11).

The detection of a dihydroxyphenylalanine ammonia lyase activity can be carried out by any method known to those skilled in the art, in vivo or in vitro. DAL activity can in particular be detected via an enzymatic test consisting in the in vitro incubation of a mixture composed of the enzyme to be tested and L-Dopa under optimal conditions (pH, temperature, ions, etc.). After a certain incubation time, the appearance of caffeic acid is observed in UPLC-MS in comparison with the expected standard.

Some DALs may also exhibit a TAL and/or PAL activity.

According to one embodiment, the microorganism according to the invention comprises
(i)—a heterologous nucleic acid sequence coding for a 4-hydroxyphenylacetate 3-monooxygenase oxygenase, preferably comprising a sequence chosen from sequences SEQ ID NOs: 17 and 26 and polypeptides comprising a sequence having at least 60, 70, 80, 85, 90 or 95% sequence identity with any of these sequences and exhibiting 4-hydroxyphenylacetate 3-monooxygenase oxygenase activity, and more particularly preferably a sequence chosen from SEQ ID NOs: 17 and polypeptides comprising a sequence having at least 60, 70, 80, 85, 90 or 95% sequence identity with sequence SEQ ID NO: 17 and exhibiting 4-hydroxyphenylacetate 3-monooxygenase oxygenase activity, and
a heterologous nucleic acid sequence coding for a 4-hydroxyphenylacetate 3-monooxygenase reductase, preferably comprising a sequence chosen from sequences SEQ ID NOs: 18 and 27 and polypeptides comprising a sequence having at least 60, 70, 80, 85, 90 or 95% sequence identity with any of these sequences and exhibiting 4-hydroxyphenylacetate 3-monooxygenase reductase activity, and more particularly preferably a sequence chosen from SEQ ID NOs: 18 and polypeptides comprising a sequence having at least 60, 70, 80, 85, 90 or 95% sequence identity with sequence SEQ ID NO: 18 and exhibiting a 4-hydroxyphenylacetate 3-monooxygenase reductase activity; and/or
(ii) a heterologous nucleic acid sequence coding for a C3H, preferably comprising a sequence chosen from sequence SEQ ID NO: 25 and polypeptides comprising a sequence having at least 60, 70, 80, 85, 90 or 95% sequence identity with sequence SEQ ID NO: 25 and exhibiting p-coumarate 3-hydroxylase activity; and/or
(iii) a heterologous nucleic acid sequence coding for a 4-methoxybenzoate O-demethylase, preferably comprising a sequence chosen from SEQ ID NOs: 28 and 29 and polypeptides comprising a sequence having at least 60, 70, 80, 85, 90 or 95% sequence identity with one of these sequences and exhibiting L-tyrosine hydrolase activity.

According to a particular embodiment, the recombinant microorganism according to the invention comprises:
a heterologous nucleic acid sequence coding for a 4CL comprising a sequence chosen from sequences SEQ ID NOs: 5 to 9 and polypeptides comprising a sequence having at least 60, 70, 80, 85, 90 or 95% sequence identity with any of these sequences and exhibiting 4CL activity; preferably a 4CL comprising a sequence chosen from sequences SEQ ID NOs: 5, 6, 8 and 9 and polypeptides comprising a sequence having at least 60, 70, 80, 85, 90 or 95% sequence identity with any of these sequences and exhibiting 4CL activity, and more particularly preferably a 4CL comprising a sequence chosen from sequences SEQ ID NOs: 6 and 8 and polypeptides comprising a sequence having at least 60, 70, 80, 85, 90 or 95% sequence identity with any of these sequences and exhibiting 4CL activity; and
a heterologous nucleic acid sequence coding for a CCoA3H comprising a sequence chosen from sequences SEQ ID NOs: 42 to 67 and polypeptides comprising a sequence having at least 60, 70, 80, 85, 90 or 95% sequence identity with any of these sequences and exhibiting CCoA3H activity; preferably a CCoA3H comprising a sequence chosen from sequences SEQ ID NO: 42, 45 and 49 and polypeptides comprising a sequence having at least 60, 70, 80, 85, 90 or 95% sequence identity with any of these sequences and exhibiting CCoA3H activity, and more particularly preferably a CCoA3H comprising a sequence chosen from sequence SEQ ID NO: 49 and polypeptides comprising a sequence having at least 60, 70, 80, 85, 90 or 95% sequence identity with said sequence and exhibiting CCoA3H activity; and
a heterologous nucleic acid sequence coding for an acyl-coenzyme A thioesterase comprising a sequence chosen from sequences SEQ ID NOs: 1, 2 and 39, and polypeptides comprising a sequence having at least 60, 70, 80, 85, 90 or 95% sequence identity with any of these sequences and exhibiting acyl-coenzyme A thioesterase activity, preferably an acyl-coenzyme A thioesterase comprising a sequence chosen from sequences SEQ ID NOs: 2 and 39 and polypeptides comprising a sequence having at least 60, 70, 80, 85, 90 or 95% sequence identity with any of these sequences and exhibiting acyl-coenzyme A thioesterase activity; and
(i)—a heterologous nucleic acid sequence coding for a 4-hydroxyphenylacetate 3-monooxygenase oxygenase, preferably comprising a sequence chosen from sequences SEQ ID NOs: 17 and 26 and polypeptides comprising a sequence having at least 60, 70, 80, 85, 90 or 95% sequence identity with any of these sequences and exhibiting 4-hydroxyphenylacetate 3-monooxygenase oxygenase activity, and more particularly preferably a sequence chosen from SEQ ID NOs: 17 and polypeptides comprising a sequence having at least 60, 70, 80, 85, 90 or 95% sequence identity with sequence SEQ ID NO: 17 and exhibiting 4-hydroxyphenylacetate 3-monooxygenase oxygenase activity, and
a heterologous nucleic acid sequence coding for a 4-hydroxyphenylacetate 3-monooxygenase reductase, preferably comprising a sequence chosen from sequences SEQ ID NOs: 18 and 27 and polypeptides comprising a sequence having at least 60, 70, 80, 85, 90 or 95% sequence identity with any of these sequences and exhibiting 4-hydroxyphenylacetate 3-monooxygenase reductase activity, and more particularly preferably a sequence chosen from SEQ ID NOs: 18 and polypeptides comprising a sequence having at least 60, 70, 80, 85, 90 or 95% sequence identity with sequence SEQ ID NO: 18 and exhibiting a 4-hydroxyphenylacetate 3-monooxygenase reductase activity; and/or
(ii) a heterologous nucleic acid sequence coding for a C3H, preferably comprising a sequence chosen from sequence SEQ ID NO: 25 and polypeptides comprising a sequence having at least 60, 70, 80, 85, 90 or 95% sequence identity with sequence SEQ ID NO: 25 and exhibiting p-coumarate 3-hydroxylase activity; and/or
(iii) a heterologous nucleic acid sequence coding for a 4-methoxybenzoate O-demethylase, preferably comprising a sequence chosen from SEQ ID NOs: 28 and 29 and polypeptides comprising a sequence having at least 60, 70, 80, 85, 90 or 95% sequence identity with one of these sequences and exhibiting L-tyrosine hydrolase activity.

Optionally, in this embodiment, the microorganism may also comprise
a heterologous nucleic acid sequence coding for a TAL comprising a sequence chosen from sequences SEQ ID NOs: 19, 30 and 68 and polypeptides comprising a sequence having at least 60, 70, 80, 85, 90 or 95% sequence identity with one of these sequences and exhibiting TAL activity, preferably a TAL comprising a sequence chosen from sequences SEQ ID NO: 19 and 68, preferably SEQ ID NO: 19, and polypeptides comprising a sequence having at least 60, 70, 80, 85, 90 or 95% sequence identity with any of these sequences, preferably with SEQ ID NO: 19, and exhibiting TAL activity and/or
a heterologous nucleic acid sequence coding for a phenylalanine ammonia lyase (PAL) comprising a sequence chosen from SEQ ID NOs: 20, 69, 31 and 32 and polypeptides comprising a sequence having at least 60, 70, 80, 85, 90 or 95% sequence identity with any of these sequences and exhibiting PAL activity, and more particularly preferably, comprising a sequence chosen from sequences SEQ ID NOs: 20, 32 and 69, preferably SEQ ID NOs: 20 and polypeptides comprising a sequence having at least 60, 70, 80, 85, 90 or 95% sequence identity with one of these sequences, preferably with SEQ ID NO: 20, and exhibiting PAL activity; and/or
a heterologous nucleic acid sequence coding for a cinnamate 4-hydroxylase (C4H) comprising a sequence chosen from SEQ ID NOs: 21, 70, 33 and 34 and polypeptides comprising a sequence having at least 60, 70, 80, 85, 90 or 95% sequence identity with any of these sequences and exhibiting C4H activity, and more particularly preferably, comprising a sequence chosen from sequences SEQ ID NOs: 21 and 70, preferably SEQ ID NO: 21 and polypeptides comprising a sequence having at least 60, 70, 80, 85, 90 or 95% sequence identity with any of these sequences, preferably with SEQ ID NO: 21, and exhibiting C4H activity; and/or
a heterologous nucleic acid sequence coding for a CCoAMT comprising a sequence chosen from sequences SEQ ID NOs: 10 to 16, 40 and 41 and polypeptides comprising a sequence having at least 60, 70, 80, 85, 90 or 95% sequence identity with any of these sequences and exhibiting CCoAMT activity, preferably a CCoAMT comprising a sequence chosen from sequence SEQ ID NO: 40 and polypeptides comprising a sequence having at least 60, 70, 80, 85, 90 or 95% sequence identity with said sequence and exhibiting CCoAMT activity; and/or
a heterologous nucleic acid sequence coding for a COMT comprising a sequence chosen from sequences SEQ ID NOs: 71 to 92, preferably SEQ ID NOs: 71 to 81, and polypeptides comprising a sequence having at least 60, 70, 80, 85, 90 or 95% sequence identity with any of these sequences, preferably with any of sequences SEQ ID NOs: 71 to 81, and exhibiting COMT activity, preferably a COMT comprising a sequence chosen from sequences SEQ ID NOs: 72 to 74, 76 and 79 to 92, preferably the sequences SEQ ID NOs: 72, 74, 76, 79, 80 and 82, and polypeptides comprising a sequence having at least 60, 70, 80, 85, 90 or 95% sequence identity with any of these sequences, preferably with any of sequences SEQ ID NOs: 72, 74, 76, 79, 80 and 82, and exhibiting COMT activity, and more particularly preferably a COMT comprising a sequence chosen from sequence SEQ ID NO: 72 and polypeptides comprising a sequence having at least 60, 70, 80, 85, 90 or 95% sequence identity with said sequence and exhibiting COMT activity, preferably, (i) a heterologous nucleic acid sequence coding for a CCoAMT and/or a heterologous nucleic acid sequence coding for a COMT and (ii) a heterologous nucleic acid sequence coding for a TAL, a heterologous nucleic acid sequence coding for a cinnamate 4-hydroxylase (C4H) and a heterologous nucleic acid sequence coding for a phenylalanine ammonia lyase (PAL).

According to a preferred embodiment, the recombinant microorganism according to the invention comprises:
a heterologous nucleic acid sequence coding for a 4CL comprising a sequence chosen from sequences SEQ ID NOs: 6 and 8 and polypeptides comprising a sequence having at least 60, 70, 80, 85, 90 or 95% sequence identity with any of these sequences and exhibiting 4CL activity; and
a heterologous nucleic acid sequence coding for a CCoA3H comprising a sequence chosen from sequences SEQ ID NOs: 42, 45 and 49 and polypeptides comprising a sequence having at least 60, 70, 80, 85, 90 or 95% sequence identity with any of these sequences and exhibiting CCoA3H activity, and preferably a CCoA3H comprising a sequence chosen from sequence SEQ ID NO: 49 and polypeptides comprising a sequence having at least 60, 70, 80, 85, 90 or 95% sequence identity with said sequence and exhibiting CCoA3H activity; and
a heterologous nucleic acid sequence coding for an acyl-coenzyme A thioesterase comprising a sequence chosen from sequences SEQ ID NOs: 2 and 39 and polypeptides comprising a sequence having at least 60, 70, 80, 85, 90 or 95% sequence identity with any of these sequences and exhibiting acyl-coenzyme A thioesterase activity; and
a heterologous nucleic acid sequence coding for a 4-hydroxyphenylacetate 3-monooxygenase oxygenase comprising a sequence chosen from SEQ ID NO: 17 and polypeptides comprising a sequence having at least 60, 70, 80, 85, 90 or 95% sequence identity with sequence SEQ ID NO: 17 and exhibiting a 4-hydroxyphenylacetate 3-monooxygenase oxygenase activity, and
a heterologous nucleic acid sequence coding for a 4-hydroxyphenylacetate 3-monooxygenase reductase comprising a sequence chosen from SEQ ID NO: 18 and polypeptides comprising a sequence having at least 60, 70, 80, 85, 90 or 95% sequence identity with sequence SEQ ID NO: 18 and exhibiting a 4-hydroxyphenylacetate 3-monooxygenase reductase activity.

Optionally, in this embodiment, the microorganism may also comprise
a heterologous nucleic acid sequence coding for a TAL comprising a sequence chosen from sequences SEQ ID NOs: 19 and 68, preferably SEQ ID NO: 19, and polypeptides comprising a sequence having at least 60, 70, 80, 85, 90 or 95% sequence identity with any of these sequences, preferably with SEQ ID NO: 19, and exhibiting TAL activity and/or
a heterologous nucleic acid sequence coding for a phenylalanine ammonia lyase (PAL) comprising a sequence chosen from sequences SEQ ID NOs: 20, 32 and 69, preferably SEQ ID NO: 20, and polypeptides comprising a sequence having at least 60, 70, 80, 85, 90 or 95% sequence identity with any of these sequences, preferably with SEQ ID NO: 20, and exhibiting PAL activity; and/or
a heterologous nucleic acid sequence coding for a cinnamate 4-hydroxylase (C4H) comprising a sequence chosen from sequences SEQ ID NOs: 21 and 70, preferably SEQ ID NO: 21, and polypeptides comprising a sequence having at least 60, 70, 80, 85, 90 or 95% sequence identity with any of these sequences, preferably with SEQ ID NO: 21, and exhibiting C4H activity; and/or
a heterologous nucleic acid sequence coding for a CCoAMT comprising a sequence chosen from sequence SEQ ID NO: 40 and polypeptides comprising a sequence having at least 60, 70, 80, 85, 90 or 95% sequence identity with said sequence and exhibiting CCoAMT activity; and/or
a heterologous nucleic acid sequence coding for a COMT comprising a sequence chosen from sequences SEQ ID NOs: 72, 74, 76, 79, 80 and 82, and polypeptides comprising a sequence having at least 60, 70, 80, 85, 90 or 95% sequence identity with any of these sequences and exhibiting COMT activity, preferably comprising a sequence chosen from sequence SEQ ID NO: 72 and polypeptides comprising a sequence having at least 60, 70, 80, 85, 90 or 95% sequence identity with said sequence and exhibiting COMT activity, preferably, (i) a heterologous nucleic acid sequence coding for a CCoAMT and/or a heterologous nucleic acid sequence coding for a COMT and (ii) a heterologous nucleic acid sequence coding for a TAL, a heterologous nucleic acid sequence coding for a cinnamate 4-hydroxylase (C4H) and a heterologous nucleic acid sequence coding for a phenylalanine ammonia lyase (PAL).

### CPR: Cytochrome P450 Reductase

Some enzymes mentioned above, such as C3H or C4H, are CPR-dependent enzymes. The activities of these enzymes require the presence of NADPH and thus the presence of a cytochrome P450 reductase (CPR), in particular an NADPH-cytochrome P450 reductase.

Thus, in the various embodiments described above and below relating to the microorganism according to the invention, the latter may also comprise an endogenous nucleic acid coding for a cytochrome P450 reductase. Optionally, the endogenous CPR may be overexpressed, for example by replacing the promoter of the endogenous gene with a strong heterologous promoter and/or by increasing the number of copies of the endogenous gene.

Alternatively, or in addition to this endogenous nucleic acid, the microorganism according to the invention may comprise a heterologous nucleic acid sequence which codes for a cytochrome P450 reductase (CPR).

As used here, the term “cytochrome P450 reductase” or “CPR” refers to an enzyme involved in electron transfer from NADPH and belonging to class EC 1.6.2.4.

The detection of a CPR activity can be carried out by any method known to those skilled in the art, in vivo or in vitro. CPRs are enzymes that catalyze the transfer of electrons from NADPH to cytochromes p450 (for example C3H or C4H). Thus, CPR activity can in particular be detected using an enzymatic kit combining the oxidation of NADPH by CPR with the reduction of a colorless substrate into a colored product with an absorbance peak at a given wavelength, the rate of color generation being directly proportional to the CPR activity. An example of a commercial kit based on this principle is the “CPR activity assay kit” from PromoKine (Cat #PK-CA577-K700).

The CPR preferably originates from a eukaryote, in particular a yeast, for example a yeast of the genus Saccharomyces, or a plant, for example a plant of the genus Arabidopsis, Ammi, Avicennia, Camellia, Camptotheca, Catharanthus, Citrus, Glycine, Helianthus, Lotus, Mesembryanthemum, Phaseolus, Physcomitrella, Pinus, Populus, Ruta, Saccharum, Solanum, Vigna, Vitis or Zea.

In particular, the CPR may be an enzyme of Catharanthus roseus, preferably the enzyme described in SEQ ID NO: 22, an enzyme of Saccharomyces cerevisiae, preferably the enzyme described in SEQ ID NO: 35, or an enzyme of Arabidopsis thaliana, preferably one of the enzymes described in SEQ ID NOs: 36 and 37. The CPR can also be a chimeric protein such as that described in the article by Aigrain et al (2009, EMBO reports, 10, 742-747; SEQ ID NO: 38).

In a particular embodiment, the CPR comprises a sequence chosen from SEQ ID NOs: 22, 35, 36, 37 and 38 and polypeptides comprising a sequence having at least 60, 70, 80, 85, 90 or 95% sequence identity with any of these sequences and exhibiting CPR activity.

In a preferred embodiment, the CPR comprises a sequence chosen from SEQ ID NO: 22 and polypeptides comprising a sequence having at least 60, 70, 80, 85, 90 or 95% sequence identity with sequence SEQ ID NO: 22 and exhibiting CPR activity.

Increased Production of Tyrosine and/or Phenylalanine

In the various embodiments described above and below concerning the microorganism according to the invention, the latter can also be modified to increase the production of tyrosine and/or phenylalanine, preferably tyrosine. Thus, in preferred embodiments, the microorganism according to the invention produces large amounts of tyrosine and/or phenylalanine, in particular from a simple carbon source such as glucose.

This increase may be obtained by any method known to those skilled in the art and in particular by expressing one or more variants of one or more enzymes involved in the synthesis of these amino acids, said variants being resistant to feedback by tyrosine and/or phenylalanine, preferably resistant to tyrosine feedback.

In particular, the microorganism can be modified to express a variant of 3-deoxy-D-arabino-hepturosonate-7-phosphate (DAHP) synthase (EC 2.5.1.54) and/or of chorismate mutase (EC 5.4.99.5), resistant to tyrosine feedback. Such variants are well known to those skilled in the art (see, for example, Gold et al., Microb Cell Fact. 2015; 14: 73).

Thus, according to a particular embodiment, the microorganism according to the invention comprises a heterologous nucleic acid sequence coding for a 3-deoxy-D-arabino-hepturosonate-7-phosphate (DAHP) synthase resistant to tyrosine feedback and/or a heterologous nucleic acid sequence coding for a chorismate mutase resistant to tyrosine feedback.

In the yeast S. cerevisiae, 3-deoxy-D-arabino-hepturosonate-7-phosphate (DAHP) synthase is encoded by the ARO4 gene (NCBI Gene ID: 852551) and chorismate mutase is encoded by the ARO7 gene (NCBI Gene ID: 856173). Variants resistant to tyrosine feedback of these enzymes are known, for example the mutant ARO4^{K229L} (SEQ ID NO: 23) and the mutant ARO7^{G141S} (SEQ ID NO: 24).

Thus, according to a preferred embodiment, the microorganism according to the invention comprises
a heterologous nucleic acid sequence coding for a tyrosine feedback-resistant 3-deoxy-D-arabino-hepturosonate-7-phosphate (DAHP) synthase comprising a sequence chosen from SEQ ID NO: 23 and polypeptides comprising a sequence having at least 60, 70, 80, 85, 90 or 95% sequence identity with sequence SEQ ID NO: 23, said polypeptides exhibiting a tyrosine feedback-resistant 3-deoxy-D-arabino-hepturosonate-7-phosphate (DAHP) synthase activity and preferably a leucine at the position corresponding to position 229 of SEQ ID NO: 23; and/or
a heterologous nucleic acid sequence coding for a chorismate mutase resistant to tyrosine feedback and comprising a sequence chosen from SEQ ID NO: 24 and polypeptides comprising a sequence having at least 60, 70, 80, 85, 90 or 95% sequence identity with sequence SEQ ID NO: 24, said polypeptides exhibiting a tyrosine feedback-resistant chorismate mutase activity and preferably a serine at the position corresponding to position 141 of SEQ ID NO: 24.

Alternatively or cumulatively, increased tyrosine and/or phenylalanine production can also be achieved by redirecting the flow of carbon from other metabolic pathways to that of tyrosine and/or phenylalanine, preferably tyrosine. These modifications and the genes involved are well known to those skilled in the art (see U.S. Pat. No. 8,809,028; Pandey et al., 2016, Biotechnol Adv., 34, 634-662).

Thus, according to a particular embodiment, in the microorganism according to the invention, one or more endogenous genes coding for an enzyme involved in the Ehrlich amino acid degradation pathway are inactivated. The gene or genes may be inactivated by any method known to those skilled in the art, in particular by total or partial deletion, or by insertion of a nucleic sequence into the coding sequence, in particular a sequence shifting the reading frame or inserting a stop codon.

In particular, in the microorganism according to the invention, an endogenous gene coding for a phenylpyruvate decarboxylase can be inactivated. This enzyme is responsible for the first step in the Ehrlich amino acid degradation pathway. The phenylpyruvate decarboxylase in the yeast S. cerevisiae is encoded by the ARO10 gene (NCBI Gene ID: 851987).

### Inactivation of Ferulic Acid Decarboxylase

In the various embodiments described above relating to the microorganism according to the invention, the latter can also be modified to inactivate the endogenous gene or genes coding for a ferulic acid decarboxylase.

This enzyme catalyzes in particular the decarboxylation of ferulic acid, p-coumaric acid and cinnamic acid to produce their vinyl derivatives, namely 4-vinylguaiacol, 4-vinylphenol and styrene, respectively. It belongs to class EC 4.1.1.102. Its inactivation increases the available amounts of cinnamic acid and p-coumaric acid and the amounts of ferulic acid produced in the microorganism according to the invention.

The gene or genes coding for this enzyme in the microorganism according to the invention can be easily identified by a person skilled in the art. For example, in the yeast Saccharomyces cerevisiae, ferulic acid decarboxylase is encoded by the FDC1 gene (NCBI Gene ID: 852152).

The gene or genes may be inactivated by any method known to those skilled in the art, in particular by total or partial deletion, or by insertion of a nucleic sequence into the coding sequence, in particular a sequence shifting the reading frame or inserting a stop codon.

### Recombinant Microorganism Comprising a Heterologous Nucleic Acid Sequence Coding for a Caffeic Acid O-Methyltransferase (COMT)

The inventors have identified various COMT enzymes that are particularly effective in producing ferulic acid from caffeic acid. These enzymes thus significantly improve ferulic acid production obtained from recombinant microorganisms, whether or not they use a pathway for the synthesis of caffeic acid involving compounds linked to coenzyme A (CoA). Thus, according to another aspect, the present invention relates to a recombinant microorganism comprising a heterologous nucleic acid sequence coding for a caffeic acid O-methyltransferase (COMT).

COMT is an enzyme of a plant of the genus Panicum, Catharanthus, Triticum, Nicotiana, Picea, Zea, Saccharum, Stylosanthes, Cucumis, Tarenaya, Ziziphus, Cucurbita, Ipomoea, Thalictrum, Punica, Brassica, Lycium or Acer, preferably of the genus Panicum, Catharanthus, Triticum, Zea, Saccharum, Stylosanthes, Cucumis, Tarenaya, Ziziphus, Cucurbita, Ipomoea, Thalictrum, Punica, Brassica, Lycium or Acer, and more particularly preferably of the genus Panicum, Triticum, Zea or Stylosanthes.

In particular, the COMT may be an enzyme of Panicum virgatum, Catharanthus roseus, Triticum aestivum, Nicotiana tabacum, Picea abies, Zea mays, Stylosanthes humilis, Saccharum officinarum, Cucumis sativus, Tarenaya hassleriana, Ziziphus jujuba var. spinosa, Cucurbita maxima, Ipomoea nil, Thalictrum tuberosum, Punica granatum, Brassica cretica, Lycium chinense or Acer yangbiense, preferably an enzyme of Panicum virgatum, Catharanthus roseus, Triticum aestivum, Zea mays, Stylosanthes humilis, Saccharum officinarum, Cucumis sativus, Tarenaya hassleriana, Ziziphus jujuba var. spinosa, Cucurbita maxima, Ipomoea nil, Thalictrum tuberosum, Punica granatum, Brassica cretica, Lycium chinense or Acer yangbiense, and more particularly preferably an enzyme of Panicum virgatum, Triticum aestivum, Zea mays or Stylosanthes humilis.

In particular, the COMT may be an enzyme of Panicum virgatum, in particular the COMT described in SEQ ID NO: 71 or 76, an enzyme of Catharanthus roseus, in particular the COMT described in SEQ ID NO: 73, an enzyme of Triticum aestivum, in particular the COMT described in SEQ ID NO: 74 or 75, an enzyme of Nicotiana tabacum, in particular the COMT described in SEQ ID NO: 77, an enzyme of Picea abies, in particular the COMT described in SEQ ID NO: 78, an enzyme of Zea mays, in particular the COMT described in SEQ ID NO: 79 or SEQ ID NO: 82, an enzyme of Stylosanthes humilis, in particular the COMT described in SEQ ID NO: 80, an enzyme of Saccharum officinarum, in particular the COMT described in SEQ ID NO: 81, an enzyme of Cucumis sativus, in particular the COMT described in SEQ ID NO: 83, an enzyme of Tarenaya hassleriana, in particular the COMT described in SEQ ID NO: 84, an enzyme of Ziziphus jujuba var. spinosa, in particular the COMT described in SEQ ID NO: 85, an enzyme of Cucurbita maxima, in particular the COMT described in SEQ ID NO: 86, an enzyme of Ipomoea nil, in particular the COMT described in SEQ ID NO: 87, an enzyme of Thalictrum tuberosum, in particular the COMT described in SEQ ID NO: 88, an enzyme of Punica granatum, in particular the COMT described in SEQ ID NO: 89, an enzyme of Brassica cretica, in particular the COMT described in SEQ ID NO: 90, an enzyme of Lycium chinense, in particular the COMT described in SEQ ID NO: 91, or an enzyme of Acer yangbiense, in particular the COMT described in SEQ ID NO: 92.

More particularly, the COMT may be an enzyme of Panicum virgatum, in particular the COMT described in SEQ ID NO: 71 or 76, an enzyme of Catharanthus roseus, in particular the COMT described in SEQ ID NO: 73, an enzyme of Triticum aestivum, in particular the COMT described in SEQ ID NO: 74, an enzyme of Zea mays, in particular the COMT described in SEQ ID NO: 79 or SEQ ID NO: 82, an enzyme of Stylosanthes humilis, in particular the COMT described in SEQ ID NO: 80, an enzyme of Saccharum officinarum, in particularthe COMT described in SEQ ID NO: 81, an enzyme of Cucumissativus, in particular the COMT described in SEQ ID NO: 83, an enzyme of Tarenaya hassleriana, in particular the COMT described in SEQ ID NO: 84, an enzyme of Ziziphus jujuba var. spinosa, in particular the COMT described in SEQ ID NO: 85, an enzyme of Cucurbita maxima, in particular the COMT described in SEQ ID NO: 86, an enzyme of Ipomoea nil, in particular the COMT described in SEQ ID NO: 87, an enzyme of Thalictrum tuberosum, in particular the COMT described in SEQ ID NO: 88, an enzyme of Punica granatum, in particular the COMT described in SEQ ID NO: 89, an enzyme of Brassica cretica, in particular the COMT described in SEQ ID NO: 90, an enzyme of Lycium chinense, in particular the COMT described in SEQ ID NO: 91, or an enzyme of Acer yangbiense, in particular the COMT described in SEQ ID NO: 92.

Preferably, the COMT may be an enzyme of Panicum virgatum, in particular the COMT described in SEQ ID NO: 71 or 76, an enzyme of Triticum aestivum, in particular the COMT described in SEQ ID NO: 74, an enzyme of Zea mays, in particular the COMT described in SEQ ID NO: 79 or 82, or an enzyme of Stylosanthes humilis, in particular the COMT described in SEQ ID NO: 80.

According to one embodiment, the COMT comprises a sequence chosen from sequences SEQ ID NOs: 73 to 92, and polypeptides comprising a sequence having at least 60, 70, 80, 85, 90 or 95%, preferably at least 80, 85, 90 or 95%, sequence identity with one of these sequences and exhibiting COMT activity.

According to a particular embodiment, the COMT comprises a sequence chosen from sequences SEQ ID NOs: 73 to 81, and polypeptides comprising a sequence having at least 60, 70, 80, 85, 90 or 95%, preferably at least 80, 85, 90 or 95%, sequence identity with one of these sequences and exhibiting COMT activity.

According to a particular embodiment, the COMT comprises a sequence chosen from sequences SEQ ID NOs: 73 to 82, and polypeptides comprising a sequence having at least 60, 70, 80, 85, 90 or 95%, preferably at least 80, 85, 90 or 95%, sequence identity with one of these sequences and exhibiting COMT activity.

According to another particular embodiment, the COMT comprises a sequence chosen from sequences SEQ ID NOs: 72 to 74, 76 and 79 to 92 and polypeptides comprising a sequence having at least 95% sequence identity with one of these sequences and exhibiting COMT activity.

According to another particular embodiment, the COMT comprises a sequence chosen from sequences SEQ ID NOs: 72 to 74, 76, 79 to 89, 91 and 92 and polypeptides comprising a sequence having at least 90 or 95% sequence identity with one of these sequences and exhibiting COMT activity.

According to another particular embodiment, the COMT comprises a sequence chosen from sequences SEQ ID NOs: 72 to 74, 76, 79 to 83, 85 to 89, 91 and 92 and polypeptides comprising a sequence having at least 80, 85, 90 or 95% sequence identity with any of these sequences and exhibiting COMT activity.

According to a preferred embodiment, the COMT comprises a sequence chosen from sequences SEQ ID NOs: 74, 76, 79, 80 and 82 and polypeptides comprising a sequence having at least 80, 85, 90 or 95% sequence identity with any of these sequences and exhibiting COMT activity.

According to another preferred embodiment, the COMT comprises a sequence chosen from sequences SEQ ID NOs: 74, 76, 79 and 82 and polypeptides comprising a sequence having at least 80, 85, 90 or 95% sequence identity with any of these sequences and exhibiting COMT activity.

According to another preferred embodiment, the COMT comprises a sequence chosen from sequences SEQ ID NOs: 74, 76, 79 and 80 and polypeptides comprising a sequence having at least 80, 85, 90 or 95% sequence identity with any of these sequences and exhibiting COMT activity.

According to a particularly preferred embodiment, the COMT comprises a sequence chosen from sequences SEQ ID NOs: 76 and 80 and polypeptides comprising a sequence having at least 80, 85, 90 or 95% sequence identity with any of these sequences and exhibiting COMT activity.

The recombinant microorganism according to the invention may also comprise one or more enzymes necessary for the production of caffeic acid from p-coumaric acid. Thus, the microorganism may further comprise (i) a heterologous nucleic acid sequence coding for a 4-hydroxyphenylacetate 3-monooxygenase oxygenase (HpaB) and a heterologous nucleic acid sequence coding for a 4-hydroxyphenylacetate 3-monooxygenase reductase (HpaC) and/or (ii) a heterologous nucleic acid sequence coding for a CPR-dependent p-coumarate 3-hydroxylase (C3H).

These enzymes may be as defined above.

In one embodiment, the recombinant microorganism comprises a heterologous nucleic acid sequence coding for a 4-hydroxyphenylacetate 3-monooxygenase oxygenase (HpaB) and a heterologous nucleic acid sequence coding for a 4-hydroxyphenylacetate 3-monooxygenase reductase (HpaC).

In particular, the HpaB enzyme may comprise a sequence chosen from sequences SEQ ID NOs: 17 and 26 and polypeptides comprising a sequence having at least 60, 70, 80, 85, 90 or 95%, preferably at least 80, 85, 90 or 95%, sequence identity with any of these sequences and exhibiting 4-hydroxyphenylacetate 3-monooxygenase oxygenase activity. Preferably, the HpaB enzyme comprises a sequence chosen from SEQ ID No: 17 and polypeptides comprising a sequence having at least 60, 70, 80, 85, 90 or 95%, preferably at least 80, 85, 90 or 95%, sequence identity with SEQ ID NO: 17 and exhibiting 4-hydroxyphenylacetate 3-monooxygenase oxygenase activity.

The HpaC enzyme may comprise a sequence chosen from sequences SEQ ID NOs: 18 and 27 and polypeptides comprising a sequence having at least 60, 70, 80, 85, 90 or 95%, preferably at least 80, 85, 90 or 95%, sequence identity with any of these sequences and exhibiting 4-hydroxyphenylacetate 3-monooxygenase reductase activity. Preferably, the HpaC enzyme comprises a sequence chosen from SEQ ID NO: 18 and polypeptides comprising a sequence having at least 60, 70, 80, 85, 90 or 95%, preferably at least 80, 85, 90 or 95%, sequence identity with SEQ ID NO: 18 and exhibiting a 4-hydroxyphenylacetate 3-monooxygenase reductase activity.

According to a particular embodiment, the recombinant microorganism comprises
a heterologous nucleic acid sequence coding for a COMT as defined above, preferably a COMT comprising a sequence chosen from sequences SEQ ID NOs: 74, 76, 79, 80 and 82 and polypeptides comprising a sequence having at least 80, 85, 90 or 95% sequence identity with any of these sequences and exhibiting COMT activity; and
a heterologous nucleic acid sequence coding for a 4-hydroxyphenylacetate 3-monooxygenase oxygenase, preferably comprising a sequence chosen from the sequences SEQ ID NOs: 17 and 26 and polypeptides comprising a sequence having at least 60, 70, 80, 85, 90 or 95%, preferably at least 80, 85, 90 or 95%, sequence identity with any of these sequences and exhibiting 4-hydroxyphenylacetate 3-monooxygenase oxygenase activity, and more particularly preferably, a sequence chosen from SEQ ID NOs: 17 and polypeptides comprising a sequence having at least 60, 70, 80, 85, 90 or 95%, preferably at least 80, 85, 90 or 95%, sequence identity with SEQ ID NO: 17 and exhibiting a 4-hydroxyphenylacetate 3-monooxygenase oxygenase activity, and
a heterologous nucleic acid sequence coding for a 4-hydroxyphenylacetate 3-monooxygenase reductase, preferably comprising a sequence chosen from sequences SEQ ID NOs: 18 and 27 and polypeptides comprising a sequence having at least 60, 70, 80, 85, 90 or 95%, preferably at least 80, 85, 90 or 95%, sequence identity with one of these sequences and exhibiting 4-hydroxyphenylacetate 3-monooxygenase reductase activity, and more particularly preferably, a sequence chosen from SEQ ID NOs: 18 and polypeptides comprising a sequence having at least 60, 70, 80, 85, 90 or 95%, preferably at least 80, 85, 90 or 95%, sequence identity with SEQ ID NO: 18 and exhibiting a 4-hydroxyphenylacetate 3-monooxygenase reductase activity.

According to a preferred embodiment, the recombinant microorganism comprises
a heterologous nucleic acid sequence coding for a COMT as defined above, preferably a COMT comprising a sequence chosen from sequences SEQ ID NOs: 74, 76, 79, 80 and 82 and polypeptides comprising a sequence having at least 80, 85, 90 or 95% sequence identity with any of these sequences and exhibiting COMT activity; and
a heterologous nucleic acid sequence coding for a 4-hydroxyphenylacetate 3-monooxygenase oxygenase comprising a sequence chosen from SEQ ID NOs: 17 and polypeptides comprising a sequence having at least 60, 70, 80, 85, 90 or 95%, preferably at least 80, 85, 90 or 95%, sequence identity with SEQ ID NO: 17 and exhibiting a 4-hydroxyphenylacetate 3-monooxygenase oxygenase activity, and
a heterologous nucleic acid sequence coding for a 4-hydroxyphenylacetate 3-monooxygenase reductase comprising a sequence chosen from SEQ ID NOs: 18 and polypeptides comprising a sequence having at least 60, 70, 80, 85, 90 or 95%, preferably at least 80, 85, 90 or 95%, sequence identity with SEQ ID NO: 18 and exhibiting a 4-hydroxyphenylacetate 3-monooxygenase reductase activity.

Alternatively or additionally, the recombinant microorganism may comprise a heterologous nucleic acid sequence coding for a CPR-dependent p-coumarate 3-hydroxylase (C3H). The C3H enzyme may comprise a sequence chosen from SEQ ID NOs: 25 and polypeptides comprising a sequence having at least 60, 70, 80, 85, 90 or 95%, preferably at least 80, 85, 90 or 95%, sequence identity with SEQ ID NO: 25 and exhibiting a p-coumarate 3-hydroxylase activity.

The recombinant microorganism according to the invention may also comprise one or more enzymes necessary for the production of p-coumaric acid from tyrosine. Thus, the microorganism may also comprise a heterologous nucleic acid sequence coding for a tyrosine ammonia lyase (TAL). This enzyme may be as defined above.

In particular, the TAL may comprise a sequence chosen from SEQ ID NOs: 19, 30 and 68 and polypeptides comprising a sequence having at least 60, 70, 80, 85, 90 or 95%, preferably at least 80, 85, 90 or 95%, sequence identity with SEQ ID NO: 19, 30 or 68 and exhibiting tyrosine ammonia lyase activity. Preferably, the TAL comprises a sequence chosen from SEQ ID NO: 19 and polypeptides comprising a sequence having at least 60, 70, 80, 85, 90 or 95%, preferably at least 80, 85, 90 or 95%, sequence identity with SEQ ID NO: 19, and exhibiting tyrosine ammonia lyase activity.

According to a particular embodiment, the recombinant microorganism comprises
a heterologous nucleic acid sequence coding for a COMT as defined above, preferably a COMT comprising a sequence chosen from sequences SEQ ID NOs: 74, 76, 79, 80 and 82 and polypeptides comprising a sequence having at least 80, 85, 90 or 95% sequence identity with any of these sequences and exhibiting COMT activity; and
a heterologous nucleic acid sequence coding for a tyrosine ammonia lyase (TAL), preferably comprising a sequence chosen from SEQ ID NOs: 19, 30 and 68 and polypeptides comprising a sequence having at least 60, 70, 80, 85, 90 or 95%, preferably at least 80, 85, 90 or 95%, sequence identity with SEQ ID NO: 19, 30 or 68 and exhibiting tyrosine ammonia lyase activity, and more particularly preferably, a sequence chosen from SEQ ID NO: 19 and polypeptides comprising a sequence having at least 60, 70, 80, 85, 90 or 95%, preferably at least 80, 85, 90 or 95%, sequence identity with SEQ ID NO: 19, and exhibiting tyrosine ammonia lyase activity; and
(i)—a heterologous nucleic acid sequence coding for a 4-hydroxyphenylacetate 3-monooxygenase oxygenase, preferably comprising a sequence chosen from sequences SEQ ID NOs: 17 and 26 and polypeptides comprising a sequence having at least 60, 70, 80, 85, 90 or 95%, preferably at least 80, 85, 90 or 95%, sequence identity with one of these sequences and exhibiting 4-hydroxyphenylacetate 3-monooxygenase oxygenase activity, and more particularly preferably, a sequence chosen from SEQ ID NO: 17 and polypeptides comprising a sequence having at least 60, 70, 80, 85, 90 or 95%, preferably at least 80, 85, 90 or 95%, sequence identity with SEQ ID NO: 17 and exhibiting a 4-hydroxyphenylacetate 3-monooxygenase oxygenase activity, and
a heterologous nucleic acid sequence coding for a 4-hydroxyphenylacetate 3-monooxygenase reductase, preferably comprising a sequence chosen from sequences SEQ ID NOs: 18 and 27 and polypeptides comprising a sequence having at least 60, 70, 80, 85, 90 or 95%, preferably at least 80, 85, 90 or 95%, sequence identity with one of these sequences and exhibiting 4-hydroxyphenylacetate 3-monooxygenase reductase activity, and more particularly preferably, a sequence chosen from SEQ ID NO: 18 and polypeptides comprising a sequence having at least 60, 70, 80, 85, 90 or 95%, preferably at least 80, 85, 90 or 95%, sequence identity with SEQ ID NO: 18 and exhibiting a 4-hydroxyphenylacetate 3-monooxygenase reductase activity; and/or
(ii) a heterologous nucleic acid sequence coding for a CPR-dependent p-coumarate 3-hydroxylase, preferably comprising a sequence chosen from SEQ ID NO: 25 and polypeptides comprising a sequence having at least 60, 70, 80, 85, 90 or 95%, preferably at least 80, 85, 90 or 95%, sequence identity with SEQ ID NO: 25 and exhibiting a p-coumarate 3-hydroxylase activity.

According to a preferred embodiment, the recombinant microorganism comprises
a heterologous nucleic acid sequence coding for a COMT as defined above, preferably a COMT comprising a sequence chosen from sequences SEQ ID NOs: 74, 76, 79, 80 and 82 and polypeptides comprising a sequence having at least 80, 85, 90 or 95% sequence identity with any of these sequences and exhibiting COMT activity; and
a heterologous nucleic acid sequence coding for a tyrosine ammonia lyase (TAL) comprising a sequence chosen from SEQ ID NO: 19 and polypeptides comprising a sequence having at least 60, 70, 80, 85, 90 or 95%, preferably at least 80, 85, 90 or 95%, sequence identity with SEQ ID NO: 19, and exhibiting tyrosine ammonia lyase activity; and
a heterologous nucleic acid sequence coding for a 4-hydroxyphenylacetate 3-monooxygenase oxygenase comprising a sequence chosen from SEQ ID NOs: 17 and polypeptides comprising a sequence having at least 60, 70, 80, 85, 90 or 95%, preferably at least 80, 85, 90 or 95%, sequence identity with SEQ ID NO: 17 and exhibiting a 4-hydroxyphenylacetate 3-monooxygenase oxygenase activity, and
a heterologous nucleic acid sequence coding for a 4-hydroxyphenylacetate 3-monooxygenase reductase comprising a sequence chosen from SEQ ID NOs: 18 and polypeptides comprising a sequence having at least 60, 70, 80, 85, 90 or 95%, preferably at least 80, 85, 90 or 95%, sequence identity with SEQ ID NO: 18 and exhibiting a 4-hydroxyphenylacetate 3-monooxygenase reductase activity.

The recombinant microorganism according to the invention may also comprise one or more enzymes necessary for the production of p-coumaric acid from phenylalanine. Thus, the microorganism may further comprise a heterologous nucleic acid sequence coding for a phenylalanine ammonia lyase (PAL) and a heterologous nucleic acid sequence coding for a cinnamate 4-hydroxylase (C4H). These enzymes may be as defined above.

In particular, the PAL may comprise a sequence chosen from SEQ ID NOs: 20, 69, 31 and 32 and polypeptides comprising a sequence having at least 60, 70, 80, 85, 90 or 95%, preferably at least 80, 85, 90 or 95%, sequence identity with SEQ ID NO: 20, 69, 31 or 32 and exhibiting a phenylalanine ammonia lyase activity. Preferably, the PAL comprises a sequence chosen from SEQ ID NO: 20 and polypeptides comprising a sequence having at least 60, 70, 80, 85, 90 or 95%, preferably at least 80, 85, 90 or 95%, sequence identity with SEQ ID NO: 20 and exhibiting a phenylalanine ammonia lyase activity.

The C4H may comprise a sequence chosen from SEQ ID NOs: 21, 33, 34 and 70 and polypeptides comprising a sequence having at least 60, 70, 80, 85, 90 or 95%, preferably at least 80, 85, 90 or 95%, sequence identity with SEQ ID NO: 21, 33, 34 or 70 and exhibiting cinnamate 4-hydroxylase activity. Preferably, the C4H comprises a sequence chosen from SEQ ID NO: 21 and polypeptides comprising a sequence having at least 60, 70, 80, 85, 90 or 95%, preferably at least 80, 85, 90 or 95%, sequence identity with SEQ ID NO: 21 and exhibiting cinnamate 4-hydroxylase activity.

According to a particular embodiment, the recombinant microorganism comprises
a heterologous nucleic acid sequence coding for a COMT as defined above, preferably a COMT comprising a sequence chosen from sequences SEQ ID NOs: 74, 76, 79, 80 and 82 and polypeptides comprising a sequence having at least 80, 85, 90 or 95% sequence identity with any of these sequences and exhibiting COMT activity; and
a heterologous nucleic acid sequence coding for phenylalanine ammonia lyase, preferably comprising a sequence chosen from SEQ ID NOs: 20, 69, 31 and 32 and polypeptides comprising a sequence having at least 60, 70, 80, 85, 90 or 95%, preferably at least 80, 85, 90 or 95%, sequence identity with SEQ ID NO: 20, 69, 31 or 32 and exhibiting phenylalanine ammonia lyase activity, and more particularly preferably, a sequence chosen from SEQ ID NO: 20 and polypeptides comprising a sequence having at least 60, 70, 80, 85, 90 or 95%, preferably at least 80, 85, 90 or 95%, sequence identity with SEQ ID NO: 20 and exhibiting phenylalanine ammonia lyase activity; and
a heterologous nucleic acid sequence coding for a cinnamate 4-hydroxylase, preferably comprising a sequence chosen from SEQ ID NOs: 21, 33, 34 and 70 and polypeptides comprising a sequence having at least 60, 70, 80, 85, 90 or 95%, preferably at least 80, 85, 90 or 95%, sequence identity with sequence SEQ ID NOs: 21, 33, 34 or 70 and exhibiting cinnamate 4-hydroxylase activity, and more particularly preferably, a sequence chosen from SEQ ID NO: 21 and polypeptides comprising a sequence having at least 60, 70, 80, 85, 90 or 95%, preferably at least 80, 85, 90 or 95%, sequence identity with SEQ ID NO: 21 and exhibiting cinnamate 4-hydroxylase activity; and
(i)—a heterologous nucleic acid sequence coding for a 4-hydroxyphenylacetate 3-monooxygenase oxygenase, preferably comprising a sequence chosen from sequences SEQ ID NOs: 17 and 26 and polypeptides comprising a sequence having at least 60, 70, 80, 85, 90 or 95%, preferably at least 80, 85, 90 or 95%, sequence identity with one of these sequences and exhibiting 4-hydroxyphenylacetate 3-monooxygenase oxygenase activity, and more particularly preferably, a sequence chosen from SEQ ID NO: 17 and polypeptides comprising a sequence having at least 60, 70, 80, 85, 90 or 95%, preferably at least 80, 85, 90 or 95%, sequence identity with SEQ ID NO: 17 and exhibiting a 4-hydroxyphenylacetate 3-monooxygenase oxygenase activity, and
a heterologous nucleic acid sequence coding for a 4-hydroxyphenylacetate 3-monooxygenase reductase, preferably comprising a sequence chosen from sequences SEQ ID NOs: 18 and 27 and polypeptides comprising a sequence having at least 60, 70, 80, 85, 90 or 95%, preferably at least 80, 85, 90 or 95%, sequence identity with one of these sequences and exhibiting 4-hydroxyphenylacetate 3-monooxygenase reductase activity, and more particularly preferably, a sequence chosen from SEQ ID NO: 18 and polypeptides comprising a sequence having at least 60, 70, 80, 85, 90 or 95%, preferably at least 80, 85, 90 or 95%, sequence identity with SEQ ID NO: 18 and exhibiting a 4-hydroxyphenylacetate 3-monooxygenase reductase activity; and/or
(ii) a heterologous nucleic acid sequence coding for a CPR-dependent p-coumarate 3-hydroxylase, preferably comprising a sequence chosen from SEQ ID NO: 25 and polypeptides comprising a sequence having at least 60, 70, 80, 85, 90 or 95%, preferably at least 80, 85, 90 or 95%, sequence identity with SEQ ID NO: 25 and exhibiting a p-coumarate 3-hydroxylase activity.

Preferably, in this embodiment, the microorganism also comprises a heterologous nucleic acid sequence coding for a tyrosine ammonia lyase (TAL), preferably comprising a sequence chosen from SEQ ID NOs: 19, 30 and 68 and polypeptides comprising a sequence having at least 60, 70, 80, 85, 90 or 95%, preferably at least 80, 85, 90 or 95%, sequence identity with sequence SEQ ID NOs: 19, 30 or 68 and exhibiting tyrosine ammonia lyase activity, and more particularly preferably, a sequence chosen from SEQ ID NO: 19 and polypeptides comprising a sequence having at least 60, 70, 80, 85, 90 or 95%, preferably at least 80, 85, 90 or 95%, sequence identity with sequence SEQ ID NO: 19, and exhibiting tyrosine ammonia lyase activity.

According to a preferred embodiment, the recombinant microorganism comprises
a heterologous nucleic acid sequence coding for a COMT as defined above, preferably a COMT comprising a sequence chosen from sequences SEQ ID NOs: 74, 76, 79, 80 and 82 and polypeptides comprising a sequence having at least 80, 85, 90 or 95% sequence identity with any of these sequences and exhibiting COMT activity; and
a heterologous nucleic acid sequence coding for a phenylalanine ammonia lyase comprising a sequence chosen from SEQ ID NO: 20 and polypeptides comprising a sequence having at least 60, 70, 80, 85, 90 or 95%, preferably at least 80, 85, 90 or 95%, sequence identity with SEQ ID NO: 20 and exhibiting a phenylalanine ammonia lyase activity; and
a heterologous nucleic acid sequence coding for a cinnamate 4-hydroxylase comprising a sequence chosen from SEQ ID NO: 21 and polypeptides comprising a sequence having at least 60, 70, 80, 85, 90 or 95%, preferably at least 80, 85, 90 or 95%, sequence identity with sequence SEQ ID NO: 21 and exhibiting cinnamate 4-hydroxylase activity; and
a heterologous nucleic acid sequence coding for a 4-hydroxyphenylacetate 3-monooxygenase oxygenase comprising a sequence chosen from SEQ ID NOs: 17 and polypeptides comprising a sequence having at least 60, 70, 80, 85, 90 or 95%, preferably at least 80, 85, 90 or 95%, sequence identity with SEQ ID NO: 17 and exhibiting a 4-hydroxyphenylacetate 3-monooxygenase oxygenase activity, and
a heterologous nucleic acid sequence coding for a 4-hydroxyphenylacetate 3-monooxygenase reductase comprising a sequence chosen from SEQ ID NOs: 18 and polypeptides comprising a sequence having at least 60, 70, 80, 85, 90 or 95%, preferably at least 80, 85, 90 or 95%, sequence identity with sequence SEQ ID NO: 18 and exhibiting a 4-hydroxyphenylacetate 3-monooxygenase reductase activity.

Preferably, in this embodiment, the microorganism also comprises a heterologous nucleic acid sequence coding for a tyrosine ammonia lyase (TAL) and comprising a sequence chosen from SEQ ID NO: 19 and polypeptides comprising a sequence having at least 60, 70, 80, 85, 90 or 95%, preferably at least 80, 85, 90 or 95%, sequence identity with sequence SEQ ID NO: 19, and exhibiting a tyrosine ammonia lyase activity.

The recombinant microorganism according to the invention comprising a heterologous nucleic acid sequence coding for a COMT and optionally a heterologous nucleic acid sequence coding for a 4-hydroxyphenylacetate 3-monooxygenase oxygenase, a heterologous nucleic acid sequence coding for a 4-hydroxyphenylacetate 3-monooxygenase reductase, a heterologous nucleic acid sequence coding for C3H, a heterologous nucleic acid sequence coding for TAL, a heterologous nucleic acid sequence coding for PAL, and/or a heterologous nucleic acid sequence coding for C4H, may further comprise an endogenous nucleic acid coding for a cytochrome P450 reductase. Optionally, the endogenous CPR may be overexpressed, for example by replacing the promoter of the endogenous gene with a strong heterologous promoter and/or by increasing the number of copies of the endogenous gene. Alternatively, or in addition to this endogenous nucleic acid, the recombinant microorganism may comprise a heterologous nucleic acid sequence that codes for a cytochrome P450 reductase (CPR). This enzyme may be as defined above. In particular, the CPR may comprise a sequence chosen from SEQ ID NOs: 22, 35, 36, 37 and 38 and polypeptides comprising a sequence having at least 60, 70, 80, 85, 90 or 95%, preferably at least 80, 85, 90 or 95%, sequence identity with one of these sequences and exhibiting cytochrome P450 reductase activity. Preferably, the CPR comprises a sequence chosen from SEQ ID NO: 22 and polypeptides comprising a sequence having at least 60, 70, 80, 85, 90 or 95%, preferably at least 80, 85, 90 or 95%, sequence identity with sequence SEQ ID NO: 22 and exhibiting a cytochrome P450 reductase activity.

The recombinant microorganism according to the invention may also comprise the genetic modifications described above in order to increase the production of tyrosine and/or phenylalanine. In particular, it may comprise a heterologous nucleic acid sequence coding for a tyrosine feedback-resistant 3-deoxy-D-arabino-hepturosonate-7-phosphate (DAHP) synthase and/or a heterologous nucleic acid sequence coding for a tyrosine feedback-resistant chorismate mutase. Alternatively or additionally, an endogenous gene coding for a phenylpyruvate decarboxylase can be inactivated in said microorganism. The recombinant microorganism according to the invention can also be modified to inactivate the endogenous gene or genes coding for a ferulic acid decarboxylase as described above.

The recombinant microorganism according to the invention can be a eukaryotic or prokaryotic microorganism as defined above, preferably a yeast or a bacterium. In particular, the microorganism may be chosen from an Escherichia coli bacterium and a Saccharomyces cerevisiae yeast.

### Recombinant Nucleic Acid, Cassette and Expression Vector

Each nucleic acid sequence coding for an enzyme as described previously is included in an expression cassette. Preferably, the coding nucleic acid sequences have been optimized for expression in the host microorganism. The coding nucleic acid sequence is operatively linked to the elements required for the expression of the gene, notably for transcription and translation. These elements are chosen so as to be functional in the host recombinant microorganism. These elements may include, for example, transcription promoters, transcription activators, terminator sequences, and start and stop codons. The methods for selecting these elements as a function of the host cell in which expression is desired are well known to those skilled in the art.

Preferably, the promoter is a strong promoter. The promoter may be constitutive or inducible, preferably constitutive. A promoter can control the expression of one or more nucleic acid sequences coding for one or more enzymes as described above. For example, if the microorganism is prokaryotic, the promoter may be chosen from the following promoters: Lacl, LacZ, pLacT, ptac, pARA, pBAD, the RNA polymerase promoters of bacteriophage T3 or T7, the polyhedrin promoter, the PR or PL promoter of lambda phage. In one particular embodiment, the promoter is pLac. If the microorganism is eukaryotic and in particular a yeast, the promoter may be chosen from the following promoters: the promoter pTDH3, the promoter pTEF1, the promoter pTEF2, the promoter pCCW12, the promoter pHHF2, the promoter pHTB2 and the promoter pRPL18B. Examples of inducible promoters which can be used in yeast are the promoters tetO-2, GAL10, GAL10-CYC1 and PHO5.

All or part of the expression cassettes comprising the nucleic acid sequences coding for the enzymes as described or a combination of some of them may be included in a common expression vector or in different expression vectors.

The present invention therefore also relates to a vector comprising
a nucleic acid sequence coding for a 4-coumaroyl-CoA ligase (4CL), a nucleic acid sequence coding for a coumaroyl-CoA 3-hydroxylase (CCoA3H) and a nucleic acid sequence coding for an acyl-coenzyme A thioesterase and/or
at least one nucleic acid sequence chosen from a nucleic acid sequence coding for a caffeoyl-CoA O-methyltransferase (CCoAMT), a nucleic acid sequence coding for a caffeic acid O-methyltransferase (COMT), a nucleic acid sequence coding for a CCR, a nucleic acid sequence coding for an ALDH, a nucleic acid sequence coding for a p-coumarate 3-hydroxylase, a nucleic acid sequence coding for a 4-hydroxyphenylacetate 3-monooxygenase oxygenase, a nucleic acid sequence coding for a 4-hydroxyphenylacetate 3-monooxygenase reductase, a nucleic acid sequence coding for a tyrosine ammonia lyase (TAL), a nucleic acid sequence coding for a phenylalanine ammonia lyase (PAL), a nucleic acid sequence coding for a cinnamate 4-hydroxylase (C4H) and a nucleic acid sequence coding for a cytochrome P450 reductase (CPR), each of these enzymes being as defined above, and combinations thereof.

Preferably, the vector comprises
a nucleic acid sequence coding for a 4-coumaroyl-CoA ligase (4CL), a nucleic acid sequence coding for a coumaroyl-CoA 3-hydroxylase (CCoA3H) and a nucleic acid sequence coding for an acyl-coenzyme A thioesterase, and
at least one nucleic acid sequence chosen from a nucleic acid sequence coding for a caffeoyl-CoA O-methyltransferase (CCoAMT), a nucleic acid sequence coding for a caffeic acid O-methyltransferase (COMT), a nucleic acid sequence coding for a CCR, a nucleic acid sequence coding for an ALDH, a nucleic acid sequence coding for a p-coumarate 3-hydroxylase, a nucleic acid sequence coding for a 4-hydroxyphenylacetate 3-monooxygenase oxygenase, a nucleic acid sequence coding for a 4-hydroxyphenylacetate 3-monooxygenase reductase, a nucleic acid sequence coding for a tyrosine ammonia lyase (TAL), a nucleic acid sequence coding for a phenylalanine ammonia lyase (PAL), a nucleic acid sequence coding for a cinnamate 4-hydroxylase (C4H) and a nucleic acid sequence coding for a cytochrome P450 reductase (CPR), each of these enzymes being as defined above, and combinations thereof.

The present invention also relates to a vector comprising
a nucleic acid sequence coding for caffeic acid O-methyltransferase (COMT), and
at least one nucleic acid sequence chosen from a nucleic acid sequence coding for a p-coumarate 3-hydroxylase, a nucleic acid sequence coding for a 4-hydroxyphenylacetate 3-monooxygenase oxygenase, a nucleic acid sequence coding for a 4-hydroxyphenylacetate 3-monooxygenase reductase, a nucleic acid sequence coding for a tyrosine ammonia lyase (TAL), a nucleic acid sequence coding for a phenylalanine ammonia lyase (PAL), a nucleic acid sequence coding for a cinnamate 4-hydroxylase (C4H) and a nucleic acid sequence coding for a cytochrome P450 reductase (CPR), each of these enzymes being as defined above, and combinations thereof.

The vector may notably comprise combinations of particular coding sequences as described above.

The term “comprising a nucleic acid sequence” is preferably understood to mean “comprising an expression cassette comprising the nucleic acid sequence”. The vectors comprise heterologous coding sequences insofar as the coding sequences can be optimized for the host microorganism, be under the control of heterologous promoter(s) and/or may combine coding sequences that do not originate from the same organism of origin and/or that are not present in the same arrangement.

The vector may be any DNA sequence in which it is possible to insert foreign nucleic acids, the vectors making it possible to introduce foreign DNA into the host microorganism. For example, the vector may be a plasmid, a phagemid, a cosmid, an artificial chromosome, notably a YAC, or a BAC.

The expression vectors may further comprise nucleic acid sequences coding for selection markers. The selection markers may be genes for resistance to one or more antibiotics or auxotrophic genes. The auxotrophic gene may be, for example, HIS5, URA3, LEU2 or TRP1. The antibiotic resistance gene may, for example, preferably be a gene for resistance to ampicillin, chloramphenicol, spectinomycin, streptomycin, kanamycin, hygromycin, geneticin, fluoroacetamide, fluorocitrate, phleomycin, amphotericin-B and/or nourseothricin.

The introduction of vectors into a host microorganism is a process that is widely known to those skilled in the art. Several methods are described in particular in “Current Protocols in Molecular Biology”, 13.7.1-13.7.10; or else in Ellis T. et aL., Integrative Biology, 2011, 3(2), 109-118.

The host microorganism can be transiently or stably transformed/transfected and the nucleic acid, the cassette or the vector according to the invention can be contained therein in the form of an episome or in a form integrated into the genome of the host cell. The expression vector may also comprise one or more sequences allowing the targeted insertion of the vector, the expression cassette or the nucleic acid into the genome of the host cell.

All or part of the expression cassettes comprising the nucleic acid sequences coding for the enzymes as described above may be inserted into the/a chromosome of the recombinant microorganism. On the contrary, all or part of the expression cassettes comprising the nucleic acid sequences coding for the enzymes as described may be conserved in episomal form, in particular in plasmid form.

Optionally, the microorganism may comprise a plurality of copies of nucleic acid sequences coding for an enzyme as described above. Notably, it may comprise 2 to 10 copies, for example 2, 3, 4, 5, 6, 7, 8, 9 or 10 copies of a nucleic acid sequence coding for an enzyme as described previously.

Optionally, the host microorganism can be transformed/transfected with several vectors according to the invention, identical or different. It can also be transformed/transfected with one or more other vectors coding for example for other enzymes necessary for the production of carboxylic acid.

The present invention also relates to a method for preparing a recombinant microorganism according to the present invention, comprising introducing a vector as defined above into the microorganism and selecting the microorganisms comprising said vector.

It also relates to a method for preparing a microorganism according to the present invention, comprising introducing
a nucleic acid sequence coding for a 4-coumaroyl-CoA ligase (4CL), a nucleic acid sequence coding for a coumaroyl-CoA 3-hydroxylase (CCoA3H) and a nucleic acid sequence coding for an acyl-coenzyme A thioesterase,
and optionally at least one nucleic acid sequence chosen from a nucleic acid sequence coding for a caffeoyl-CoA O-methyltransferase (CCoAMT), a nucleic acid sequence coding for a caffeic acid O-methyltransferase (COMT), a nucleic acid sequence coding for a CCR, a nucleic acid sequence coding for an ALDH, a nucleic acid sequence coding for a p-coumarate 3-hydroxylase, a nucleic acid sequence coding for a 4-hydroxyphenylacetate 3-monooxygenase oxygenase, a nucleic acid sequence coding for a 4-hydroxyphenylacetate 3-monooxygenase reductase, a nucleic acid sequence coding for a tyrosine ammonia lyase (TAL), a nucleic acid sequence coding for a phenylalanine ammonia lyase (PAL), a nucleic acid sequence coding for a cinnamate 4-hydroxylase (C4H) and a nucleic acid sequence coding for a cytochrome P450 reductase (CPR), each of these enzymes being as defined above, and combinations thereof, and selecting microorganisms comprising said nucleic acid sequences.

Preferably, the method of preparing a microorganism according to the present invention comprises introducing
a nucleic acid sequence coding for a 4-coumaroyl-CoA ligase (4CL), a nucleic acid sequence coding for a coumaroyl-CoA 3-hydroxylase (CCoA3H) and a nucleic acid sequence coding for an acyl-coenzyme A thioesterase
and optionally at least one nucleic acid sequence chosen from a nucleic acid sequence coding for a caffeoyl-CoA O-methyltransferase (CCoAMT) and a nucleic acid sequence coding for a caffeic acid O-methyltransferase (COMT), or a combination of both, and selecting microorganisms comprising said nucleic acid sequences, each of these enzymes being as defined above.

The present invention also relates to a method for preparing a microorganism according to the present invention, comprising a nucleic acid sequence coding for caffeic acid O-methyltransferase, the method comprising introducing
a nucleic acid sequence coding for caffeic acid O-methyltransferase (COMT), and
optionally, at least one nucleic acid sequence chosen from a nucleic acid sequence coding for a p-coumarate 3-hydroxylase, a nucleic acid sequence coding for a 4-hydroxyphenylacetate 3-monooxygenase oxygenase, a nucleic acid sequence coding for a 4-hydroxyphenylacetate 3-monooxygenase reductase, a nucleic acid sequence coding for a tyrosine ammonia lyase (TAL), a nucleic acid sequence coding for a phenylalanine ammonia lyase (PAL), a nucleic acid sequence coding for a cinnamate 4-hydroxylase (C4H) and a nucleic acid sequence coding for a cytochrome P450 reductase (CPR), each of these enzymes being as defined above, and combinations thereof, and selecting microorganisms comprising said nucleic acid sequences.

Preferably, the method of preparing a microorganism according to the present invention comprising a nucleic acid sequence coding for caffeic acid O-methyltransferase comprises introducing a nucleic acid sequence coding for caffeic acid O-methyltransferase (COMT), a nucleic acid sequence coding for a 4-hydroxyphenylacetate 3-monooxygenase oxygenase, a nucleic acid sequence coding for a 4-hydroxyphenylacetate 3-monooxygenase reductase, a nucleic acid sequence coding for a tyrosine ammonia lyase (TAL), a nucleic acid sequence coding for a phenylalanine ammonia lyase (PAL), a nucleic acid sequence coding for a cinnamate 4-hydroxylase (C4H) and a nucleic acid sequence coding for a cytochrome P450 reductase (CPR), each of these enzymes being as defined above, and combinations thereof, and selecting microorganisms comprising said nucleic acid sequences.

### Uses of the Recombinant Microorganism

The present invention also relates to the use of a microorganism according to the invention, namely a recombinant microorganism as described above and genetically modified to produce phenylpropanoids from p-coumaric acid and via intermediate compounds which are carboxyl-CoAs, to produce a phenylpropanoid chosen from caffeic acid and ferulic acid. It also relates to a method for producing a phenylpropanoid chosen from caffeic acid and ferulic acid, comprising culturing said microorganism according to the invention and optionally harvesting and/or purifying said phenylpropanoid.

Preferably, the phenylpropanoid is ferulic acid.

The present invention also relates to the use of a microorganism according to the invention, namely a recombinant microorganism comprising a heterologous nucleic acid sequence coding for a COMT as described above, for producing ferulic acid. It also relates to a method for producing ferulic acid, comprising culturing said microorganism according to the invention and optionally harvesting and/or purifying said ferulic acid.

The compound produced by the method according to the invention can be either the final product or a synthesis or biosynthesis intermediate for the preparation of other compounds.

The conditions for cultivating the microorganism according to the invention may be adapted according to the conventional techniques that are well known to those skilled in the art.

The microorganism is cultivated in a suitable culture medium. The term “suitable culture medium” generally denotes a culture medium providing the nutrients that are essential for or beneficial to the maintenance and/or growth of said microorganism, such as carbon sources; nitrogen sources such as ammonium sulfate; phosphorus sources, for example monobasic potassium phosphate; trace elements, for example copper, iodide, iron, magnesium, zinc or molybdate salts; vitamins and other growth factors such as amino acids or other growth promoters. An antifoam may be added if need be. According to the invention, this suitable culture medium may be chemically defined or “undefined”. The culture medium may thus have a composition identical to or similar to a synthetic medium, as defined by Verduyn et aL., (Yeast. 1992. 8:501-17), adapted by Visser et aL., (Biotechnology and bioengineering. 2002. 79:674-81), or commercially available such as the YNB medium (Yeast Nitrogen Base, MP Biomedicals or Sigma-Aldrich). Notably, the culture medium may comprise a simple carbon source, such as glucose, fructose, xylose, ethanol, glycerol, galactose, sucrose, cellulose, cellobiose, starch, glucose polymers, molasses, or byproducts of these sugars. The “undefined” medium may be a liquid medium composed of hydrolysates of microorganisms and/or proteins, for example and not exclusively yeast extract and/or peptones. Adding to this composition usually is a simple carbon source, such as glucose, fructose, xylose, ethanol, glycerol, galactose, sucrose, cellulose, cellobiose, starch, glucose polymers, molasses, or byproducts of these sugars. The microorganism according to the invention may comprise all or part of the phenylpropanoid biosynthetic pathway. Thus, production can be carried out in the presence of a simple carbon source such as glucose, or in the presence of a synthetic intermediate such as tyrosine, phenylalanine, cinnamic acid or p-coumaric acid.

According to one embodiment, the microorganism according to the invention is used in a method for producing caffeic acid. Preferably, in this embodiment, the microorganism comprises (i) a heterologous nucleic acid sequence coding for a 4-coumaroyl-CoA ligase (4CL), a heterologous nucleic acid sequence coding for a coumaroyl-CoA 3-hydroxylase (CCoA3H), a heterologous nucleic acid sequence coding for an acyl-coenzyme A thioesterase.

The microorganism may further comprise a heterologous nucleic acid sequence coding for a C3H, a heterologous nucleic acid sequence coding for a 4-methoxybenzoate O-demethylase, a heterologous nucleic acid sequence coding for a TAL, a heterologous nucleic acid sequence coding for a phenylalanine ammonia lyase (PAL), a heterologous nucleic acid sequence coding for a cinnamate 4-hydroxylase (C4H), a heterologous nucleic acid sequence coding for a 4-hydroxyphenylacetate 3-monooxygenase oxygenase, and/or a heterologous nucleic acid sequence coding for a 4-hydroxyphenylacetate 3-monooxygenase reductase.

Preferably, the microorganism further comprises a heterologous nucleic acid sequence coding for a TAL, a heterologous nucleic acid sequence coding for a phenylalanine ammonia lyase (PAL), a heterologous nucleic acid sequence coding for a cinnamate 4-hydroxylase (C4H), a heterologous nucleic acid sequence coding for a 4-hydroxyphenylacetate 3-monooxygenase oxygenase, and a heterologous nucleic acid sequence coding for a 4-hydroxyphenylacetate 3-monooxygenase reductase. The enzymes and combinations of enzymes are as described above.

Preferably, the cultivation of the microorganism is carried out without the addition of intermediate compound to the medium, i.e. without the addition of tyrosine, phenylalanine, cinnamic acid, p-coumaric acid, L-DOPA and/or caffeoyl-CoA. Preferably, in this embodiment, the microorganism comprises
a heterologous nucleic acid sequence coding for a 4-coumaroyl-CoA ligase (4CL), a heterologous nucleic acid sequence coding for a coumaroyl-CoA 3-hydroxylase (CCoA3H) and a heterologous nucleic acid sequence coding for an acyl-coenzyme A thioesterase, and
a heterologous nucleic acid sequence coding for a tyrosine ammonia lyase (TAL), a heterologous nucleic acid sequence coding for a phenylalanine ammonia lyase (PAL) and a heterologous nucleic acid sequence coding for a cinnamate 4-hydroxylase (C4H), each of these enzymes being as defined above, and combinations thereof.

According to a particular embodiment, the microorganism according to the invention used in a method for producing caffeic acid comprises:
a heterologous nucleic acid sequence coding for a 4CL comprising a sequence chosen from sequences SEQ ID NOs: 5 to 9 and polypeptides comprising a sequence having at least 60, 70, 80, 85, 90 or 95% sequence identity with any of these sequences and exhibiting 4CL activity; preferably a 4CL comprising a sequence chosen from sequences SEQ ID NOs: 5, 6, 8 and 9 and polypeptides comprising a sequence having at least 60, 70, 80, 85, 90 or 95% sequence identity with any of these sequences and exhibiting 4CL activity, and more particularly preferably a 4CL comprising a sequence chosen from sequences SEQ ID NOs: 6 and 8 and polypeptides comprising a sequence having at least 60, 70, 80, 85, 90 or 95% sequence identity with any of these sequences and exhibiting 4CL activity; and
a heterologous nucleic acid sequence coding for a CCoA3H comprising a sequence chosen from sequences SEQ ID NOs: 42 to 67 and polypeptides comprising a sequence having at least 60, 70, 80, 85, 90 or 95% sequence identity with any of these sequences and exhibiting CCoA3H activity; preferably a CCoA3H comprising a sequence chosen from sequences SEQ ID NO: 42, 45 and 49 and polypeptides comprising a sequence having at least 60, 70, 80, 85, 90 or 95% sequence identity with any of these sequences and exhibiting CCoA3H activity, and more particularly preferably a CCoA3H comprising a sequence chosen from sequence SEQ ID NO: 49 and polypeptides comprising a sequence having at least 60, 70, 80, 85, 90 or 95% sequence identity with said sequence and exhibiting CCoA3H activity; and
a heterologous nucleic acid sequence coding for an acyl-coenzyme A thioesterase comprising a sequence chosen from sequences SEQ ID NOs: 1, 2 and 39, and polypeptides comprising a sequence having at least 60, 70, 80, 85, 90 or 95% sequence identity with any of these sequences and exhibiting acyl-coenzyme A thioesterase activity, preferably an acyl-coenzyme A thioesterase comprising a sequence chosen from sequences SEQ ID NOs: 2 and 39 and polypeptides comprising a sequence having at least 60, 70, 80, 85, 90 or 95% sequence identity with any of these sequences and exhibiting acyl-coenzyme A thioesterase activity; and
a heterologous nucleic acid sequence coding for a TAL comprising a sequence chosen from sequences SEQ ID NOs: 19, 30 and 68 and polypeptides comprising a sequence having at least 60, 70, 80, 85, 90 or 95% sequence identity with one of these sequences and exhibiting TAL activity, preferably a TAL comprising a sequence chosen from sequences SEQ ID NO: 19 and 68, preferably SEQ ID NO: 19, and polypeptides comprising a sequence having at least 60, 70, 80, 85, 90 or 95% sequence identity with any of these sequences, preferably with SEQ ID NO: 19, and exhibiting TAL activity, and
a heterologous nucleic acid sequence coding for a phenylalanine ammonia lyase (PAL) comprising a sequence chosen from SEQ ID NOs: 20, 69, 31 and 32 and polypeptides comprising a sequence having at least 60, 70, 80, 85, 90 or 95% sequence identity with any of these sequences and exhibiting PAL activity, and more particularly preferably, comprising a sequence chosen from sequences SEQ ID NOs: 20, 32 and 69, preferably SEQ ID NO: 20, and polypeptides comprising a sequence having at least 60, 70, 80, 85, 90 or 95% sequence identity with one of these sequences, preferably with SEQ ID NO: 20, and exhibiting PAL activity; and
a heterologous nucleic acid sequence coding for a cinnamate 4-hydroxylase (C4H) comprising a sequence chosen from SEQ ID NOs: 21, 70, 33 and 34 and polypeptides comprising a sequence having at least 60, 70, 80, 85, 90 or 95% sequence identity with any of these sequences and exhibiting C4H activity, and more particularly preferably, comprising a sequence chosen from sequences SEQ ID NOs: 21 and 70, preferably SEQ ID NO: 21, and polypeptides comprising a sequence having at least 60, 70, 80, 85, 90 or 95% sequence identity with any of these sequences, preferably with SEQ ID NO: 21, and exhibiting C4H activity.

According to a preferred embodiment, the microorganism according to the invention used in a method for producing caffeic acid comprises:
a heterologous nucleic acid sequence coding for a 4CL comprising a sequence chosen from sequences SEQ ID NOs: 6 and 8 and polypeptides comprising a sequence having at least 60, 70, 80, 85, 90 or 95% sequence identity with any of these sequences and exhibiting 4CL activity; and
a heterologous nucleic acid sequence coding for a CCoA3H comprising a sequence chosen from sequences SEQ ID NOs: 42, 45 and 49 and polypeptides comprising a sequence having at least 60, 70, 80, 85, 90 or 95% sequence identity with any of these sequences and exhibiting CCoA3H activity, and preferably a CCoA3H comprising a sequence chosen from sequence SEQ ID NO: 49 and polypeptides comprising a sequence having at least 60, 70, 80, 85, 90 or 95% sequence identity with said sequence and exhibiting CCoA3H activity; and
a heterologous nucleic acid sequence coding for an acyl-coenzyme A thioesterase comprising a sequence chosen from sequences SEQ ID NOs: 2 and 39 and polypeptides comprising a sequence having at least 60, 70, 80, 85, 90 or 95% sequence identity with any of these sequences and exhibiting acyl-coenzyme A thioesterase activity; and
a heterologous nucleic acid sequence coding for a TAL comprising a sequence chosen from sequences SEQ ID NOs: 19 and 68, preferably SEQ ID NO: 19, and polypeptides comprising a sequence having at least 60, 70, 80, 85, 90 or 95% sequence identity with any of these sequences, preferably with SEQ ID NO: 19, and exhibiting TAL activity; and
a heterologous nucleic acid sequence coding for a phenylalanine ammonia lyase (PAL) comprising a sequence chosen from sequences SEQ ID NOs: 20, 32 and 69, preferably SEQ ID NO: 20, and polypeptides comprising a sequence having at least 60, 70, 80, 85, 90 or 95% sequence identity with any of these sequences, preferably with SEQ ID NO: 20, and exhibiting PAL activity; and
a heterologous nucleic acid sequence coding for a cinnamate 4-hydroxylase (C4H) comprising a sequence chosen from sequences SEQ ID NOs: 21 and 70, preferably SEQ ID NO: 21, and polypeptides comprising a sequence having at least 60, 70, 80, 85, 90 or 95% sequence identity with one of these sequences, preferably with SEQ ID NO: 21, and exhibiting C4H activity.

Optionally, said microorganism may comprise
(i)—a heterologous nucleic acid sequence coding for a 4-hydroxyphenylacetate 3-monooxygenase oxygenase, preferably comprising a sequence chosen from sequences SEQ ID NOs: 17 and 26 and polypeptides comprising a sequence having at least 60, 70, 80, 85, 90 or 95% sequence identity with any of these sequences and exhibiting 4-hydroxyphenylacetate 3-monooxygenase oxygenase activity, and more particularly preferably a sequence chosen from SEQ ID NOs: 17 and polypeptides comprising a sequence having at least 60, 70, 80, 85, 90 or 95% sequence identity with sequence SEQ ID NO: 17 and exhibiting a 4-hydroxyphenylacetate 3-monooxygenase oxygenase activity, and
a heterologous nucleic acid sequence coding for a 4-hydroxyphenylacetate 3-monooxygenase reductase, preferably comprising a sequence chosen from sequences SEQ ID NOs: 18 and 27 and polypeptides comprising a sequence having at least 60, 70, 80, 85, 90 or 95% sequence identity with any of these sequences and exhibiting 4-hydroxyphenylacetate 3-monooxygenase reductase activity, and more particularly preferably a sequence chosen from SEQ ID NOs: 18 and polypeptides comprising a sequence having at least 60, 70, 80, 85, 90 or 95% sequence identity with sequence SEQ ID NO: 18 and exhibiting a 4-hydroxyphenylacetate 3-monooxygenase reductase activity; and/or
(ii) a heterologous nucleic acid sequence coding for a C3H, preferably comprising a sequence chosen from sequence SEQ ID NO: 25 and polypeptides comprising a sequence having at least 60, 70, 80, 85, 90 or 95% sequence identity with sequence SEQ ID NO: 25 and exhibiting p-coumarate 3-hydroxylase activity; and/or
(iii) a heterologous nucleic acid sequence coding for a 4-methoxybenzoate O-demethylase, preferably comprising a sequence chosen from SEQ ID NOs: 28 and 29 and polypeptides comprising a sequence having at least 60, 70, 80, 85, 90 or 95% sequence identity with one of these sequences and exhibiting L-tyrosine hydrolase activity.

Preferably, said microorganism comprises a heterologous nucleic acid sequence coding for a 4-hydroxyphenylacetate 3-monooxygenase oxygenase and a heterologous nucleic acid sequence coding for a 4-hydroxyphenylacetate 3-monooxygenase reductase.

According to a particular embodiment, the microorganism according to the invention is used in a method for producing ferulic acid. Preferably, in this embodiment, the microorganism comprises (i) a heterologous nucleic acid sequence coding for a 4-coumaroyl-CoA ligase (4CL), a heterologous nucleic acid sequence coding for a coumaroyl-CoA 3-hydroxylase (CCoA3H), a heterologous nucleic acid sequence coding for an acyl-coenzyme A thioesterase and (ii) a heterologous nucleic acid sequence coding for a caffeoyl-CoA O-methyltransferase (CCoAMT) and/or a heterologous nucleic acid sequence coding for caffeic acid O-methyltransferase (COMT).

The microorganism may further comprise a heterologous nucleic acid sequence coding for a C3H, a heterologous nucleic acid sequence coding for a 4-methoxybenzoate 0-demethylase, a heterologous nucleic acid sequence coding for a TAL, a heterologous nucleic acid sequence coding for a phenylalanine ammonia lyase (PAL), a heterologous nucleic acid sequence coding for a cinnamate 4-hydroxylase (C4H), a heterologous nucleic acid sequence coding for a 4-hydroxyphenylacetate 3-monooxygenase oxygenase, and/or a heterologous nucleic acid sequence coding for a 4-hydroxyphenylacetate 3-monooxygenase reductase.

Preferably, the microorganism further comprises a heterologous nucleic acid sequence coding for a TAL, a heterologous nucleic acid sequence coding for a phenylalanine ammonia lyase (PAL), a heterologous nucleic acid sequence coding for a cinnamate 4-hydroxylase (C4H), a heterologous nucleic acid sequence coding for a 4-hydroxyphenylacetate 3-monooxygenase oxygenase, and a heterologous nucleic acid sequence coding for a 4-hydroxyphenylacetate 3-monooxygenase reductase. The enzymes and combinations of enzymes are as described above.

Preferably, the cultivation of the microorganism is carried out without the addition of intermediate compound to the medium, i.e. without the addition of tyrosine, phenylalanine, cinnamic acid, p-coumaric acid, caffeic acid, caffeoyl-CoA and/or feruloyl-CoA.

Preferably, in this embodiment, the microorganism comprises
a heterologous nucleic acid sequence coding for a 4-coumaroyl-CoA ligase (4CL), a heterologous nucleic acid sequence coding for a coumaroyl-CoA 3-hydroxylase (CCoA3H) and a heterologous nucleic acid sequence coding for an acyl-coenzyme A thioesterase;
a heterologous nucleic acid sequence coding for a caffeoyl-CoA O-methyltransferase (CCoAMT) and/or a heterologous nucleic acid sequence coding for caffeic acid O-methyltransferase (COMT); and
a heterologous nucleic acid sequence coding for a tyrosine ammonia lyase (TAL), a heterologous nucleic acid sequence coding for a phenylalanine ammonia lyase (PAL), a heterologous nucleic acid sequence coding for a cinnamate 4-hydroxylase (C4H), each of these enzymes being as defined above, and combinations thereof.

According to a particular embodiment, the microorganism according to the invention used in a method for producing ferulic acid comprises:
(i)—a heterologous nucleic acid sequence coding for a 4CL comprising a sequence chosen from sequences SEQ ID NOs: 5 to 9 and polypeptides comprising a sequence having at least 60, 70, 80, 85, 90 or 95% sequence identity with any of these sequences and exhibiting 4CL activity; preferably a 4CL comprising a sequence chosen from sequences SEQ ID NOs: 5, 6, 8 and 9 and polypeptides comprising a sequence having at least 60, 70, 80, 85, 90 or 95% sequence identity with any of these sequences and exhibiting 4CL activity, and more particularly preferably a 4CL comprising a sequence chosen from sequences SEQ ID NOs: 6 and 8 and polypeptides comprising a sequence having at least 60, 70, 80, 85, 90 or 95% sequence identity with any of these sequences and exhibiting 4CL activity; and
a heterologous nucleic acid sequence coding for a CCoA3H comprising a sequence chosen from sequences SEQ ID NOs: 42 to 67 and polypeptides comprising a sequence having at least 60, 70, 80, 85, 90 or 95% sequence identity with any of these sequences and exhibiting CCoA3H activity; preferably a CCoA3H comprising a sequence chosen from sequences SEQ ID NO: 42, 45 and 49 and polypeptides comprising a sequence having at least 60, 70, 80, 85, 90 or 95% sequence identity with any of these sequences and exhibiting CCoA3H activity, and more particularly preferably a CCoA3H comprising a sequence chosen from sequence SEQ ID NO: 49 and polypeptides comprising a sequence having at least 60, 70, 80, 85, 90 or 95% sequence identity with said sequence and exhibiting CCoA3H activity; and
a heterologous nucleic acid sequence coding for an acyl-coenzyme A thioesterase comprising a sequence chosen from sequences SEQ ID NOs: 1, 2 and 39, and polypeptides comprising a sequence having at least 60, 70, 80, 85, 90 or 95% sequence identity with any of these sequences and exhibiting acyl-coenzyme A thioesterase activity, preferably an acyl-coenzyme A thioesterase comprising a sequence chosen from sequences SEQ ID NOs: 2 and 39 and polypeptides comprising a sequence having at least 60, 70, 80, 85, 90 or 95% sequence identity with any of these sequences and exhibiting acyl-coenzyme A thioesterase activity; and
(ii)—a heterologous nucleic acid sequence coding for a CCoAMT comprising a sequence chosen from sequences SEQ ID NOs: 10 to 16, 40 and 41 and polypeptides comprising a sequence having at least 60, 70, 80, 85, 90 or 95% sequence identity with any of these sequences and exhibiting CCoAMT activity, preferably a CCoAMT comprising a sequence chosen from sequence SEQ ID NO: 40 and polypeptides comprising a sequence having at least 60, 70, 80, 85, 90 or 95% sequence identity with said sequence and exhibiting CCoAMT activity, and/or
a heterologous nucleic acid sequence coding for a COMT comprising a sequence chosen from sequences SEQ ID NOs: 71 to 92, preferably SEQ ID NOs: 71 to 81, and polypeptides comprising a sequence having at least 60, 70, 80, 85, 90 or 95% sequence identity with any of these sequences, preferably with any of sequences SEQ ID NOs: 71 to 81, and exhibiting COMT activity, preferably a COMT comprising a sequence chosen from sequences SEQ ID NOs: 72 to 74, 76 and 79 to 92, preferably the sequences SEQ ID NOs: 72, 74, 76, 79, 80 and 82, and polypeptides comprising a sequence having at least 60, 70, 80, 85, 90 or 95% sequence identity with any of these sequences, preferably with any of sequences SEQ ID NOs: 72, 74, 76, 79, 80 and 82, and exhibiting COMT activity, and more particularly preferably a COMT comprising a sequence chosen from sequence SEQ ID NO: 72 and polypeptides comprising a sequence having at least 60, 70, 80, 85, 90 or 95% sequence identity with said sequence and exhibiting COMT activity.

It may further comprise
a heterologous nucleic acid sequence coding for a TAL comprising a sequence chosen from sequences SEQ ID NOs: 19, 30 and 68 and polypeptides comprising a sequence having at least 60, 70, 80, 85, 90 or 95% sequence identity with one of these sequences and exhibiting TAL activity, preferably a TAL comprising a sequence chosen from sequences SEQ ID NO: 19 and 68, preferably SEQ ID NO: 19, and polypeptides comprising a sequence having at least 60, 70, 80, 85, 90 or 95% sequence identity with any of these sequences, preferably with SEQ ID NO: 19, and exhibiting TAL activity, and
a heterologous nucleic acid sequence coding for a phenylalanine ammonia lyase (PAL) comprising a sequence chosen from SEQ ID NOs: 20, 69, 31 and 32 and polypeptides comprising a sequence having at least 60, 70, 80, 85, 90 or 95% sequence identity with any of these sequences and exhibiting PAL activity, and more particularly preferably, comprising a sequence chosen from sequences SEQ ID NOs: 20, 32 and 69, preferably SEQ ID NOs: 20 and polypeptides comprising a sequence having at least 60, 70, 80, 85, 90 or 95% sequence identity with one of these sequences, preferably with SEQ ID NO: 20, and exhibiting PAL activity; and
a heterologous nucleic acid sequence coding for a cinnamate 4-hydroxylase (C4H) comprising a sequence chosen from SEQ ID NOs: 21, 70, 33 and 34 and polypeptides comprising a sequence having at least 60, 70, 80, 85, 90 or 95% sequence identity with any of these sequences and exhibiting C4H activity, and more particularly preferably, comprising a sequence chosen from sequences SEQ ID NOs: 21 and 70, preferably SEQ ID NO: 21, and polypeptides comprising a sequence having at least 60, 70, 80, 85, 90 or 95% sequence identity with any of these sequences, preferably with SEQ ID NO: 21, and exhibiting C4H activity.

According to a preferred embodiment, the microorganism according to the invention used in a method for producing ferulic acid comprises:
(i)—a heterologous nucleic acid sequence coding for a 4CL comprising a sequence chosen from sequences SEQ ID NOs: 6 and 8 and polypeptides comprising a sequence having at least 60, 70, 80, 85, 90 or 95% sequence identity with any of these sequences and exhibiting 4CL activity; and
a heterologous nucleic acid sequence coding for a CCoA3H comprising a sequence chosen from sequences SEQ ID NOs: 42, 45 and 49 and polypeptides comprising a sequence having at least 60, 70, 80, 85, 90 or 95% sequence identity with any of these sequences and exhibiting CCoA3H activity, and preferably a CCoA3H comprising a sequence chosen from sequence SEQ ID NO: 49 and polypeptides comprising a sequence having at least 60, 70, 80, 85, 90 or 95% sequence identity with said sequence and exhibiting CCoA3H activity; and
a heterologous nucleic acid sequence coding for an acyl-coenzyme A thioesterase comprising a sequence chosen from sequences SEQ ID NOs: 2 and 39 and polypeptides comprising a sequence having at least 60, 70, 80, 85, 90 or 95% sequence identity with any of these sequences and exhibiting acyl-coenzyme A thioesterase activity; and (ii)—a heterologous nucleic acid sequence coding for a CCoAMT comprising a sequence chosen from SEQ ID NO: 40 and polypeptides comprising a sequence having at least 60, 70, 80, 85, 90 or 95% sequence identity with said sequence and exhibiting CCoAMT activity; and/or
a heterologous nucleic acid sequence coding for a COMT comprising a sequence chosen from sequences SEQ ID NOs: 72, 74, 76, 79, 80 and 82, and polypeptides comprising a sequence having at least 60, 70, 80, 85, 90 or 95% sequence identity with any of these sequences and exhibiting COMT activity, preferably comprising a sequence chosen from sequence SEQ ID NO: 72 and polypeptides comprising a sequence having at least 60, 70, 80, 85, 90 or 95% sequence identity with said sequence and exhibiting COMT activity.

It may further comprise
a heterologous nucleic acid sequence coding for a TAL comprising a sequence chosen from sequences SEQ ID NOs: 19 and 68, preferably SEQ ID NO: 19, and polypeptides comprising a sequence having at least 60, 70, 80, 85, 90 or 95% sequence identity with any of these sequences, preferably with SEQ ID NO: 19, and exhibiting TAL activity; and
a heterologous nucleic acid sequence coding for a phenylalanine ammonia lyase (PAL) comprising a sequence chosen from sequences SEQ ID NOs: 20, 32 and 69, preferably SEQ ID NOs: 20 and polypeptides comprising a sequence having at least 60, 70, 80, 85, 90 or 95% sequence identity with one of these sequences, preferably with SEQ ID NO: 20, and exhibiting PAL activity; and
a heterologous nucleic acid sequence coding for a cinnamate 4-hydroxylase (C4H) comprising a sequence chosen from sequences SEQ ID NOs: 21 and 70, preferably SEQ ID NO: 21, and polypeptides comprising a sequence having at least 60, 70, 80, 85, 90 or 95% sequence identity with one of these sequences, preferably with SEQ ID NO: 21, and exhibiting C4H activity.

Optionally, said microorganism may comprise
(i)—a heterologous nucleic acid sequence coding for a 4-hydroxyphenylacetate 3-monooxygenase oxygenase, preferably comprising a sequence chosen from sequences SEQ ID NOs: 17 and 26 and polypeptides comprising a sequence having at least 60, 70, 80, 85, 90 or 95% sequence identity with any of these sequences and exhibiting 4-hydroxyphenylacetate 3-monooxygenase oxygenase activity, and more particularly preferably a sequence chosen from SEQ ID NOs: 17 and polypeptides comprising a sequence having at least 60, 70, 80, 85, 90 or 95% sequence identity with sequence SEQ ID NO: 17 and exhibiting a 4-hydroxyphenylacetate 3-monooxygenase oxygenase activity, and
a heterologous nucleic acid sequence coding for a 4-hydroxyphenylacetate 3-monooxygenase reductase, preferably comprising a sequence chosen from sequences SEQ ID NOs: 18 and 27 and polypeptides comprising a sequence having at least 60, 70, 80, 85, 90 or 95% sequence identity with any of these sequences and exhibiting 4-hydroxyphenylacetate 3-monooxygenase reductase activity, and more particularly preferably a sequence chosen from SEQ ID NOs: 18 and polypeptides comprising a sequence having at least 60, 70, 80, 85, 90 or 95% sequence identity with sequence SEQ ID NO: 18 and exhibiting a 4-hydroxyphenylacetate 3-monooxygenase reductase activity; and/or
(ii) a heterologous nucleic acid sequence coding for a C3H, preferably comprising a sequence chosen from sequence SEQ ID NO: 25 and polypeptides comprising a sequence having at least 60, 70, 80, 85, 90 or 95% sequence identity with sequence SEQ ID NO: 25 and exhibiting p-coumarate 3-hydroxylase activity; and/or
(iii) a heterologous nucleic acid sequence coding for a 4-methoxybenzoate O-demethylase, preferably comprising a sequence chosen from SEQ ID NOs: 28 and 29 and polypeptides comprising a sequence having at least 60, 70, 80, 85, 90 or 95% sequence identity with one of these sequences and exhibiting L-tyrosine hydrolase activity.

Preferably, said microorganism comprises a heterologous nucleic acid sequence coding for a 4-hydroxyphenylacetate 3-monooxygenase oxygenase and a heterologous nucleic acid sequence coding for a 4-hydroxyphenylacetate 3-monooxygenase reductase.

Optionally, in each of these embodiments, the microorganism further comprises a heterologous nucleic acid sequence coding for a cytochrome P450 reductase (CPR), as defined above.

According to another embodiment, the microorganism according to the invention is a recombinant microorganism comprising a heterologous nucleic acid sequence coding for a COMT as described above and is used in a method for producing ferulic acid. The various embodiments relating to the recombinant microorganism according to the invention comprising a heterologous nucleic acid sequence coding for a COMT as described above are also considered in this aspect.

Preferably, in this embodiment, the microorganism comprises
(i) a heterologous nucleic acid sequence coding for caffeic acid O-methyltransferase (COMT),
(ii) a) a heterologous nucleic acid sequence coding for a C3H and/or b) a heterologous nucleic acid sequence coding for a 4-hydroxyphenylacetate 3-monooxygenase oxygenase, and a heterologous nucleic acid sequence coding for a 4-hydroxyphenylacetate 3-monooxygenase reductase,
(iii) a heterologous nucleic acid sequence coding for a TAL, a heterologous nucleic acid sequence coding for a phenylalanine ammonia lyase (PAL) and a heterologous nucleic acid sequence coding for a cinnamate 4-hydroxylase (C4H), and
(iv) optionally, a heterologous nucleic acid sequence coding for a cytochrome P450 reductase (CPR).

The enzymes and combinations of enzymes are as described above.

Preferably, the cultivation of the microorganism is carried out without the addition of intermediate compound to the medium, i.e. without the addition of tyrosine, phenylalanine, cinnamic acid, p-coumaric acid and/or caffeic acid.

According to the invention, any cultivation method for the industrial-scale production of molecules of interest may be envisioned. Advantageously, the cultivation is performed in bioreactors, notably in batch, fed-batch, chemostat and/or continuous cultivation mode. Controlled vitamin feeding during the process may also be beneficial to productivity (Alfenore et aL., Appl Microbiol Biotechnol. 2002, 60:67-72).

The cultivation is generally performed in bioreactors, with possible solid and/or liquid preculturing steps in Erlenmeyer flasks, with a suitable culture medium.

In general, the conditions for cultivating the microorganisms according to the invention are readily adaptable by a person skilled in the art, as a function of the microorganism. For example, the cultivation temperature is notably, for yeasts, between 20° C. and 40° C., preferably between 28° C. and 40° C., and more particularly about 30° C. for S. cerevisiae. The microorganism according to the present invention may be cultivated for 1 to 30 days and preferably for 1 to 10 days.

All the references cited in this description are incorporated by reference into the present application. Other characteristics and advantages of the invention will become clearer on reading the following examples given by way of illustration and without limitation.

TABLE 1
DESCRIPTION OF SEQUENCES

| SEQ | |
|---|---|
| ID | |
| NO: | Description of sequences of amino acids |
| 1 | Arabidopsis thaliana (Thio1) acyl-coenzyme A thioesterase |
| 2 | Petunia hybrida (Thio3) acyl-coenzyme A thioesterase |
| 3 | Arabidopsis thaliana aldehyde dehydrogenase |
| 4 | Populus tomentosa cinnamoyl-CoA reductase |
| 5 | Arabidopsis thaliana (4CL1) 4-coumaroyl-CoA ligase |
| 6 | Citrus clementina (4CL5) 4-coumaroyl-CoA ligase |
| 7 | Arabidopsis thaliana (4CL7) 4-coumaroyl-CoA ligase |
| 8 | Populus tomentosa (4CLA) 4-coumaroyl-CoA ligase |
| 9 | Arabidopsis thaliana (4CLB) 4-coumaroyl-CoA ligase |
| 10 | Vitis vinifera (CCoAMT1) caffeoyl-CoA O-methyltransferase |
| 11 | Medicago sativa (CCoAMT2) caffeoyl-CoA |
| | O-methyltransferase |
| 12 | Eucalyptus globus (CCoAMT3) caffeoyl-CoA |
| | O-methyltransferase |
| 13 | Nicotiana tabacum (CCoAMT4) caffeoyl-CoA |
| | O-methyltransferase |
| 14 | Nicotiana tabacum (CCoAMT5) caffeoyl-CoA |
| | O-methyltransferase |
| 15 | Arabidopsis thaliana (CCoAMT6) caffeoyl-CoA |
| | O-methyltransferase |
| 16 | Populus trichocarpa (CCoAMT7) caffeoyl-CoA |
| | O-methyltransferase |
| 17 | Pseudomonas aeruginosa 4-hydroxyphenylacetate |
| | 3-monooxygenase oxygenase |
| 18 | Salmonella enterica 4-hydroxyphenylacetate 3-monooxygenase |
| | reductase |
| 19 | Rhodotorula glutinis Tyrosine ammonia lyase |
| 20 | Arabidopsis thaliana Phenylalanine ammonia lyase |
| 21 | Arabidopsis thaliana cinnamate 4-hydroxylase |
| 22 | Catharantus roseus cytochrome P450 reductase |
| 23 | Aro4^{FBR}, feedback-resistant |
| 24 | Aro7^{FBR}, feedback-resistant |
| 25 | Saccharothrix espanaensis coumarate 3-hydroxylase |
| 26 | Escherichia coli 4-hydroxyphenylacetate 3-monooxygenase |
| | oxygenase |
| 27 | Escherichia coli 4-hydroxyphenylacetate 3-monooxygenase |
| | reductase |
| 28 | Rhodopseudomonas palustris 4-methoxybenzoate O- |
| | demethylase |
| 29 | Beta vulgaris 4-methoxybenzoate O-demethylase |
| 30 | Flavobacterium johnsoniae tyrosine ammonia lyase |
| 31 | Citrus sinensis phenylalanine ammonia lyase |
| 32 | Citrus sinensis phenylalanine ammonia lyase |
| 33 | Citrus sinensis cinnamate 4-hydroxylase |
| 34 | Citrus sinensis cinnamate 4-hydroxylase |
| 35 | Saccharomyces cerevisiae cytochrome P450 reductase |
| 36 | Arabidopsis thaliana cytochrome P450 reductase |
| 37 | Arabidopsis thaliana cytochrome P450 reductase |
| 38 | Chimeric cytochrome P450 reductase |
| 39 | Oryza meyeriana var. granulata (Thio30) Acyl-coenzyme A |
| | thioesterase |
| 40 | Panicum virgatum (CCoAMT8) Caffeoyl-CoA O- |
| | methyltransferase |
| 41 | Rauvolfia serpentina (CCoAMT9) Caffeoyl-CoA O- |
| | methyltransferase |
| 42 | Vigna angularis (CcoA3H1) Coumaroyl-CoA 3-hydroxylase |
| 43 | Glycine max (CcoA3H2) Coumaroyl-CoA 3-hydroxylase |
| 44 | Jatropha curcas (CcoA3H3) Coumaroyl-CoA 3-hydroxylase |
| 45 | Acacia koa (CcoA3H5) Coumaroyl-CoA 3-hydroxylase |
| 46 | Populus tomentosa (CcoA3H7) Coumaroyl-CoA 3-hydroxylase |
| 47 | Populus alba x Populus grandidentata (CcoA3H11) |
| | Coumaroyl-CoA 3-hydroxylase |
| 48 | Triticum turgidum subsp. durum (CcoA3H12) Coumaroyl-CoA |
| | 3-hydroxylase |
| 49 | Salvia miltiorrhiza (CcoA3H14) Coumaroyl-CoA 3-hydroxylase |
| 50 | Cosmos sulphureus (CcoA3H15) Coumaroyl-CoA 3-hydroxylase |
| 51 | Trifolium pratense (CcoA3H16) Coumaroyl-CoA 3-hydroxylase |
| 52 | Lonicera japonica (CcoA3H18) Coumaroyl-CoA 3-hydroxylase |
| 53 | Nyssa sinensis (CcoA3H20) Coumaroyl-CoA 3-hydroxylase |
| 54 | Pyrus ussuriensis x Pyrus communis (CcoA3H21) |
| | Coumaroyl-CoA 3-hydroxylase |
| 55 | Eucalyptus grandis (CcoA3H24) Coumaroyl-CoA 3-hydroxylase |
| 56 | Gossypium raimondii (CcoA3H25) Coumaroyl-CoA 3-hydroxylase |
| 57 | Zostera marina (CcoA3H26) Coumaroyl-CoA 3-hydroxylase |
| 58 | Aquilegia coerulea (CcoA3H27) Coumaroyl-CoA 3-hydroxylase |
| 59 | Actinidia chinensis var. chinensis (CcoA3H28) Coumaroyl-CoA |
| | 3-hydroxylase |
| 60 | Medicago truncatula (CcoA3H30) Coumaroyl-CoA 3-hydroxylase |
| 61 | Malus baccata (CcoA3H31) Coumaroyl-CoA 3-hydroxylase |
| 62 | Ricinus communis (CcoA3H32) Coumaroyl-CoA 3-hydroxylase |
| 63 | Sorghum bicolor (CcoA3H33) Coumaroyl-CoA 3-hydroxylase |
| 64 | Populus euphratica (CcoA3H36) Coumaroyl-CoA 3-hydroxylase |
| 65 | Nicotiana tabacum (CcoA3H37) Coumaroyl-CoA 3-hydroxylase |
| 66 | Raphanus sativus (CcoA3H38) Coumaroyl-CoA 3-hydroxylase |
| 67 | Ipomoea nil (CcoA3H39) Coumaroyl-CoA 3-hydroxylase |
| 68 | Citrus sinensis Tyrosine ammonia lyase |
| 69 | Arabidopsis thaliana phenylalanine ammonia lyase |
| 70 | Panicum virgatum cinnamate 4-hydroxylase |
| 71 | Panicum virgatum caffeic acid-O-methyltransferase |
| 72 | Arabidopsis thaliana caffeic acid-O-methyltransferase |
| 73 | Catharanthus roseus caffeic acid-O-methyltransferase |
| 74 | Triticum aestivum caffeic acid-O-methyltransferase |
| 75 | Triticum aestivum caffeic acid-O-methyltransferase |
| 76 | Panicum virgatum caffeic acid-O-methyltransferase |
| 77 | Nicotiana tabacum caffeic acid-O-methyltransferase |
| 78 | Picea abies caffeic acid-O-methyltransferase |
| 79 | Zea mays caffeic acid-O-methyltransferase |
| 80 | Stylosanthes humilis caffeic acid-O-methyltransferase |
| 81 | Saccharum officinarum caffeic acid-O-methyltransferase |
| 82 | Zea mays caffeic acid-O-methyltransferase |
| 83 | Cucumis sativus caffeic acid-O-methyltransferase |
| 84 | Tarenaya hassleriana caffeic acid-O-methyltransferase |
| 85 | Ziziphus jujuba var. spinosa caffeic acid-O-methyltransferase |
| 86 | Cucurbita maxima caffeic acid-O-methyltransferase |
| 87 | Ipomoea nil caffeic acid-O-methyltransferase |
| 88 | Thalictrum tuberosum caffeic acid-O-methyltransferase |
| 89 | Punica granatum caffeic acid-O-methyltransferase |
| 90 | Brassica cretica caffeic acid-O-methyltransferase |
| 91 | Lycium chinense caffeic acid-O-methyltransferase |
| 92 | Acer yangbiense caffeic acid-O-methyltransferase |

### EXAMPLES

### Example 1

### Materials and methods

### Strains

The yeast strains used in the examples were obtained from Saccharomyces cerevisiae S288C (Mortimer R K and Johnston J R (1986) Genealogy of principal strains of the yeast genetic stock center. Genetics 113(1):35-43). This yeast has auxotrophy for uracil, tryptophan and leucine.

The constructions were carried out in the Escherichia cols MH1 strain before their transfer to yeast.

In all the strains constructed for this study, the AR010 (YDR380W) and FDC1 (YDR539W) genes were inactivated, i.e. by integration, in place of the open reading frame, of a linear DNA comprising a selection marker bounded by the upstream and downstream regions of the gene.

TABLE 2
List of strains constructed

| Name of | |
|---|---|
| the strain | Proteins encoded by the heterologous genes inserted |
| 221 | 4CL1 (Arabidopsis thaliana, SEQ ID NO: 5) |
| | CcoAMT6 (Arabidopsis thaliana, SEQ ID NO: 15) |
| 239 | 4CL1 (Arabidopsis thaliana, SEQ ID NO: 5) |
| | CcoAMT6 (Arabidopsis thaliana, SEQ ID NO: 15) |
| | Thio1 (Arabidopsis thaliana, SEQ ID NO: 1) |
| 345 | 4CL1 (Arabidopsis thaliana, SEQ ID NO: 5) |
| | CcoAMT6 (Arabidopsis thaliana, SEQ ID NO: 15) |
| | Thio3 (Petunia hybrida, SEQ ID NO: 2) |
| 214 | 4CL1 (Arabidopsis thaliana, SEQ ID NO: 5) |
| | CcoAMT6 (Arabidopsis thaliana, SEQ ID NO: 15) |
| | CCR1 (Populus tomentosa, SEQ ID NO: 4) |
| | ALDH (Arabidopsis thaliana, SEQ ID NO: 3) |
| 617 | 4CL5 (Citrus clementina, SEQ ID NO: 6) |
| | CCoAMT1 (Vitis vinifera, SEQ ID NO: 10) |
| 367 | 4CL5 (Citrus clementina, SEQ ID NO: 6) |
| | CCoAMT1 (Vitis vinifera, SEQ ID NO: 10) |
| | Thio3 (Petunia hybrida, SEQ ID NO: 2) |
| 616 | 4CL5 (Citrus clementina, SEQ ID NO: 6) |
| | CCoAMT1 (Vitis vinifera, SEQ ID NO: 10) |
| | CCR1 (Populus tomentosa, SEQ ID NO: 4) |
| | ALDH (Arabidopsis thaliana, SEQ ID NO: 3) |
| 334 | Thio3 (Petunia hybrida, SEQ ID NO: 2) |
| | 4CL1 (Arabidopsis thaliana, SEQ ID NO: 5) |
| 339 | Thio3 (Petunia hybrida, SEQ ID NO: 2) |
| | 4CL1 (Arabidopsis thaliana, SEQ ID NO: 5) |
| | CCoAMT1 (Vitis vinifera, SEQ ID NO: 10) |
| 340 | Thio3 (Petunia hybrida, SEQ ID NO: 2) |
| | 4CL1 (Arabidopsis thaliana, SEQ ID NO: 5) |
| | CcoAMT2 (Medicago sativa, SEQ ID NO: 11) |
| 341 | Thio3 (Petunia hybrida, SEQ ID NO: 2) |
| | 4CL1 (Arabidopsis thaliana, SEQ ID NO: 5) |
| | CcoAMT3 (Eucalyptus globus, SEQ ID NO: 12) |
| 342 | Thio3 (Petunia hybrida, SEQ ID NO: 2) |
| | 4CL1 (Arabidopsis thaliana, SEQ ID NO: 5) |
| | CcoAMT4 (Nicotiana tabacum, SEQ ID NO: 13) |
| 343 | Thio3 (Petunia hybrida, SEQ ID NO: 2) |
| | 4CL1 (Arabidopsis thaliana, SEQ ID NO: 5) |
| | CcoAMT5 (Nicotiana tabacum, SEQ ID NO: 14) |
| 347 | Thio3 (Petunia hybrida, SEQ ID NO: 2) |
| | 4CL1 (Arabidopsis thaliana, SEQ ID NO: 5) |
| | CcoAMT7 (Populus trichocarpa, SEQ ID NO: 16) |
| 335 | Thio3 (Petunia hybrida, SEQ ID NO: 2) |
| | 4CL5 (Citrus clementina, SEQ ID NO: 6) |
| 368 | Thio3 (Petunia hybrida, SEQ ID NO: 2) |
| | 4CL5 (Citrus clementina, SEQ ID NO: 6) |
| | CcoAMT2 (Medicago sativa, SEQ ID NO: 11) |
| 369 | Thio3 (Petunia hybrida, SEQ ID NO: 2) |
| | 4CL5 (Citrus clementina, SEQ ID NO: 6) |
| | CcoAMT3 (Eucalyptus globus, SEQ ID NO: 12) |
| 370 | Thio3 (Petunia hybrida, SEQ ID NO: 2) |
| | 4CL5 (Citrus clementina, SEQ ID NO: 6) |
| | CcoAMT4 (Nicotiana tabacum, SEQ ID NO: 13) |
| 371 | Thio3 (Petunia hybrida, SEQ ID NO: 2) |
| | 4CL5 (Citrus clementina, SEQ ID NO: 6) |
| | CcoAMT5 (Nicotiana tabacum, SEQ ID NO: 14) |
| 373 | Thio3 (Petunia hybrida, SEQ ID NO: 2) |
| | 4CL5 (Citrus clementina, SEQ ID NO: 6) |
| | CcoAMT6 (Arabidopsis thaliana, SEQ ID NO: 15) |
| 375 | Thio3 (Petunia hybrida, SEQ ID NO: 2) |
| | 4CL5 (Citrus clementina, SEQ ID NO: 6) |
| | CcoAMT7 (Populus trichocarpa, SEQ ID NO: 16) |
| 336 | Thio3 (Petunia hybrida, SEQ ID NO: 2) |
| | 4CL7 (Arabidopsis thaliana, SEQ ID NO: 7) |
| 395 | Thio3 (Petunia hybrida, SEQ ID NO: 2) |
| | 4CL7 (Arabidopsis thaliana, SEQ ID NO: 7) |
| | CCoAMT1 (Vitis vinifera, SEQ ID NO: 10) |
| 396 | Thio3 (Petunia hybrida, SEQ ID NO: 2) |
| | 4CL7 (Arabidopsis thaliana, SEQ ID NO: 7) |
| | CcoAMT2 (Medicago sativa, SEQ ID NO: 11) |
| 397 | Thio3 (Petunia hybrida, SEQ ID NO: 2) |
| | 4CL7 (Arabidopsis thaliana, SEQ ID NO: 7) |
| | CcoAMT3 (Eucalyptus globus, SEQ ID NO: 12) |
| 398 | Thio3 (Petunia hybrida, SEQ ID NO: 2) |
| | 4CL7 (Arabidopsis thaliana, SEQ ID NO: 7) |
| | CcoAMT4 (Nicotiana tabacum, SEQ ID NO: 13) |
| 399 | Thio3 (Petunia hybrida, SEQ ID NO: 2) |
| | 4CL7 (Arabidopsis thaliana, SEQ ID NO: 7) |
| | CcoAMT5 (Nicotiana tabacum, SEQ ID NO: 14) |
| 401 | Thio3 (Petunia hybrida, SEQ ID NO: 2) |
| | 4CL7 (Arabidopsis thaliana, SEQ ID NO: 7) |
| | CcoAMT6 (Arabidopsis thaliana, SEQ ID NO: 15) |
| 403 | Thio3 (Petunia hybrida, SEQ ID NO: 2) |
| | 4CL7 (Arabidopsis thaliana, SEQ ID NO: 7) |
| | CcoAMT7 (Populus trichocarpa, SEQ ID NO: 16) |
| 337 | Thio3 (Petunia hybrida, SEQ ID NO: 2) |
| | 4CLA (Populus tomentosa, SEQ ID NO: 8) |
| 423 | Thio3 (Petunia hybrida, SEQ ID NO: 2) |
| | 4CLA (Populus tomentosa, SEQ ID NO: 8) |
| | CCoAMT1 (Vitis vinifera, SEQ ID NO: 10) |
| 424 | Thio3 (Petunia hybrida, SEQ ID NO: 2) |
| | 4CLA (Populus tomentosa, SEQ ID NO: 8) |
| | CcoAMT2 (Medicago sativa, SEQ ID NO: 11) |
| 425 | Thio3 (Petunia hybrida, SEQ ID NO: 2) |
| | 4CLA (Populus tomentosa, SEQ ID NO: 8) |
| | CcoAMT3 (Eucalyptus globus, SEQ ID NO: 12) |
| 426 | Thio3 (Petunia hybrida, SEQ ID NO: 2) |
| | 4CLA (Populus tomentosa, SEQ ID NO: 8) |
| | CcoAMT4 (Nicotiana tabacum, SEQ ID NO: 13) |
| 427 | Thio3 (Petunia hybrida, SEQ ID NO: 2) |
| | 4CLA (Populus tomentosa, SEQ ID NO: 8) |
| | CcoAMT5 (Nicotiana tabacum, SEQ ID NO: 14) |
| 429 | Thio3 (Petunia hybrida, SEQ ID NO: 2) |
| | 4CLA (Populus tomentosa, SEQ ID NO: 8) |
| | CcoAMT6 (Arabidopsis thaliana, SEQ ID NO: 15) |
| 431 | Thio3 (Petunia hybrida, SEQ ID NO: 2) |
| | 4CLA (Populus tomentosa, SEQ ID NO: 8) |
| | CcoAMT7 (Populus trichocarpa, SEQ ID NO: 16) |
| 338 | Thio3 (Petunia hybrida, SEQ ID NO: 2) |
| | 4CLB (Arabidopsis thaliana, SEQ ID NO: 9) |
| 451 | Thio3 (Petunia hybrida, SEQ ID NO: 2) |
| | 4CLB (Arabidopsis thaliana, SEQ ID NO: 9) |
| | CCoAMT1 (Vitis vinifera, SEQ ID NO: 10) |
| 452 | Thio3 (Petunia hybrida, SEQ ID NO: 2) |
| | 4CLB (Arabidopsis thaliana, SEQ ID NO: 9) |
| | CcoAMT2 (Medicago sativa, SEQ ID NO: 11) |
| 453 | Thio3 (Petunia hybrida, SEQ ID NO: 2) |
| | 4CLB (Arabidopsis thaliana, SEQ ID NO: 9) |
| | CcoAMT3 (Eucalyptus globus, SEQ ID NO: 12) |
| 454 | Thio3 (Petunia hybrida, SEQ ID NO: 2) |
| | 4CLB (Arabidopsis thaliana, SEQ ID NO: 9) |
| | CcoAMT4 (Nicotiana tabacum, SEQ ID NO: 13) |
| 455 | Thio3 (Petunia hybrida, SEQ ID NO: 2) |
| | 4CLB (Arabidopsis thaliana, SEQ ID NO: 9) |
| | CcoAMT5 (Nicotiana tabacum, SEQ ID NO: 14) |
| 457 | Thio3 (Petunia hybrida, SEQ ID NO: 2) |
| | 4CLB (Arabidopsis thaliana, SEQ ID NO: 9) |
| | CcoAMT6 (Arabidopsis thaliana, SEQ ID NO: 15) |
| 459 | Thio3 (Petunia hybrida, SEQ ID NO: 2) |
| | 4CLB (Arabidopsis thaliana, SEQ ID NO: 9) |
| | CcoAMT7 (Populus trichocarpa, SEQ ID NO: 16) |
| 516 | HpaB (Pseudomonas aeruginosa, SEQ ID NO: 17) |
| | HpaC (Salmonella enterica, SEQ ID NO: 18) |
| | TAL (Rhodotorula glutinis, SEQ ID NO: 19) |
| | PAL (Arabidopsis thaliana, SEQ ID NO: 20) |
| | C4H (Arabidopsis thaliana, SEQ ID NO: 21) |
| | CPR (Catharantus roseus, SEQ ID NO: 22) |
| | Aro4FBR (SEQ ID NO: 23) |
| | Aro7FBR (SEQ ID NO: 24) |
| 592 | HpaB (Pseudomonas aeruginosa, SEQ ID NO: 17) |
| | HpaC (Salmonella enterica, SEQ ID NO: 18) |
| | TAL (Rhodotorula glutinis, SEQ ID NO: 19) |
| | PAL (Arabidopsis thaliana, SEQ ID NO: 20) |
| | C4H (Arabidopsis thaliana, SEQ ID NO: 21) |
| | CPR (Catharantus roseus, SEQ ID NO: 22) |
| | Aro4FBR (SEQ ID NO: 23) |
| | Aro7FBR (SEQ ID NO: 24) |
| | 4CL5 (Citrus clementina, SEQ ID NO: 6) |
| | CCoAMT1 (Vitis vinifera, SEQ ID NO: 10) |
| | Thio3 (Petunia hybrida, SEQ ID NO: 2) |
| 593 | HpaB (Pseudomonas aeruginosa, SEQ ID NO: 17) |
| | HpaC (Salmonella enterica, SEQ ID NO: 18) |
| | TAL (Rhodotorula glutinis, SEQ ID NO: 19) |
| | PAL (Arabidopsis thaliana, SEQ ID NO: 20) |
| | C4H (Arabidopsis thaliana, SEQ ID NO: 21) |
| | CPR (Catharantus roseus, SEQ ID NO: 22) |
| | Aro4FBR (SEQ ID NO: 23) |
| | Aro7FBR (SEQ ID NO: 24) |
| | Thio3 (Petunia hybrida, SEQ ID NO: 2) |
| | 4CL5 (Citrus clementina, SEQ ID NO: 6) |
| | CCoAMT6 (Arabidopsis thaliana, SEQ ID NO: 15) |
| 594 | HpaB (Pseudomonas aeruginosa, SEQ ID NO: 17) |
| | HpaC (Salmonella enterica, SEQ ID NO: 18) |
| | TAL (Rhodotorula glutinis, SEQ ID NO: 19) |
| | PAL (Arabidopsis thaliana, SEQ ID NO: 20) |
| | C4H (Arabidopsis thaliana, SEQ ID NO: 21) |
| | CPR (Catharantus roseus, SEQ ID NO: 22) |
| | Aro4FBR (SEQ ID NO: 23) |
| | Aro7FBR (SEQ ID NO: 24) |
| | Thio3 (Petunia hybrida, SEQ ID NO: 2) |
| | 4CL5 (Citrus clementina, SEQ ID NO: 6) |
| | CcoAMT7 (Populus trichocarpa, SEQ ID NO: 16) |
| 507 | HpaB (Pseudomonas aeruginosa, SEQ ID NO: 17) |
| | HpaC (Salmonella enterica, SEQ ID NO: 18) |
| 595 | HpaB (Pseudomonas aeruginosa, SEQ ID NO: 17) |
| | HpaC (Salmonella enterica, SEQ ID NO: 18) |
| | 4CL5 (Citrus clementina, SEQ ID NO: 6) |
| | CCoAMT1 (Vitis vinifera, SEQ ID NO: 10) |
| | Thio3 (Petunia hybrida, SEQ ID NO: 2) |
| 596 | HpaB (Pseudomonas aeruginosa, SEQ ID NO: 17) |
| | HpaC (Salmonella enterica, SEQ ID NO: 18) |
| | Thio3 (Petunia hybrida, SEQ ID NO: 2) |
| | 4CL5 (Citrus clementina, SEQ ID NO: 6) |
| | CCoAMT6 (Arabidopsis thaliana, SEQ ID NO: 15) |
| 597 | HpaB (Pseudomonas aeruginosa, SEQ ID NO: 17) |
| | HpaC (Salmonella enterica, SEQ ID NO: 18) |
| | Thio3 (Petunia hybrida, SEQ ID NO: 2) |
| | 4CL5 (Citrus clementina, SEQ ID NO: 6) |
| | CcoAMT7 (Populus trichocarpa, SEQ ID NO: 16) |
| 623 | Thio3 (Petunia hybrida, SEQ ID NO: 2) |
| | CCoAMT8 (Panicum virgatum, SEQ ID NO: 40) |
| | 4CL5 (Citrus clementina, SEQ ID NO: 6) |
| 806 | Thio30 (Oryza meyeriana var. granulata, SEQ ID NO: 39) |
| | 4CL5 (Citrus clementina, SEQ ID NO: 6) |
| | CCoAMT6 (Arabidopsis thaliana, SEQ ID NO: 15) |
| 912 | Thio3 (Petunia hybrida, SEQ ID NO: 2) |
| | CCoAMT9 (Rauvolfia serpentina, SEQ ID NO: 41) |
| | 4CL5 (Citrus clementina, SEQ ID NO: 6) |

### Gene Cloning

The genes whose codons have been optimized for expression in yeast were synthesized by Twist Biosciences, San Francisco, USA or DC Biosciences, Dundee, UK.

The genes ARO4 (GenBank accession number: NP_009808) and ARO7 (GenBank accession number: NP_015385) were amplified by PCR from genomic DNA of S. cerevisiae and then mutated to make their product resistant to feedback (FBR: feedback resistance) (Gold et al., Microb Cell Fact. 2015; 14:73).

The promoters and terminators (Wagner et al., Fungal Genet Biol. 2016; 89:126-136) were amplified by PCR from genomic DNA of S. cerevisiae.

The genes obtained by synthesis or by PCR comprise at the 5′ and 3′ ends a Bbs/(GAAGAC) or Bsal (GGTCTC) restriction site, compatible with the cloning system used. All the genes, promoters and terminators were cloned into the restriction sites of the vector pSBK.

The vector pSBK comprises the selection marker URA3, LEU2 or TRP1.

### Cultivation Conditions

The yeast strains were cultured for 72 h at 30° C., in a 24-well plate, with continuous stirring (200 RPM), in 1 ml of SD medium (Dutscher, Brumath, France) supplemented or not with CSM (Complete Supplement Mixture; Formedium, UK).

Glucose is added at 20 g/L and, when required, p-coumaric acid or caffeic acid was added to the medium at a concentration of 100 mg/L.

Each strain was inoculated at OD 0.2 from a 24 h preculture cultured under the same conditions.

### Standards

Standards for p-coumaric acid, caffeic acid and ferulic acid were obtained from Sigma-Aldrich.

### Analytical Method

Preparation of the samples: Samples of 100 μL are recovered for each experiment. 50 μL are transferred to a new plate, to which 50 μL of the internal standard solution are added. Each sample is subsequently homogenized by suction-discharge and then centrifuged for 5 min at 3000 rpm at ambient temperature. The final concentration of the internal standard (protocatechoic acid) is 0.5 mg/L.

Analysis by UHPLC-TQ: The samples were analyzed by a Vanquish-H UHPLC (Thermo) coupled with a UHPLC-TQ triple quadrupole (Thermo). The column is a Waters Acquity UPLC@ USST3 column (8 μm 2.1×100 mm) combined with an HSST3 1.8 μm 2.1×5 mm precolumn.

Mobile phase A is a solution of 0.1% formic acid in LC/MS grade water and mobile phase B is a solution of 0.1% formic acid in pure LC/MS grade acetonitrile. The column temperature is 50° C. and the temperature of the sample changer is 10° C.

TABLE 3
Chromatographic conditions for the
detection of molecules of interest:

| Time | Flow rate | Mobile | Mobile |
|---|---|---|---|
| (min) | (ml/min) | phase A (%) | phase B (%) |
| 0 | 0.5 | 90 | 10 |
| 3.5 | 0.5 | 72 | 28 |
| 5.5 | 0.5 | 72 | 28 |
| 5.7 | 0.5 | 90 | 10 |
| 6.8 | 0.5 | 90 | 10 |

The parameters of the electrospray source are:
positive mode spray voltage at 4000 V
curtain gas: at 50 (arbitrary unit)
auxiliary gas at 15 (arbitrary unit)
transfer tube temperature at 300° C.
vaporizer temperature at 300° C.

TABLE 4
Ions monitored and fragmentation
conditions for the molecules of interest:

| | Re- | | | | | |
|---|---|---|---|---|---|---|
| | tention | | Pre- | | Col- | RF |
| | time | | cursor | Daughter | lision | lens |
| Molecules | (min) | Polarity | ion | ion | energy | (V) |
| p-Coumaric | 2.21 | Negative | 162.9 | 119.054 | 14.55 | 87 |
| acid | | | | 93 | 31.15 | 87 |
| Transferulic | 2.67 | Negative | 192.95 | 149.06 | 11.33 | 93 |
| acid | | | | 178.018 | 12.46 | 93 |
| Caffeic acid | 2.69 | Negative | 178.9 | 135 | 15.31 | 91 |
| | | | | 107.071 | 21.34 | 9 |

### Results

Production of Ferulic Acid from Caffeic Acid

The gene coding for the thioesterase of Arabidopsis thaliana (Thio1, SEQ ID NO: 1) or Petunia hybrida (Thio3, SEQ ID NO: 2) was inserted into strain 221, which expresses the 4CL of Arabidopsis thaliana (4CL1, SEQ ID NO: 5) and the CCoAMT of Arabidopsis thaliana (CCoAMT6, SEQ ID NO: 15) and in which the Aro10 and Fdc genes have been inactivated (strain 221). The strains obtained are strains 345 and 239.

Genes coding for the CCR of Populus tomentosa (SEQ ID NO: 4) and for the ALDH of Arabidopsis thaliana (SEQ ID NO: 3) were also inserted into strain 221 to obtain strain 214.

Ferulic acid production is tested with each of these strains in the presence of 100 mg/L of caffeic acid. It is compared with that obtained with strain 221.

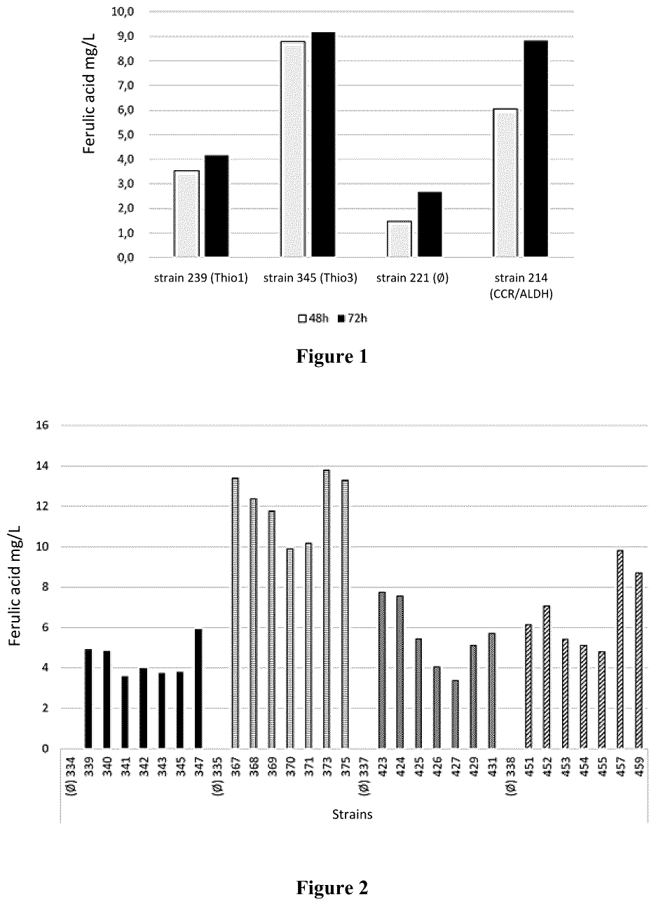

The results are presented in FIG. 1 and show that the plant thioesterases, CCR and ALDH expressed heterologously in yeast are active and capable of converting feruloyl-CoA into ferulic acid.

Similarly, the gene coding for the thioesterase of Oryza meyeriana var. granulata (Thio30, SEQ ID NO: 39) was inserted into a strain that expresses the 4CL of Citrus clementina (4CL5, SEQ ID NO: 6) and the CCoAMT of Arabidopsis thaliana (CCoAMT6, SEQ ID NO: 15) and in which the Aro10 and Fdc genes have been inactivated. The strain obtained is strain 806.

Ferulic acid production is tested in the presence of 100 mg/L of caffeic acid. It is compared with that obtained with a strain that expresses the 4CL of Citrus clementina (4CL5, SEQ ID NO: 6), the CCoAMT of Arabidopsis thaliana (CCoAMT6, SEQ ID NO: 15) and the thioesterase of Petunia hybrida (Thio3, SEQ ID NO: 2).

After 72 h of culture, strain 806 and the control strain produced 40 mg/L and 42 mg/L of ferulic acid, respectively. This result shows that the thioesterase from Oryza meyeriana var. granulata (Thio30, SEQ ID NO: 39) expressed heterologously in yeast is active and capable of converting feruloyl-CoA into ferulic acid.

Optimization of the Ferulic Acid Production Pathway from Caffeic Acid

Different yeast strains were constructed, all expressing the thioesterase of Petunia hybrida (Thio3, SEQ ID NO: 2) and in which the Aro10 and Fdc genes have been inactivated. Five different 4CLs (4CL1, 4CL5, 4CL7, 4CLA and 4CLB) were added to these strains. The 5 strains thus obtained are strains 334, 335, 336, 337 and 338.

These strains were then combined with seven different CCoAMTs (CCoAMT1, 2, 3, 4, 5, 6, 7, respectively SEQ ID NO: 10 to 16)). 35 strains were thus obtained: 339 to 343, 345, 347, 367 to 371, 373, 375, 395 to 399, 401, 403, 423 to 427, 429, 431, 451 to 455, 457 and 459.

Ferulic acid production is tested with each of these strains in the presence of 100 mg/L of caffeic acid. It is compared with that obtained with the control strains without CcoAMT, namely strains 334 to 338. The results are presented in FIG. 2.

The best combinations for the production of ferulic acid from caffeic acid are:
Thio3-4CL5-CCoAMT1 (strain 367)
Thio3-4CL5-CCoAMT6 (strain 373)
Thio3-4CL5-CCoAMT7 (strain 375)

The activity of the CCoAMTs alone (in the absence of 4CL and Thio3) was measured, to confirm that the synthesis of ferulic acid passes indeed through the CoA forms and is not obtained directly from caffeic acid.

In addition, strain 335 was combined with two different CCoAMTs (CCoAMT8 and 9, respectively SEQ ID NO: 40 and 41)). Strains 623 and 912 were thus obtained. Ferulic acid production was tested with each of these strains in the presence of 100 mg/L of caffeic acid. After 72 h of cultivation, strains 623 and 912 produced 42 mg/L and 41 mg/L of ferulic acid, respectively.

This result shows that the CCoAMTs of Panicum virgatum (CCoAMT8, SEQ ID NO: 40) and Rauvolfia serpentina (CCoAMT9, SEQ ID NO: 41) heterologously expressed in yeast are active and capable of converting caffeoyl-CoA into feruloyl-CoA, which is then converted into ferulic acid by the thioesterase.

Production of Ferulic Acid from p-Coumaric Acid

A yeast strain 507 was constructed in which the Aro10 and Fdc genes were inactivated and which expresses a gene coding for Pseudomonas aeruginosa HpaB (SEQ ID NO: 17) and a gene coding for Salmonella enterica HpaC (SEQ ID NO: 18). Strain 507 was then modified by insertion of the best-performing 4CL-CCoAMT-Thio combinations:
Strain 595: insertion of 4CL5 (Citrus sinensis, SEQ ID NO: 6), CCoAMT1 (Vitis vinifera, SEQ ID NO: 10) and Thio3 (Petunia hybrida, SEQ ID NO: 2);
Strain 596: insertion of 4CL5 (Citrus sinensis, SEQ ID NO: 6), CCoAMT7 (Populus trichocarpa, SEQ ID NO: 16) and Thio3 (Petunia hybrida, SEQ ID NO: 2); and
Strain 597: insertion of4CL5 (Citrus sinensis, SEQ ID NO: 6), CCoAMT6 (Arabidopsis thaliana, SEQ ID NO: 15) and Thio3 (Petunia hybrida, SEQ ID NO: 2).

These strains were then cultured in the presence of p-coumaric acid (100 mg/L).

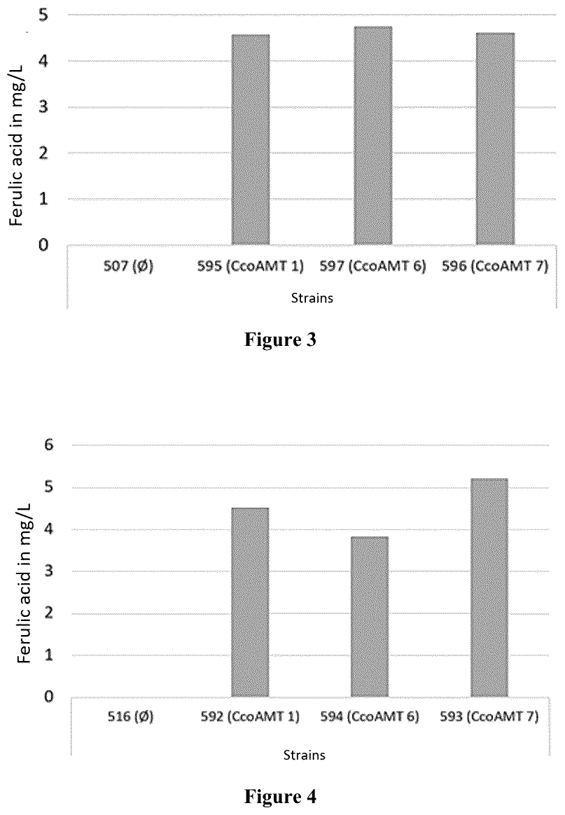

Ferulic acid production of these strains was measured after 24 h. The results are presented in FIG. 3.

### Insertion of a Complete Ferulic Acid Production Pathway in Yeast

A yeast strain 516 was constructed in which the Aro10 and Fdc genes were inactivated and which expresses a gene coding for a feedback-resistant Aro4 allele (AR04^{K229}L, SEQ ID NO: 23), a gene coding for a feedback-resistant Aro7 allele (ARO7G^{141S}, SEQ ID NO: 24), a gene coding for Pseudomonas aeruginosa HpaB (SEQ ID NO: 17), a gene coding for Salmonella enterica HpaC (SEQ ID NO: 18), a gene coding for Rhodotorula glutinis TAL (SEQ ID NO: 19), a gene coding for Arabidopsis thaliana PAL (SEQ ID NO: 20), a gene coding for Arabidopsis thaliana C4H (SEQ ID NO: 21) and a gene coding for Catharantus roseus CPR1 (SEQ ID NO: 22).
Strain 516 was then modified by insertion of the best-performing 4CL-CCoAMT-Thio combinations:
Strain 592: insertion of 4CL5 (Citrus sinensis, SEQ ID NO: 6), CCoAMT1 (Vitis vinifera, SEQ ID NO: 10) and Thio3 (Petunia hybrida, SEQ ID NO: 2);
Strain 593: insertion of 4CL5 (Citrus sinensis, SEQ ID NO: 6), CCoAMT7 (Populus trichocarpa, SEQ ID NO: 16) and Thio3 (Petunia hybrida, SEQ ID NO: 2); and
Strain 594: insertion of4CL5 (Citrus sinensis, SEQ ID NO: 6), CCoAMT6 (Arabidopsis thaliana, SEQ ID NO: 15) and Thio3 (Petunia hybrida, SEQ ID NO: 2).

These strains were then cultured in the presence of glucose (20 g/L).

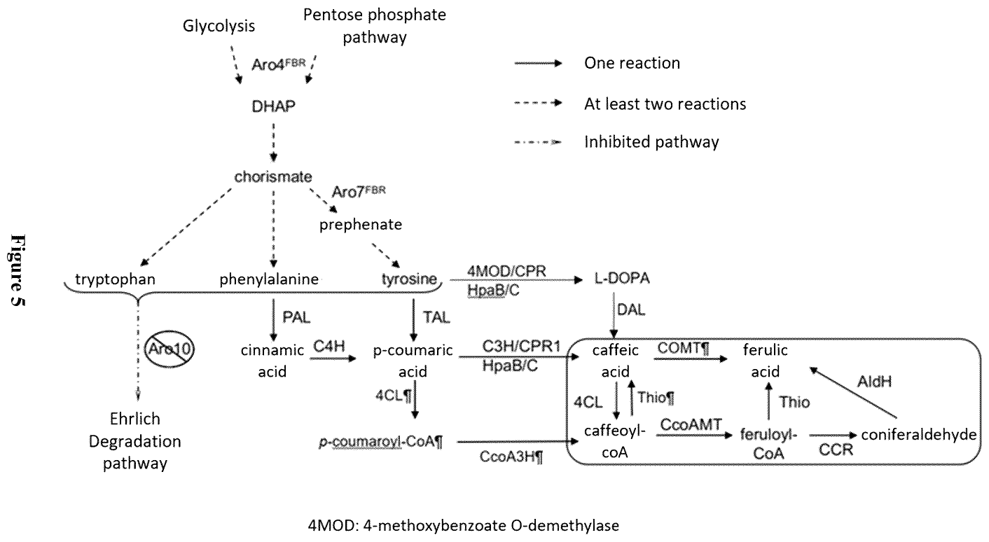

Ferulic acid production of these strains was measured after 24 h. The results are presented in FIG. 4.

### Example 2

### Materials and Methods

### Strains

The yeasts were obtained from the Saccharomyces cerevisiae strain FY1679-28A described in the article by Tettelin et al. (Methods in Molecular Genetics Volume 6, 1995, pages 81-107). This yeast has a quadruple auxotrophy for uracil, tryptophan, leucine and histidine. Cloning was carried out in the Escherichia coli MH1 strain.

### Standards

The standards were acquired from Sigma-Aldrich (p-coumaric acid, caffeic acid, ferulic acid).

### Gene Cloning

Genes optimized for expression in yeast were synthesized by Arurumolecular, Dundee, UK. The genes obtained by synthesis include at the 5′ and 3′ ends a Bbsl (GAAGAC) or Bsal (GGTCTC) restriction site.

All the genes, promoters and terminators were restriction-cloned into the vector pSBK for expression in yeast. The promoters and terminators (Wargner et al., Fungal Genet Biol. 2016 Apr; 89:126-136) were recovered by PCR from the genomic DNA of the yeast S. cerevisiae.

The pSBK vector comprises a yeast selection marker URA, or LEU or TRP or HIS, and a kanamycin resistance marker.

### Culture Conditions

The strains were cultured in 1 ml of medium (minimal nitrogen base for yeasts (Dutscher, Brumath, Fr) 6.7 g/L, glucose at 20 g/L, remainder CSM at 600 mg/L (Formedium, UK)) at 30° C. for 72 h with continuous stirring at 200 rpm. Each strain was inoculated at an optical density (OD) of 0.2 from a 24 h preculture cultured under the same conditions.

### Analytical Method: UHPLC-DAD-QExactive Method:

Sample preparation: 50 μL of acetonitrile and 50 μL of samples (dilution by 2) were injected into a 96-well plate. The plate was then stirred for 5 minutes at 35 rpm to homogenize the solvent and the sample. The plate was then centrifuged for 5 minutes at 3000 rpm.

UHPLC-DAD-QExactive analysis: The samples were analyzed by a Vanquish-H UHPLC (Thermo) coupled with a DAD detector (Thermo). The column is a Waters HSST3 C18 column (100×2.1 mm×1.8 μm) combined with an Acquity UPLC HSST3 VanGuard guard column.

Mobile phase A was a solution of 0.1% formic acid in LC/MS grade water and mobile phase B was a solution of 0.1% formic acid in pure LC/MS grade acetonitrile. The column temperature was 50° C. and the temperature of the sample changer was 10° C. (Tables 5 and 6)

TABLE 5
Chromatographic conditions for
the detection of molecules of interest

| Time | Flow rate | Mobile | Mobile |
|---|---|---|---|
| (min) | (ml/min) | phase A (%) | phase B (%) |
| 0 | 0.5 | 88 | 12 |
| 3.0 | 0.5 | 82 | 18 |
| 4.0 | 0.5 | 82 | 18 |
| 6.0 | 0.5 | 10 | 90 |
| 6.5 | 0.5 | 88 | 12 |
| 7.5 | 0.5 | 88 | 12 |

TABLE 6
Retention time and calibration range of molecules of interest

| Molecules | Retention time (min) | Calibration range (mg/L) |
|---|---|---|
| Caffeic acid | 1.80 | 3.9-500 |
| p-Coumaric acid | 2.89 | 3.9-500 |
| Transferulic acid | 3.51 | 7.03-900 |
| Isoferulic acid | 3.88 | 7.81-1000 |

### Results

### Hydroxylation of p-coumaroyl-CoA to caffeoyl-CoA by coumaroyl-CoA 3-hydroxylase enzymes

26 coumaroyl-CoA 3-hydroxylases (SEQ ID NO: 42 to 67) were tested in the strain named VAN305, corresponding to the S. cerevisiae strain FY1679-28A, expressing the genes coding for the enzymes of Rhodotorula glutinis TAL (SEQ ID NO: 19), Arabidopsis thaliana C4H (SEQ ID NO: 21), Arabidopsis thaliana PAL (SEQ ID NO: 69), Citrus clementina 4CL5 (SEQ ID NO: 6), Catharantus roseus CPR1 (SEQ ID NO: 22), Petunia hybrida Thio3 (SEQ ID NO: 2) and with deletion of the fdc1 gene.

The production of caffeic acid was tested with each of these coumaroyl-CoA 3-hydroxylases. Caffeic acid production was compared with the production of the control strain VAN305, which does not possess CcoA3H. The production was carried out from glucose (20 g/L) in 72 h.

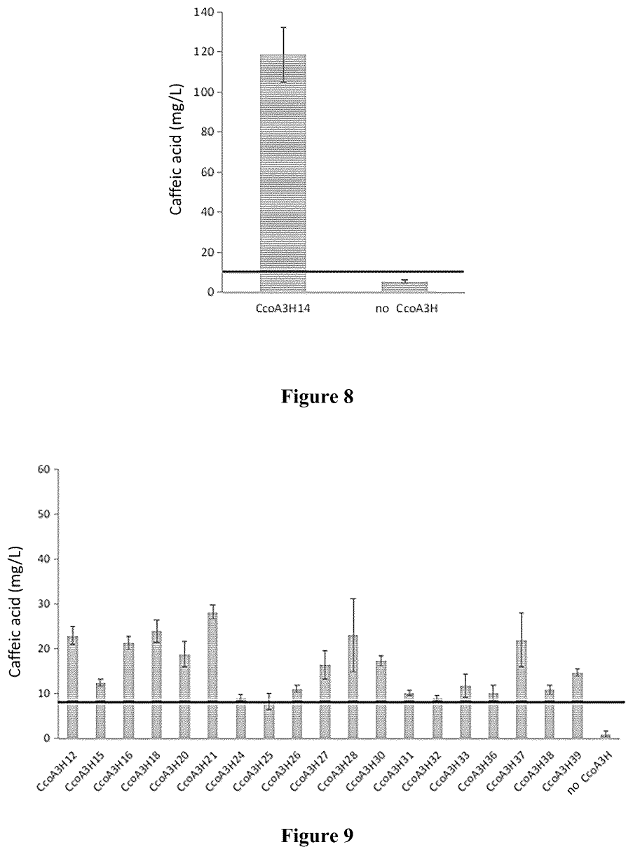

The caffeic acid production obtained for the strains expressing the different CcoA3H enzymes is presented in FIGS. 7 to 9.

To validate the activity of these enzymes on coumaroyl-CoA and not on p-coumaric acid, the enzymes were tested in the strain named VAN311, corresponding to the strain S. cerevisiae FY1679-28A, expressing the genes coding for the enzymes of Rhodotorula glutinis TAL (SEQ I|D NO: 19), Arabidopsis thaliana C4H (SEQ ID NO: 21), Arabidopsis thaliana PAL (SEQ ID NO: 69), Catharantus roseus CPR1 (SEQ ID NO: 22) and with deletion of the fdc1 gene. Caffeic acid production was then tested with each of the coumaroyl-CoA 3-hydroxylases. Caffeic acid production was compared with the production of the control strain VAN311, which does not possess a coumaroyl-CoA 3-hydroxylase enzyme.

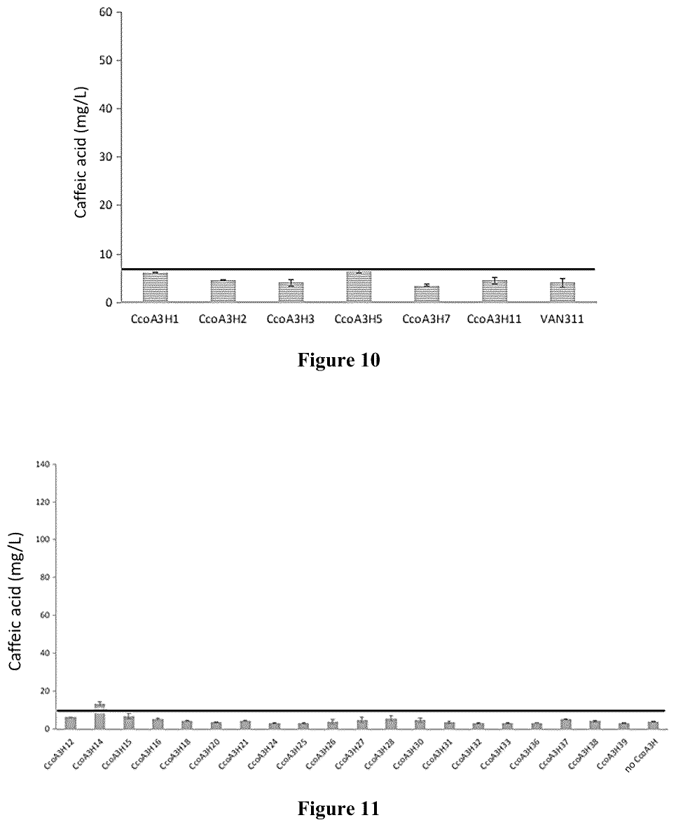

None of the coumaroyl-CoA 3-hydroxylases tested were able to add an —OH group at position 3 of p-coumaric acid to directly produce caffeic acid (FIGS. 10 and 11). This result thus confirms the action of CcoA3Hs on p-coumaroyl-CoA.

### Insertion of a Complete Ferulic Acid Production Pathway in Yeast Via the CoA Pathway

Ferulic acid production was tested from glucose (20 g/L). The test for ferulic acid production from glucose was performed in the S. cerevisiae strain FY1679-28A with deletion of the fdc1 gene (QAAfdc1). The following strains were constructed: VAN1622, VAN1624, YSP226 and YSP228. The enzymes expressed in each of these strains are presented in Table 7.

TABLE 7
Construction of strains
VAN1622, VAN1624, YSP226 and YSP228

| VAN1622 | VAN1624 | YSP226 | YSP228 |
|---|---|---|---|
| TAL + PAL + | TAL + PAL + | TAL + PAL + | TAL + PAL + |
| C4H + CPR1* | C4H + CPR1* | C4H + CPR1* | C4H + CPR1* |
| 4CL* | 4CL* | 4CL* | 4CL* |
| CcoA3H14* | CcoA3H14* | CcoA3H14* | CcoA3H14* |
| thio3* | thio30* | thio3* | thio30* |
| COMT | COMT | COMT | COMT |
| / | / | CcoAMT8* | CcoAMT8* |

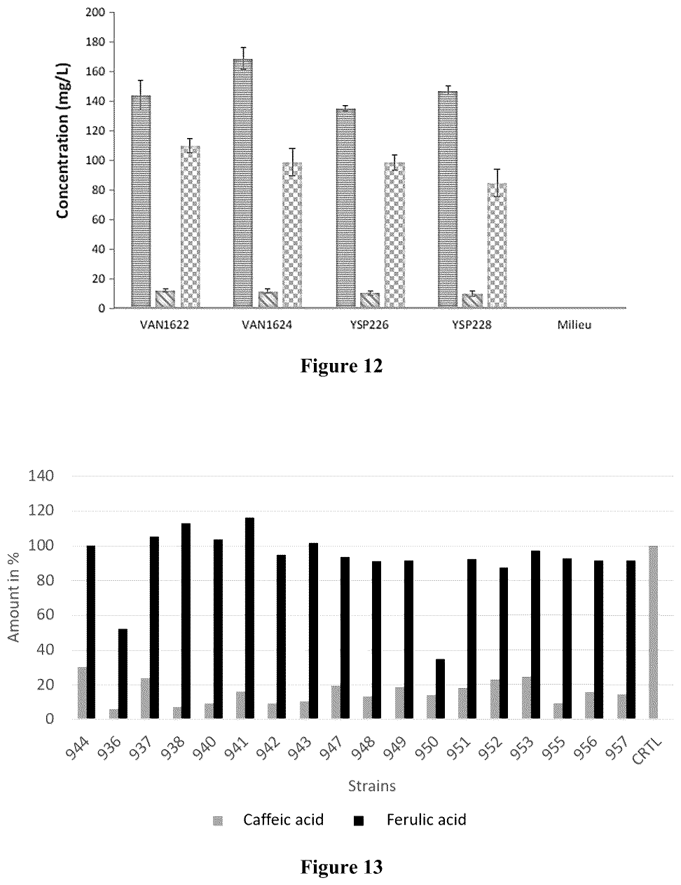

TAL + PAL + C4H + CPR1*: Rhodotorula glutinis TAL (SEQ ID NO: 19), Citrus sinensis TAL (SEQ ID NO: 68), Citrus sinensis PAL (SEQ ID NO: 32), Arabidopsis thaliana PAL (SEQ ID NO: 20), Arabidopsis thaliana C4H (SEQ ID NO: 21), Panicum virgatum C4H (SEQ ID NO: 70), Catharantus roseus CPR1 (SEQ ID NO: 22).
4CL*: Populus tomentosa 4CL (SEQ ID NO: 8); Salvia miltiorrhiza CcoA3H14* (SEQ ID NO: 49); Thio3: Petunia hybrida Thio3 (SEQ ID NO: 2), Oryza meyeriana var. granulata Thio30 (SEQ ID NO: 39); CcoAMT8: Panicum virgatum CcoAMT (SEQ ID NO: 40), Zea mays COMT (SEQ ID NO: 82).
Ferulic acid production of these strains is presented in FIG. 12.

### Example 3

### Materials and Methods

### Gene Cloning

Genes optimized for expression in yeast were synthesized by Twist Biosciences, San Francisco, USA or DC Biosciences, Dundee, UK. The genes obtained by synthesis comprise at the 5′ and 3′ ends a Bbsl (GAAGAC) or Bsal (GGTCTC) restriction site compatible with the cloning system used.

All the genes, promoters and terminators were restriction-cloned into the vector pSBK for expression in yeast. The promoters and terminators (Wargner et al., Fungal Genet Biol. 2016 Apr; 89:126-136) were recovered by PCR from the genomic DNA of the yeast S. cerevisiae.

The pSBK vector comprises a yeast selection marker HIS3, URA3, LEU2 or TRP1 and a kanamycin resistance marker.

### Strains

The yeasts were obtained from the Saccharomyces cerevisiae strain S288C (Mortimer RK and Johnston JR (1986) Genealogy of principal strains of the yeast genetic stock center. Genetics 113(1):35-43 PMID:3519363). This yeast has a quadruple auxotrophy for uracil, tryptophan, leucine and histidine.

In all the strains constructed, the ARla (YDR38QW) and FDC1 (YDR539W) genes were inactivated, i.e. by integration, in place of the open reading frame, of a linear DNA comprising a selection marker bounded by the upstream and downstream regions of the gene.

Cloning was carried out in the mscherichia coHi Mp(1 strain.

TABLE 8
List of strains constructed

| Name of | |
|---|---|
| the strain | Proteins encoded by the heterologous genes inserted |
| CRTL | Rhodotorula glutinis TAL (SEQ ID NO: 19) |
| (without | Arabidopsis thaliana PAL (SEQ ID NO: 20) |
| COMT) | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| 944 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Arabidopsis thaliana COMT (SEQ ID NO: 72) |
| 936 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Catharanthus roseus COMT (SEQ ID NO: 73) |
| 937 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Triticum aestivum COMT (SEQ ID NO: 74) |
| 938 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Panicum virgatum COMT (SEQ ID NO: 76) |
| 940 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Zea mays COMT (SEQ ID NO: 79) |
| 941 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Stylosanthes humilis COMT (SEQ ID NO: 80) |
| 942 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Saccharum officinarum COMT (SEQ ID NO: 81) |
| 943 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Zea mays COMT (SEQ ID NO: 82) |
| 947 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Cucumis sativus COMT (SEQ ID NO: 83) |
| 948 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Tarenaya hassleriana COMT (SEQ ID NO: 84) |
| 949 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Ziziphus jujuba var. spinosa COMT (SEQ ID NO: 85) |
| 950 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Cucurbita maxima COMT (SEQ ID NO: 86) |
| 951 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Ipomoea nil COMT (SEQ ID NO: 87) |
| 952 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Thalictrum tuberosum COMT (SEQ ID NO: 88) |
| 953 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Punica granatum COMT (SEQ ID NO: 89) |
| 955 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Brassica cretica COMT (SEQ ID NO: 90) |
| 956 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Lycium chinense COMT (SEQ ID NO: 91) |
| 957 | Rhodotorula glutinis TAL (SEQ ID NO: 19) |
| | Arabidopsis thaliana PAL (SEQ ID NO: 20) |
| | Arabidopsis thaliana C4H (SEQ ID NO: 21) |
| | Catharantus roseus CPR1 (SEQ ID NO: 22) |
| | Pseudomonas aeruginosa HpaB (SEQ ID NO: 17) |
| | Salmonella enterica HpaC (SEQ ID NO: 18) |
| | Acer yangbiense COMT (SEQ ID NO: 92) |

### Cultivation Conditions

The strains were cultured at 30° C. for 72 h with continuous stirring at 200 rpm in 1 ml of YNB medium (Dutscher, Brumath, Fr) supplemented with CSM (Complete Supplement Mixture; Formedium, UK). Glucose is added at 20 g/L.

### Standards

The standards were acquired from Sigma-Aldrich (p-coumaric acid, caffeic acid, ferulic acid).

### Analytical Method: UHPLC-TQ Method:

Sample preparation: Samples of 100 μL are recovered for each experiment. 50 μL are transferred to a new plate, to which 50 μL of the internal standard solution are added. Each sample is subsequently homogenized by suction-discharge and then centrifuged for 5 min at 3000 rpm at ambient temperature. The final concentration of the internal standard (protocatechic acid) is 0.5 mg/L.

UHPLC-TQ analysis: The samples were analyzed by a Vanquish-H UHPLC (Thermo) coupled with a UHPLC-TQ triple quadrupole (Thermo). The column is a Waters Acquity UPLC®USST3 column (8 μm 2.1×100 mm) combined with an HSST3 1.8 μm 2.1×5 mm precolumn.

Mobile phase A is a solution of 0.1% formic acid in LC/MS grade water and mobile phase B is a solution of 0.1% formic acid in pure LC/MS grade acetonitrile. The column temperature is 50° C. and the temperature of the sample changer is 10° C.

TABLE 9
Chromatographic conditions for
the detection of molecules of interest

| Time | Flow rate | Mobile | Mobile |
|---|---|---|---|
| (min) | (ml/min) | phase A (%) | phase B (%) |
| 0 | 0.5 | 90 | 10 |
| 3.5 | 0.5 | 72 | 28 |
| 5.5 | 0.5 | 72 | 28 |
| 5.7 | 0.5 | 90 | 10 |
| 6.8 | 0.5 | 90 | 10 |

### The Parameters of the Electrospray Source are:

positive mode spray voltage at 4000 V
curtain gas: at 50 AU
auxiliary gas at 15 AU
transfer tube temperature at 300° C.
vaporizer temperature at 300° C.

TABLE 10
Ions monitored and fragmentation conditions for the molecules of interest:

| | Retention | | Precursor | Daughter | Collision | |
|---|---|---|---|---|---|---|
| Molecules | time (min) | Polarity | ion | ion | energy | Lens RF (V) |
| p-Coumaric acid | 2.21 | Negative | 162.9 | 119.054 | 14.55 | 87 |
| | | | | 93 | 31.15 | 87 |
| Transferulic acid | 2.67 | Negative | 192.95 | 149.06 | 11.33 | 93 |
| | | | | 178.018 | 12.46 | 93 |
| Caffeic acid | 2.69 | Negative | 178.9 | 135 | 15.31 | 91 |
| | | | | 107.071 | 21.34 | 9 |
| | | | | 165 | 11.63 | 20 |

### Results

### Methylation of Caffeic Acid by COMT Enzymes

18 COMTs (SEQ ID NO: 71 to 74 and 79 to 92) were tested in the S. cerevisiae strain S288C, expressing the genes coding for the enzymes Rhodotorula glutinis TAL (SEQ ID NO: 19), Arabidopsis thaliana PAL (SEQ ID NO: 20), Arabidopsis thaliana C4H (SEQ ID NO: 21), Catharantus roseus CPR1 (SEQ ID NO: 22), Pseudomonas aeruginosa HpaB (SEQ ID NO: 17), Salmonella enterica HpaC (SEQ ID NO: 18) and with deletion of the fdc1 gene. Ferulic acid production was tested with each of these COMTs. Ferulic acid production was compared with the production of a control strain that includes the different enzymes except for a COMT (CRTL strain). The production was carried out from glucose (20 g/L) in 72 h.

Ferulic acid production obtained for strains expressing the different COMT enzymes is presented in FIG. 13.

The COMT-free strain ceases production at the stage where caffeic acid is formed and is not able to produce ferulic acid. The strains containing COMT are able to convert caffeic acid into ferulic acid.

The capacity to produce ferulic acid depends on the COMT enzyme used. Strains 937, 938, 940, 941 and 943, respectively expressing the COMTs of SEQ ID NO: 74, 76, 79, 80 and 82, show remarkable efficiency in producing ferulic acid from caffeic acid. In particular, these enzymes allow better conversion of caffeic acid into ferulic acid than the reference COMT enzyme of Arabidopsis thaliana.

1-36. (canceled)

37. A recombinant microorganism comprising a heterologous nucleic acid sequence coding a 4-coumaroyl-CoA ligase (4CL), a heterologous nucleic acid sequence coding a coumaroyl-CoA 3-hydroxylase (CCoA3H) and a heterologous nucleic acid sequence coding an acyl-coenzyme A thioesterase.

38. The microorganism according to claim 37, wherein said 4CL comprises a sequence chosen from SEQ ID NOs: 5 to 9 and polypeptides comprising a sequence having at least 60% sequence identity with one of these sequences and exhibiting 4CL activity.

39. The microorganism according to claim 37, wherein said CCoA3H comprises a sequence chosen from SEQ ID NOs: 42 to 67 and polypeptides comprising a sequence having at least 60% sequence identity with one of these sequences and exhibiting CCoA3H activity.

40. The microorganism according to claim 37, wherein said acyl-coenzyme A thioesterase comprises a sequence chosen from SEQ ID NOs: 1, 2 and 39 and polypeptides comprising a sequence having at least 60% sequence identity with one of these sequences and exhibiting acyl-coenzyme A thioesterase activity.

41. The microorganism according to claim 37, wherein the recombinant microorganism additionally comprises a heterologous nucleic acid sequence coding a caffeoyl-CoA O-methyltransferase (CCoAMT) comprising a sequence chosen from SEQ ID NOs: 10 to 16, 40 and 41, and polypeptides comprising a sequence having at least 60% sequence identity with one of these sequences and exhibiting CCoAMT activity.

42. The microorganism according to claim 41, wherein the recombinant microorganism additionally comprises a heterologous nucleic acid sequence coding a CCR and a heterologous nucleic acid sequence coding for an ALDH, said CCR comprising a sequence chosen from SEQ ID NO: 4 and polypeptides comprising a sequence having at least 60% sequence identity with SEQ ID NO: 4 and exhibiting CCR activity; and/or said ALDH comprises a sequence chosen from SEQ ID NO: 3 and polypeptides comprising a sequence having at least 60% sequence identity with SEQ ID NO: 3 and exhibiting ALDH activity.

43. The microorganism according to claim 37, wherein the recombinant microorganism additionally comprises a heterologous nucleic acid sequence coding for caffeic acid O-methyltransferase (COMT) comprising a sequence chosen from SEQ ID Nos: 72 to 92, and polypeptides comprising a sequence having at least 60% sequence identity with one of these sequences and exhibiting COMT activity.

44. The microorganism according to claim 37, wherein said microorganism is a bacterium, a yeast or a fungus.

45. The microorganism according to claim 37, wherein said recombinant microorganism additionally comprises
a heterologous nucleic acid sequence coding for a tyrosine ammonia lyase (TAL) comprising a sequence chosen from SEQ ID NOs: 19, 30 and 68 and polypeptides comprising a sequence having at least 60% sequence identity with SEQ ID NO: 19, 30 or 68 and exhibiting a tyrosine ammonia lyase activity; and/or
a heterologous nucleic acid sequence coding for a phenylalanine ammonia lyase (PAL) and a heterologous nucleic acid sequence coding for a cinnamate 4-hydroxylase (C4H), said phenylalanine ammonia lyase comprising a sequence chosen from SEQ ID NOs: 20, 69, 31 and 32 and polypeptides comprising a sequence having at least 60% sequence identity with SEQ ID NO: 20, 69, 31 or 32 and exhibiting a phenylalanine ammonia lyase activity and said cinnamate 4-hydroxylase comprising a sequence chosen from SEQ ID NOs: 21, 33, 34 and 70 and polypeptides comprising a sequence having at least 60% sequence identity with SEQ ID NO: 21, 33, 34 or 70 and exhibiting a cinnamate 4-hydroxylase activity.

46. The microorganism according to claim 37, wherein the recombinant microorganism additionally comprises a heterologous nucleic acid sequence coding for a phospho-2-dehydro-3-deoxyheptonate aldolase that is resistant to feedback by tyrosine and/or a heterologous nucleic acid sequence coding for a chorismate mutase that is resistant to feedback by tyrosine.

47. The microorganism according to claim 37, wherein, in said recombinant microorganism, a gene coding for a phenylpyruvate decarboxylase is inactivated and/or a gene coding for a ferulic acid decarboxylase is inactivated.

48. A method for producing a phenylpropanoid chosen from caffeic acid and ferulic acid, comprising culturing a recombinant microorganism as claimed in claim 37 and optionally harvesting and/or purifying said phenylpropanoid.

49. The method according to claim 48, wherein the phenylpropanoid is ferulic acid and the recombinant microorganism is a microorganism comprising (i) a heterologous nucleic acid sequence coding for a 4-coumaroyl-CoA ligase (4CL), a heterologous nucleic acid sequence coding for a coumaroyl-CoA 3-hydroxylase (CCoA3H), a heterologous nucleic acid sequence coding for an acyl-coenzyme A thioesterase and (ii) a heterologous nucleic acid sequence coding for a caffeoyl-CoA O-methyltransferase (CCoAMT) and/or a heterologous nucleic acid sequence coding for caffeic acid O-methyltransferase (COMT).

50. A recombinant microorganism comprising a heterologous nucleic acid sequence coding for a caffeic acid O-methyltransferase (COMT), said COMT comprising a sequence chosen from SEQ ID NOs: 73 to 92, and polypeptides comprising a sequence having at least 60%, sequence identity with one of these sequences and exhibiting COMT activity.

51. The microorganism according to claim 50, wherein the recombinant microorganism further comprises a heterologous nucleic acid sequence coding for a 4-hydroxyphenylacetate 3-monooxygenase oxygenase (HpaB) and a heterologous nucleic acid sequence coding for a 4-hydroxyphenylacetate 3-monooxygenase reductase (HpaC).

52. The microorganism according to claim 51, wherein:
said HpaB comprises a sequence chosen from SEQ ID NOs: 17 and 26 and the polypeptides comprising a sequence having at least 60% sequence identity with one of these sequences and exhibiting 4-hydroxyphenylacetate 3-monooxygenase oxygenase activity; and said HpaC comprises a sequence chosen from SEQ ID NOs: 18 and 27 and polypeptides comprising a sequence having at least 60% sequence identity with one of these sequences and exhibiting 4-hydroxyphenylacetate 3-monooxygenase reductase activity.

53. The microorganism according to claim 50, wherein the recombinant microorganism comprises a heterologous nucleic acid sequence coding for a CPR-dependent p-coumarate 3-hydroxylase (C3H) comprising a sequence chosen from SEQ ID NO: 25 and the polypeptides comprising a sequence having at least 60% sequence identity with SEQ ID NO: 25 and exhibiting a p-coumarate 3-hydroxylase activity.

54. The microorganism according to claim 50, wherein the recombinant microorganism further comprises a heterologous nucleic acid sequence coding for a cytochrome P450 reductase (CPR).

55. The microorganism according to claim 50, wherein said microorganism is a bacterium, a yeast or a fungus.

56. A method for producing ferulic acid, comprising culturing a recombinant microorganism according to claim 50 and optionally harvesting and/or purifying the ferulic acid produced.
